(19) United States
(12) Patent Application Publication
(10) Pub. No.: US 2025/0155441 A1
(43) Pub. Date: May 15, 2025

(54) HIGH AFFINITY ANTIBODIES SPECIFICALLY BINDING TO a-1,6-CORE-FUCOSYLATED ALPHA-FETOPROTEIN

(71) Applicant: Roche Diagnostics Operations, Inc., Indianapolis, IN (US)

(72) Inventors: Michael Gerg, Muenchen (DE); Lars Hillringhaus, Koenigsdorf (DE); Ute Jucknischke, Iffeldorf (DE); Anna Christine Muth, Germering (DE); Tobias Oelschlaegel, Geretsried-Gelting (DE); Boris Pinchuk, Weilheim in Oberbayern (DE); Michael Schraeml, Penzberg (DE); Joanna Siebenhaar, Penzberg (DE); Laurence Thirault, Penzberg (DE)

(21) Appl. No.: 18/951,180

(22) Filed: Nov. 18, 2024

Related U.S. Application Data
(63) Continuation of application No. PCT/EP2023/062762, filed on May 12, 2023.

(30) Foreign Application Priority Data
May 16, 2022 (EP) 22173621.8

(51) Int. Cl. G01N 33/574; C07K 16/18

(52) U.S. Cl. CPC G01N 33/57438; C07K 16/18; G01N 33/57488; C07K 2317/24; C07K 2317/92; G01N 2333/471; G01N 2440/38

(57) ABSTRACT

The present invention relates to monoclonal antibodies and antigen binding fragments that specifically bind to α-1,6-core-fucosylated alpha-fetoprotein (AFP), which is the core component of AFP-L3. Thus, the antibodies and antigen binding fragments provided herein may also be referred to as AFP-L3 antibodies. Also provided are polynucleotides encoding the antibodies or antigen binding fragments of the invention, host cells expressing the antibodies and antigen binding fragments of the invention, methods for producing the antibodies and antigen binding fragments of the invention, and uses of the antibodies and antigen binding fragments of the invention. Also provided herein is a pretreatment agent facilitating the binding of the antibodies and antigen binding fragments of the invention to α-1,6-core-fucosylated AFP. The present invention further relates to kits comprising the antibodies and antigen binding fragments of the invention and optionally the pretreatment agent of the invention.

### CROSS-REFERENCE TO RELATED APPLICATIONS

This application is a continuation of International PCT Application No. PCT/EP2023/062762 filed on May 12, 2023, which claims priority to European Patent Application No. 22173621.8 filed on May 16, 2022, the contents of each application are incorporated herein by reference in their entireties.

### SEQUENCE LISTING

This application incorporates by reference the material in the ST.26 XML file titled P37547-US_Sequence-Listing_ROCHE-215, which was created on Nov. 18, 2024 and is 33.4 KB.

### FIELD OF THE INVENTION

The present invention relates to monoclonal antibodies and antigen binding fragments that specifically bind to α-1,6-core-fucosylated alpha-fetoprotein (AFP), which is the core component of AFP-L3. Thus, the antibodies and antigen binding fragments provided herein may also be referred to as AFP-L3 antibodies. Also provided are polynucleotides encoding the antibodies or antigen binding fragments of the invention, host cells expressing the antibodies and antigen binding fragments of the invention, methods for producing the antibodies and antigen binding fragments of the invention, and uses of the antibodies and antigen binding fragments of the invention. Also provided herein is a pretreatment agent facilitating the binding of the antibodies and antigen binding fragments of the invention to α-1,6-core-fucosylated AFP. The present invention further relates to kits comprising the antibodies and antigen binding fragments of the invention and optionally the pretreatment agent of the invention.

### BACKGROUND OF THE INVENTION

Liver cancer is the seventh most common cancer and the second cause of death from cancer worldwide.

Hepatocellular carcinoma (HCC) is the major histologic type among primary liver cancers occurring worldwide, accounting for 70% to 85% of the total burden. It is known, that underlying liver diseases such as liver fibrosis and cirrhosis are the main risk factors for the development of HCC. HCC can be treated by resection, liver transplantation, or local ablation with radiofrequency for patients diagnosed at an early stage. Thus, in the case of HCC early stage detection and a minimal invasive screening method are of crucial importance.

The most common methods for diagnosis of HCC are ultrasound detection, imaging techniques such as computed axial tomography (CAT scan) or magnetic resonance imaging (MRI) and serological biomarkers. However, the ultra-sound detection has poor prognosis value, as it requires at least a 2 cm tumor mass and the imaging techniques have poor sensitivity of a per lesion basis and high costs. On the other hand, a lot of focus has been put to discover new blood biomarker that can be used in surveillance programs for early detection of HCC in high-risk patients in recent years (Yang J D. Detect or not to detect very early stage hepatocellular carcinoma? The western perspective. Clin Mol Hepatol. 2019; 25(4):335-43).

Several current medical guidelines recommend surveillance of high-risk patients using ultrasound, or other imaging modalities, every 6 months. However, limitations of the imaging approach include poor sensitivity for early stage tumors, operator-dependency, and lower quality in patients with obesity or non-alcoholic steatohepatitis.

α-Fetoprotein (AFP) is the currently best established biomarker for hepatocellular carcinoma (HCC). However, there is a need to further improve the sensitivity and specificity of AFP for HCC diagnosis, especially early stage HCC diagnosis.

AFP is a glycoprotein and various glycosylated forms of AFP have been described. In particular, AFP is N-glycosylated at asparagine 251. Lectins can be used in the analysis of glycoproteins. By using the selective binding capacity of a lectin to the sugar chain structure of a glycoprotein it is possible to separate and concentrate the marker glycoprotein fraction(s) having a specific sugar chain structure. In the case of AFP, the lectin derived from Lens culinaris agglutinin-A (LCA) has been widely used. Using LCA, AFP can be fractionated into the three variants L1, L2 and L3, wherein AFP-L3 has the highest affinity to LCA. The AFP-L3 fraction is composed of AFP, which is N-glycosylated at Asn-251 with an N-glycan comprising α-1,6-core-fucosylation (i.e., AFP having a fucose sugar bound to N-acetylglucosamine (GlcNAc), which is located at a reducing terminal of an N-type sugar chain via an α-1,6 bond). Accordingly, as AFP-L3 is composed of α-1,6-core-fucosylated AFP, the terms AFP-L3 and α-1,6-core-fucosylated AFP are used herein interchangeably. The Lens culinaris agglutinin (LCA)-reactive fraction of α-fetoprotein (AFP-L3) is specifically increased in patients with HCC (Khien V V et al., The International Journal of Biological Markers. 2001; 16(2):105-111).

Recent publications clearly point out that scores comprising the input variables gender, age, AFP and descarboxyprothrombin (DCP=PIVKA-II) (=GAAD-score) or gender, age, AFP, AFP-L3 and descarboxyprothrombin (DCP=PIVKA-II) (=GALAD-score) can and will improve the results of HCC-screening efforts and especially also the detection of early HCC (Zhou, J-M., Wang, T., Zhang, K-H. AFP-L3 for the diagnosis of early hepatocellular carcinoma, Medicine 2021; 100(43):p e27673).

Accordingly, there is a high need to reliably detect and quantify AFP-L3.

The μTASWako AFP-L3 assay (Fujifilm Wako is at present the most widely used method for detecting and quantifying AFP-L3. This assay is based on affinity capillary electrophoresis using LCA for separating AFP-L3. However, this method has certain shortcomings. First, N-glycan branching may lead to detection of wrong AFP-L3 levels. Second, the method is cumbersome and requires special instrumentation. Accordingly, there is a high desire to design antibody-based methods (Egashira Y et al., Scientific Reports, 2019, 9:12359). Third, lectins are less specific than antibodies and typically show only a low binding affinity.

JP S63-307900 discloses an antibody, which binds to LCA-binding AFP (AFP-LCA-R) but does not bind to LCA non-binding AFP (AFP-LCA-NR). However, an epitope for the antibody is unknown and it is unclear whether this antibody also recognizes proteins other than AFP with the same sugar moiety.

EP 3 252 073, US2018/0110889 and Egashira Y et al. (Scientific Reports, 2019, 9:12359) disclose monoclonal antibodies directed against core-fucosylated AFP (i.e. AFP-L3) which bind in a core-fucose dependent manner and also bind parts of the AFP peptide backbone. However, as well known in the art, and as stated in Egashira Y et al. (Scientific Reports, 2019, 9:12359), most anti-carbohydrate antibodies have low affinity for their antigens. While the affinity of the antibody FasMab is described as stronger than typical anti-carbohydrate antibodies, the K_{D} value is only 6.5×10⁻⁷ M. Typically, antibodies used in fully automated immunoassays such as Elecsys® assays (Roche) have significantly lower K_{D} values (e.g. in the low nanomolar range), i.e. higher affinity to AFP-L3.

Accordingly there is a need to provide novel antibodies specifically binding to α-1,6-core-fucosylated alpha-fetoprotein (i.e. AFP-L3) with improved kinetic properties, in particular with higher affinity (i.e. lower K_{D}). Further, there is a need for providing improved immunoassays for detecting AFP-L3.

### SUMMARY OF THE INVENTION

The above-mentioned needs are solved by the monoclonal antibodies and antigen binding fragments thereof as well as the uses thereof provided in the present invention.

In a first aspect of the present invention, provided are monoclonal antibodies or antigen binding fragment thereof specifically binding to α-1,6-core-fucosylated AFP or a partial sequence of AFP comprising said α-1,6-core-fucosylation.

The monoclonal antibodies and antigen binding fragments thereof provided herein discriminate α-1,6-core-fucosylated AFP (also referenced herein as core-fucosylated AFP and 1,6fucAFP) from related structures with high specificity. First, the monoclonal antibodies or antigen binding fragments thereof provided herein can discriminate α-1,6-core-fucosylated AFP from AFP lacking the α-1,6-core-fucose residue (i.e. aglycosylated AFP or AFP with N-glycans lacking an α-1,6-core-fucose residue). Second, the monoclonal antibodies or antigen binding fragments provided herein can discriminate α-1,6-core-fucosylated AFP from free N-glycans bearing an α-1,6-core-fucose residue. Third, the monoclonal antibodies or antigen binding fragments provided herein discriminate α-1,6-core-fucosylated AFP from aglycosylated AFP peptides comprising the N-glycosylation site N-251. Based on this binding behavior it can be concluded that the antibodies and the antigen binding fragments thereof bind α-1,6-core-fucosylated AFP both dependent on the presence of the α-1,6-core-fucosylation and an AFP peptide sequence, e.g. of SEQ ID NO:2. Accordingly, the antibodies and antigen binding fragments provided herein are specific for α-1,6-core-fucosylated AFP and do not significantly recognize (i) other proteins with similar α-1,6-core-fucosylated N-glycan structures and (ii) AFP or partial sequences thereof lacking the α-1,6-core-fucose residue.

With this specificity the antibodies and antigen-binding provided herein are valuable tools for use in immunoassays for detecting the level of α-1,6-core-fucosylated AFP (clinically equivalent to AFP-L3) in samples. Accordingly, the antibodies and antigen binding fragments and the immunoassays using the same can aid in the detection of HCC, in particular early HCC.

The antibodies and the antigen binding fragments provided herein show a surprisingly high affinity to α-1,6-core-fucosylated AFP as exemplified herein by analyzing the binding to an α-1,6-core-fucosylated AFP peptide. The K_{D} measured for such interaction is in the single digit nanomolar range, i.e. is order of magnitudes lower than the K_{D} that could have been expected based on the teaching in the prior art for such AFP glycopeptide antibody (Egashira Y et al., Scientific Reports, 2019, 9:12359). Egashira Y et al. have reported affinities of 650 nM as surprisingly low K_{D} (i.e. high binding affinity) for antibodies directed to α-1,6-core-fucosylated AFP, underlining that it is a unexpected finding that antibodies with an affinity as reported herein could be identified.

Interestingly, the two best monoclonal antibodies identified herein, namely the clones 19B12 and 3C5, show a very high sequence homology, especially also in the CDR residues. There is in total a difference of 3 amino acids in the CDR residues, one insertion and two amino acid substitution.

Accordingly, in embodiments, the monoclonal antibody or antigen binding fragment according to the first aspect of the present disclosure comprises
(i) a heavy chain variable domain (VH) comprising a CDR-H1 having the amino acid sequence of SEQ ID NO: 3 (CDR-H1 of 19B12 and 3C5) or a variant thereof modified by one amino acid substitution; a CDR-H2 having the amino acid sequence of SEQ ID NO: 4 or 5 (CDR-H2 of 19B12 and 3C5, respectively) or a variant of SEQ ID NO: 4 or 5 modified by at most two amino acid substitutions; and a CDR-H3 having the amino acid sequence of SEQ ID NO: 6 (CDR-H3 of 19B12 and 3C5) or a variant thereof modified by one amino acid substitution; and
(ii) a light chain variable domain (VL) comprising a CDR-L1 having the amino acid sequence of SEQ ID NO: 7 or 8 (CDR-L1 of 19B12 and 3C5, respectively) or a variant of SEQ ID NO: 7 or 8 modified by at most two amino acid substitutions; a CDR-L2 having the amino acid sequence of SEQ ID NO: 9 (CDR-L2 of 19B12 and 3C5) or a variant thereof modified by one amino acid substitution; and a CDR-L3 having the amino acid sequence of SEQ ID NO: 10 (CDR-L3 of 19B12 and 3C5) or a variant thereof modified by one amino acid substitution.

In a second aspect, the present invention provides a polynucleotide encoding
(i) the heavy chain or heavy chain variable domain of the monoclonal antibody or antigen binding fragments according to the first aspect and/or
(ii) the light chain or light chain variable domain of the monoclonal antibody or antigen binding fragment according to the first aspect.

In a third aspect, disclosed is a vector comprising the polynucleotide according to the second aspect of the invention.

According to a fourth aspect of the invention, herein provided is a host cell comprising the polynucleotide of the second aspect, or the vector of the third aspect.

In a fifth aspect, the present invention relates to a method of producing the monoclonal antibody or antigen binding fragment according to the first aspect, said method comprising culturing the host cell according to the fourth aspect and isolating said antibody or antigen binding fragment. Disclosed are also antibodies obtained or obtainable by such method.

According to a sixth aspect, provided is a composition comprising the monoclonal antibody or antigen binding fragment according to the first aspect, the polynucleotide according to the second aspect, the vector according to the third aspect, or the host cell according to the fourth aspect.

In embodiments of the sixth aspect, provided is a diagnostic composition comprising the antibody according to the first aspect of the invention.

Using the antibodies or antigen binding fragments of the invention, the present inventors have generated an immunoassay for the determining the level of α-1,6-core-fucosylated AFP. The levels α-1,6-core-fucosylated AFP determined with this assay correlate very well with the levels determined with the μTASWako AFP-L3 assay, which is at present the most widely used method for detecting and quantifying AFP-L3. Accordingly, the immunoassays using the antibodies of the invention are a highly reliable tool, e.g. for the aid in HCC detection, which overcome the disadvantages of the μTASWako AFP-L3.

Accordingly, in a seventh aspect, the invention relates to the use of the antibody or antigen binding fragment of the first aspect for an in vitro immunoassay, in particular an in vitro immunoassay for detecting α-1,6-core-fucosylated alpha-fetoprotein (AFP) or AFP-L3.

Similarly, the eighth aspect of the invention relates to an in vitro immunoassay method for detecting α-1,6-core-fucosylated AFP or a partial AFP sequence comprising said α-1,6-core-fucosylation in a sample using the monoclonal antibody or antigen binding fragment thereof according the first aspect.

The present inventors have also found that a pretreatment (e.g. using a reducing agent) can improve the signal obtained in such immunoassay suggesting that the epitope recognized by the antibodies and antigen binding fragments provided herein gets better accessible upon such pretreatment.

Accordingly, in a ninth aspect herein also provided is a pretreatment agent or pretreatment composition comprising a reducing agent (e.g. DTT).

In a tenth aspect, the present invention provides a kit of parts, comprising the monoclonal antibody or antigen binding fragment of the first aspect of the invention. In embodiments, the kit is for an immunoassay for detection and/or quantification of α-1,6-core-fucosylated AFP. In embodiments, the kit further comprises the pretreatment agent or composition of the ninth aspect of the invention.

### DETAILED DESCRIPTION OF THE INVENTION

In the following, the elements of the present invention will be described. These elements are listed with specific aspects and embodiments, however, it should be understood that they may be combined in any manner and in any number to create additional aspect and embodiments.

In a first aspect, the present disclosure relates to a monoclonal antibody or antigen binding fragment thereof specifically binding to α-1,6-core-fucosylated alpha-fetoprotein (AFP) or a partial sequence of AFP comprising said α-1,6-core-fucosylation.

As is known in the art, human AFP is a glycoprotein having the amino acid sequence SEQ ID NO: 1 (or natural occurring variants thereof as described in Uniprot ID P02771) comprising a single N-glycosylation site corresponding to Asn-251 of Uniprot ID P02771 (version 209). The N-glycan at Asn-251 can comprise a core fucose residue (see FIG. 1). As further understood in the art, the term “core fucosylation” within the glycan indicates that a fucose residue is α-1,6-linked to the core GlcNac residue attached to the Asn-251 of the AFP protein or partial sequence thereof comprising an Asn corresponding to Asn-251. The terms “core-fucosylation” and “α-1,6-core-fucosylation” are recognized to be interchangeable. Accordingly, the terms “specific for (or specifically binding to) core-fucosylated AFP and/or partial sequences of AFP comprising the core-fucosylation” are interchangeable with the terms “specific for (or specifically binding to) α-1,6-core-fucosylated AFP and partial sequences thereof comprising the α-1,6-core-fucosylation”. The antibodies and antibody antigen binding fragments of the invention are also interchangeably referenced herein as 1,6fucAFP antibodies and antigen binding fragments thereof.

As mentioned above, the α-1,6-core-fucose referenced to in the context of 1,6fucAFP antibodies or antigen binding fragments is part of an N-glycan attached to residue N251 of human AFP (see SEQ ID NO: 1).

The position of the α-1,6-core-fucose in the N-glycan is illustrated in FIG. 1.

The partial sequence of AFP may comprises SEQ ID NO: 2. In specific embodiments, the peptide part of the partial sequence of AFP consists of SEQ ID NO: 2.

As demonstrated in the appended Examples, the present inventors have identified two monoclonal antibodies (19B12 and 3C5) that are characterized by a high specificity for α-1,6-core-fucosylated AFP (i.e. the component characterizing AFP-L3). The monoclonal antibodies bind in an α-1,6-core-fucose dependent manner but also the peptide part of AFP contributes to the binding of α-1,6-core-fucosylated AFP. Thus the monoclonal antibodies and antigen binding fragments thereof identified by the inventors discriminate α-1,6-core-fucosylated AFP from different AFP species lacking the α-1,6-core-fucosylation at Asn-251 but also other proteins modified with an α-1,6-core-fucosylated N-glycan.

The monoclonal antibodies and antigen-fragments of the invention show a particularly high affinity (i.e. low K_{D}). This was somewhat unexpected and surprising based on previously reported antibodies against α-1,6-core-fucosylated AFP (Egashira Y et al., Scientific Reports, 2019, 9:12359). Moreover, the monoclonal antibodies of the invention show favorable kinetic parameters for use in a fully automated high throughput immunoassay, such as a high association rate constant kₐ and a low dissociation rate constant k_{d}.

As specified above, the monoclonal 1,6fucAFP antibody or antigen binding fragment provided herein specifically binds to α-1,6-core-fucosylated AFP or a partial sequence of AFP comprising said α-1,6-core-fucosylation. The α-1,6-core-fucosylated AFP or the partial sequence of AFP comprising said α-1,6-core-fucosylation may comprise or consist of the glycopeptide of Formula I

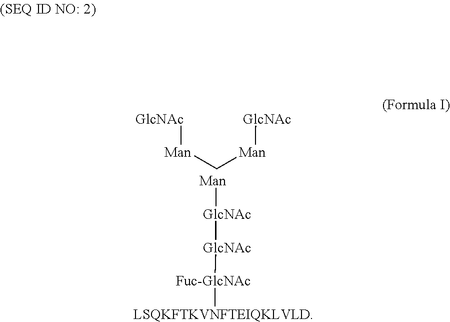

The glycopeptide of Formula I represents an AFP peptide with the amino acid sequence of SEQ ID NO: 2 comprising in position 3 an Asn amino acid residue corresponding to Asn-251 of AFP, said AFP peptide being modified with an N-glycan comprising an α-1,6-core-fucosylation.

Accordingly, the 1,6fucAFP antibodies of the invention and their antigen binding fragments may specifically bind the glycopeptide of Formula I, or glycoproteins comprising the glycopeptide of Formula I.

In embodiments, the α-1,6-core-fucosylated AFP or the partial sequence of AFP comprising said α-1,6-core-fucosylation specifically bound by the monoclonal 1,6fucAFP antibody or antigen binding fragment provided herein may comprise or consist of the glycopeptide of Formula II

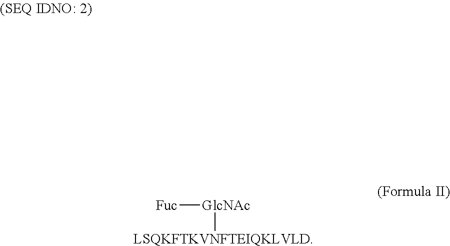

The glycopeptide of Formula II represents an AFP peptide with the amino acid sequence of SEQ ID NO: 2 comprising in position 3 an Asn amino acid residue corresponding to Asn-251 of AFP, said AFP peptide being modified with an N-glycan comprising an α-1,6-core-fucosylation.

In the embodiments in which the α-1,6-core-fucosylated AFP or the partial sequence of AFP comprising said α-1,6-core-fucosylation comprise the glycopeptide of Formula II, for example, further amino acids may be present in the peptide sequence or further sugar moieties may be present in the glycan (preferably attached to the GlcNAc).

Accordingly, the 1,6fucAFP antibodies of the invention and their antigen binding fragments may specifically bind the glycopeptide of Formula II, or glycoproteins comprising the glycopeptide of Formula II.

In embodiments, the 1,6fucAFP antibodies of the invention and their antigen binding fragments may specifically bind the glycopeptide of Formula I or II, or glycoproteins comprising the glycopeptide of Formula I or II.

As demonstrated in the appended Examples the 1,6fucAFP antibodies and antigen binding fragments of the present disclosure bind to both the glycopeptide of Formula I and the glycopeptide of Formula II with a high affinity (i.e. low K_{D}). What has been surprisingly observed is that the binding affinity to the glycopeptide of Formula I is slightly higher (i.e. the K_{D} is lower) than for the binding to glycopeptide of Formula II. This indicates that the addition sugar moieties in the N-glycan of the glycopeptide of Formula I somewhat foster the binding. As in nature N-glycan structures are typically more complex, such as the one shown in the glycopeptide of Formula I, this binding behavior may be advantageous in that it increases specificity towards more complex N-glycosylated AFP species.

Accordingly, in embodiments the 1,6fucAFP antibodies of the invention and their antigen binding fragments may bind to the glycopeptide of Formula I with a higher affinity (i.e. lower K_{D}) than to the glycopeptide of Formula II. In embodiments the ratio between the K_{D} for the binding to glycopeptide of Formula II and the glycopeptide of Formula I may be at least 2, at least 3, at least 4, at least 5, at least 6, at least 7 or at least 10. In a particular embodiment the ratio between the K_{D} for the binding to glycopeptide of Formula II and the glycopeptide of Formula I may be at least 7. In embodiments, the ratio between the K_{D} for the binding to glycopeptide of Formula II and the glycopeptide of Formula I may be at most 11, at most 12 or at most 15. In embodiments, the ratio between the K_{D} for the binding to glycopeptide of Formula II and the glycopeptide of Formula I may be at most 12.

As already discussed above, the 1,6fucAFP antibodies of the invention and their antigen binding fragments discriminate between (i) the α-1,6-core-fucosylated AFP or the partial sequence of AFP comprising said α-1,6-core-fucosylation (e.g. of Formula I) and (ii) AFP or a partial sequence thereof lacking the α-1,6-core-fucose residue.

AFP or a partial sequence thereof lacking the α-1,6-core-fucose residue may include (i) AFP or a partial sequence thereof being N-glycosylated at Asn-251 or the position corresponding thereto but lacking a core fucose in the N-glycan and (ii) aglycosylated AFP or a partial sequence thereof.

Furthermore, the 1,6fucAFP antibodies of the invention and their antigen binding fragments discriminate between (i) the α-1,6-core-fucosylated AFP or the partial sequence of AFP comprising said α-1,6-core-fucosylation (e.g. of Formula I) and (ii) an α-1,6-core-fucosylated N-glycan in isolation or in presence of a protein other than AFP.

As also defined herein below, the term “discriminates” indicates that the 1,6fucAFP antibodies and antigen binding fragments bind the specific antigenic target (i.e. core fucosylated AFP and core-fucosylated partial sequences thereof) with greater affinity and/or specificity than they bind other antigens (“non-target antigens”), e.g. AFP/AFP partial sequences lacking the core fucose residue and/or the core-fucosylated glycan, e.g. in isolated form or in the context of other glycosylated proteins or peptides. For example, the feature of discriminating a target antigen from/over a non-target antigen may be characterized by the 1,6fucAFP antibody or antibody antigen binding fragment having an affinity for the target antigen that is at least 10 fold, at least 20 fold, at least 50 fold or at least 100 fold, at least 150 fold, at least 180 fold or at least 245 fold better than the affinity for the non-target antigen (i.e. the K_{D} for the binding to the target antigen is at least 10 fold, at least 20 fold, at least 50 fold or at least 100 fold, at least 150 fold, at least 180 fold or at least 245 fold lower than for the binding to the non-target antigen). The formulation at least “XX” also covers the embodiment, that the K_{D} for the non-target antigen is so high that it cannot be detected with the method used. Accordingly, whether an antibody or antigen binding fragment can discriminate the target structure from a non-target structure may be determining the K_{D} values for the respective bindings using the same method (e.g., the preferred method for determining the K_{D} as described herein below.

In embodiments, the capability of an antibody to discriminate between the target antigen from/over a non-target antigen can be assessed using an immunoassay in which the binding of the to be tested antibody or antigen-binding fragment to a target structure is detected. Using such immunoassay the immunoassay signal obtained with a first sample comprising the target antigen in a defined concentration can be compared with the immunoassay signal obtained with a second sample comprising the non-target antigen in the same concentration. An immunoassay signal of the first sample being higher than the immunoassay signal from the second sample indicates a discrimination between the target antigen and non-target antigen. In embodiments, the tested antibody or antigen-binding fragment may discriminate between the target structure and the non-target structure if the immunoassay signal of the first sample is at least 5-fold, at least 10 fold, at least 20 fold, at least 40 fold, at least 50 fold, or at least 100 higher than for the second sample. An exemplary but non-limiting immunoassay that can be used for such analysis is provided in Example 7. An exemplary concentration of the target antigen and non-target antigen may be 12 nM.

In embodiments, AFP or a partial sequence thereof lacking the α-1,6-core-fucose residue may comprise or consist of the glycopeptide of Formula III,

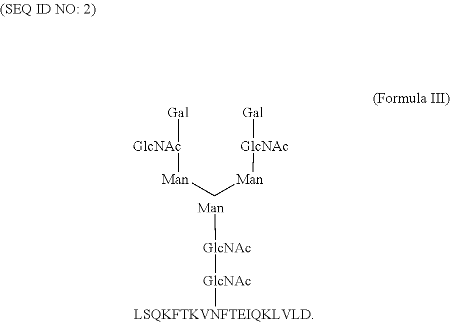

As Formula III lacks the core-fucose, the 1,6fucAFP antibodies or their antigen binding fragments can discriminate this the glycopeptide of Formula I or a glycoprotein comprising the same from the glycopeptide of Formula III.

Accordingly, provided herein are 1,6fucAFP antibodies and antigen binding-fragments thereof, which discriminate between the glycopeptide of Formula I (or an AFP sequence comprising the same) and a glycopeptide of Formula III (or an AFP sequence comprising the same).

In embodiments, the 1,6fucAFP antibodies and antigen binding-fragments of the invention discriminate between the glycopeptide of Formula II (or an AFP sequence comprising the same) and a glycopeptide of Formula III (or an AFP sequence comprising the same).

In embodiments, the 1,6fucAFP antibodies and antigen binding-fragments of the invention discriminate between the glycopeptide of Formula I (or an AFP sequence comprising the same) and a peptide of SEQ ID NO: 2 or SEQ ID NO:25 (or an AFP sequence comprising the same).

In embodiments, the 1,6fucAFP antibodies and antigen binding-fragments of the invention discriminate between the glycopeptide of Formula II (or an AFP sequence comprising the same) and a peptide of SEQ ID NO: 2 or SEQ ID NO:25 (or an AFP sequence comprising the same).

In embodiments, the 1,6fucAFP antibodies and antigen binding-fragments provided herein discriminate between the glycopeptide of Formula I (or an AFP sequence comprising the same) and both a glycopeptide of Formula III (or an AFP sequence comprising the same) and a peptide of SEQ ID NO: 2 or SEQ ID NO:25 (or an AFP sequence comprising the same).

In embodiments, the 1,6fucAFP antibodies and antigen binding-fragments of the invention discriminate between the glycopeptide of Formula II (or an AFP sequence comprising the same) and both a glycopeptide of Formula III (or an AFP sequence comprising the same) and a peptide of SEQ ID NO:25 (or an AFP sequence comprising the same).

In embodiments, the 1,6fucAFP antibodies and antigen binding-fragments of the invention binds to (i) α-1,6-core-fucosylated AFP or the partial AFP sequence comprising the α-1,6-core-fucosylation (e.g. glycopeptide of formula I) with an equilibrium dissociation constant (K_{D}) that is at least 10 fold, at least 20 fold, at least 50 fold or at least 100 fold, at least 150 fold, at least 180 fold, at least 183 fold or at least 245 fold lower than the K_{D} for the binding to (ii) AFP lacking the α-1,6-core-fucose residue or the partial sequence of AFP lacking the α-1,6-core-fucose residue (e.g. glycopeptide of Formula III or SEQ ID NO: 2 or 25). In a specific embodiment, said difference in the K_{D} is at least 100 fold. In another specific embodiment, said difference in the dissociation constant is at least 180 fold. In yet another specific embodiment, said difference in the dissociation is at least 183 fold. The binding affinity for (i) and (ii) are determined under the same conditions.

In embodiments, the AFP lacking the α-1,6-core-fucose residue or the partial sequence of AFP lacking the α-1,6-core-fucose residue is N-glycosylated AFP or a partial sequence thereof lacking the core fucose residue (e.g. glycopeptide of formula III); and the 1,6fucAFP antibodies and antigen binding-fragments of the invention binds to (i) α-1,6-core-fucosylated AFP or the partial AFP sequence (e.g. SEQ ID NO:2) comprising the α-1,6-core-fucosylation with an equilibrium dissociation constant (K_{D}) that is at least 10 fold, at least 20 fold, at least 50 fold or at least 100 fold, at least 150 fold, at least 180 fold, at least 183 fold or at least 245 fold lower than the K_{D} for the binding to (ii) AFP lacking the α-1,6-core-fucose residue or the partial sequence of AFP lacking the α-1,6-core-fucose residue. In a specific embodiment, said difference in the K_{D} is at least 100 fold. In another specific embodiment, said difference in the dissociation constant is at least 180 fold. In yet another specific embodiment, said difference in the dissociation is at least 183 fold. The binding affinity for (i) and (ii) are determined under the same conditions.

In embodiments, the 1,6fucAFP antibodies and antigen binding-fragments of the invention binds to (i) the glycopeptide of Formula I with an equilibrium dissociation constant (K_{D}) that is at least 10 fold, at least 20 fold, at least 50 fold or at least 100 fold, at least 150 fold, at least 180 fold, at least 183 fold or at least 245 fold lower than the K_{D} for the binding of the antibodies or antigen binding fragments to (ii) the glycopeptide of formula III. In a specific embodiment, said difference in the K_{D} is at least 100 fold. In another specific embodiment, said difference in the dissociation constant is at least 180 fold. In yet another specific embodiment, said difference in the dissociation is at least 183 fold. The binding affinity for (i) and (ii) are determined under the same conditions.

In embodiments, the AFP lacking the α-1,6-core-fucose residue or the partial sequence of AFP lacking the α-1,6-core-fucose residue is aglycosylated AFP or an aglycosylated partial AFP sequence (e.g. SEQ ID NO: 2 or 25) and the 1,6fucAFP antibodies and antigen binding-fragments of the invention binds to (i) α-1,6-core-fucosylated AFP or the partial AFP sequence comprising the α-1,6-core-fucosylation (e.g. glycopeptide of formula I) with an equilibrium dissociation constant (K_{D}) that is at least 10 fold, at least 20 fold, at least 50 fold or at least 100 fold, at least 150 fold, at least 180 fold, at least 183 fold or at least 245 fold, at least 500 fold, at least 1000 fold, at least 3000 fold, or at least 5000 fold lower than the K_{D} for the binding to said aglycosylated AFP or aglycosylated partial AFP sequence (e.g. SEQ ID NO: 2 or 25). In embodiments, said difference in the K_{D} is at least 1000 fold. In embodiments, said difference in the K_{D} is at least 5000 fold. The binding affinity for (i) and (ii) are determined under the same conditions.

In embodiments, the 1,6fucAFP antibodies and antigen binding-fragments of the invention binds to (i) the glycopeptide of Formula I with an equilibrium dissociation constant (K_{D}) that is at least 10 fold, at least 20 fold, at least 50 fold or at least 100 fold, at least 150 fold, at least 180 fold, at least 183 fold or at least 245 fold, at least 500 fold, at least 1000 fold, at least 3000 fold, or at least 5000 fold lower than the K_{D} for the binding of the antibodies or antigen binding fragments to (ii) the AFP peptide of SEQ ID NO: 25. In embodiments, said difference in the K_{D} is at least 1000 fold. In embodiments, said difference in the K_{D} is at least 4000 fold. In embodiments, said difference in the K_{D} is at least 5000 fold. The binding affinity for (i) and (ii) are determined under the same conditions.

As demonstrated with respective glycopeptides and glycans in the appended Examples and Figures, the monoclonal antibodies or antigen binding fragments of the present invention discriminate between (i) α-1,6-core-fucosylated AFP or a partial sequence of AFP comprising said α-1,6-core-fucosylation (e.g. glycopeptide of formula I), and (ii) an isolated α-1,6-core-fucosylated glycan (e.g. of formula (IV)) and other glycoproteins with an α-1,6-core-fucosylated glycan (i.e. proteins that comprise the glycopeptide of formula IV but do not comprise the glycopeptide of formula I).

The glycan of Formula IV has the following structure.

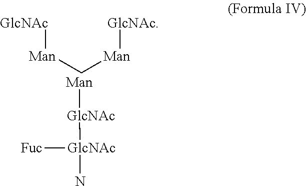

Accordingly, in embodiments, the 1,6fucAFP monoclonal antibodies or antigen binding fragments of the present invention discriminate between (i) α-1,6-core-fucosylated AFP or the partial sequence of AFP comprising said α-1,6-core-fucosylation (e.g. glycopeptide of formula I), and (ii) an isolated α-1,6-core-fucosylated glycan (e.g. of formula (IV)) and other glycoproteins with an α-1,6-core-fucosylated glycan (i.e. proteins that comprise the glycopeptide of formula IV but do not comprise the glycopeptide of formula I).

In particular embodiments, the 1,6fucAFP monoclonal antibodies or antigen binding fragments of the present invention bind to (i) α-1,6-core-fucosylated AFP or a partial AFP sequence comprising the α-1,6-core-fucosylation (e.g. glycopeptide of formula I) with an equilibrium dissociation constant (K_{D}) that is at least 10 fold, at least 20 fold, at least 50 fold or at least 100 fold, at least 150 fold, at least 180 fold, at least 245 fold lower than the K_{D} for the binding of the antibody or antigen binding fragment to (ii) an isolated α-1,6-core-fucosylated glycan (e.g. of formula (IV)) and other glycoproteins with an α-1,6-core-fucosylated glycan (i.e. proteins that comprise the glycopeptide of formula IV but do not comprise the glycopeptide of formula I). The binding affinity for (i) and (ii) are determined under the same conditions. In a specific embodiment, said difference in the K_{D} is at least 100 fold. In another specific embodiment, said difference in the dissociation constant is at least 180 fold. In yet another specific embodiment, said difference in the dissociation is at least 183 fold. The binding affinity for (i) and (ii) are determined under the same conditions.

In an even more specific embodiment, the 1,6fucAFP monoclonal antibodies or antigen binding fragments of the present invention bind to (i) the glycopeptide of formula I with an equilibrium dissociation constant (K_{D}) that is at least 10 fold, at least 20 fold, at least 50 fold or at least 100 fold, at least 150 fold, at least 180 fold, at least 245 fold lower than the K_{D} for the binding of the antibody or antigen binding fragment to (ii) the isolated α-1,6-core-fucosylated glycan of formula (IV). The binding affinity for (i) and (ii) are determined under the same conditions. In a specific embodiment, said difference in the K_{D} is at least 100 fold. In another specific embodiment, said difference in the dissociation constant is at least 180 fold. In yet another specific embodiment, said difference in the dissociation is at least 183 fold. The binding affinity for (i) and (ii) are determined under the same conditions.

In embodiments, the 1,6fucAFP monoclonal antibodies or antigen binding fragments of the present invention discriminate between the glycopeptide according to Formula I and both the glycopeptide of Formula III and the core-fucosylated glycan of Formula IV. What has been said with respect to the fold differences in K_{D} values between the individual structure pairs mentioned herein elsewhere (e.g. above) applies mutatis mutandis.

In embodiments, the 1,6fucAFP monoclonal antibodies or antigen binding fragments of the present invention discriminate between the glycopeptide according to Formula I and all of the following three: the glycopeptide of Formula III, the core-fucosylated glycan of Formula IV and the AFP peptide of SEQ ID NO: 25 or 2 (e.g. SEQ ID NO: 25).

The monoclonal 1,6fucAFP monoclonal antibodies or antigen binding fragments of the present invention have been found to have a surprisingly high affinity (i.e. low K_{D}) towards the glycopeptide of formula I (see above), i.e. to a AFP sequence comprising the α-1,6-core-fucosylation. A low K_{D} is typically advantageous for immunoassays.

In embodiments, the monoclonal 1,6fucAFP monoclonal antibodies or antigen binding fragments of the present invention bind to the glycopeptide of Formula I with a K_{D} of 100 nM or less, 20 nM or less, preferably 10 nM or less, more preferably 3.1 nM or less or 2.5 nM or less. If the K_{D} is determined by affinity in solution, the K_{D} may also be 0.9 nM or less or 0.4 nM or less. In specific embodiments, the monoclonal 1,6fucAFP monoclonal antibodies or antigen binding fragments of the present invention bind to the glycopeptide of Formula I with a K_{D} of 20 nM or less. In specific embodiments, the monoclonal 1,6fucAFP monoclonal antibodies or antigen binding fragments of the present invention bind to the glycopeptide of Formula I with a K_{D} of 10 nM or less. In specific embodiments, the monoclonal 1,6fucAFP monoclonal antibodies or antigen binding fragments of the present invention bind to the glycopeptide of Formula I with a K_{D} of 5 nM or less.

In embodiments, the monoclonal 1,6fucAFP monoclonal antibodies or antigen binding fragments of the present invention bind to the glycopeptide of Formula I with a K_{D} that is less than 10, 8, 6, 4, or 2 times the K_{D} of a rabbit IgG antibody comprising the heavy chain variable domain of SEQ ID NO: 11 and a light chain variable domain of SEQ ID NO: 13 (e.g. antibody 19B12) towards the glycopeptide of Formula I, wherein the K_{D} values are measured under the same conditions and using the same method. In specific embodiments, the monoclonal 1,6fucAFP monoclonal antibodies or antigen binding fragments of the present invention bind to the glycopeptide of Formula I with a K_{D} that is equal or less than the K_{D} of a rabbit IgG antibody comprising the heavy chain variable domain of SEQ ID NO: 11 and a light chain variable domain of SEQ ID NO: 13 (e.g. antibody 19B12) towards the glycopeptide of Formula I, wherein the K_{D} values are measured under the same conditions and using the same method.

In embodiments, the monoclonal 1,6fucAFP monoclonal antibodies or antigen binding fragments of the present invention bind to the glycopeptide of Formula I with a K_{D} that is less than 10, 8, 6, 4, or 2 times the K_{D} of a rabbit IgG antibody comprising the heavy chain variable domain of SEQ ID NO: 12 and a light chain variable domain of SEQ ID NO: 14 (e.g. antibody 3C5) towards the glycopeptide of Formula I, wherein the K_{D} values are measured under the same conditions and using the same method. In specific embodiments, the monoclonal 1,6fucAFP monoclonal antibodies or antigen binding fragments of the present invention bind to the glycopeptide of Formula I with a K_{D} that is equal or less than the K_{D} of a rabbit IgG antibody comprising the heavy chain variable domain of SEQ ID NO: 12 and a light chain variable domain of SEQ ID NO: 14 (e.g. antibody 3C5) towards the glycopeptide of Formula I, wherein the K_{D} values are measured under the same conditions and using the same method.

The monoclonal 1,6fucAFP monoclonal antibodies or antigen binding fragments of the present invention have further been found to have a surprisingly high association rate (kₐ) constant for the binding to the glycopeptide of formula I (see above), i.e. to a AFP sequence comprising the α-1,6-core-fucosylation. A high kₐ is important for a fast binding of an antibody to the antigen in an equilibrium. Consequently, a high kₐ is important for low incubation times in immunoassays.

The monoclonal 1,6fucAFP monoclonal antibodies or antigen binding fragments of the present invention may have an association rate constant (kₐ) for the binding to the glycopeptide of Formula I, which is at least 2.0*10⁴ M⁻¹s⁻¹, in embodiments at least 2.1*10⁴ M⁻¹s⁻¹, in embodiments at least 10⁵ M⁻¹s⁻¹, in embodiments at least 2.5*10⁵ M⁻¹s⁻¹, in embodiments at least 6.0*10⁵ M⁻¹s⁻¹ and in embodiments at least 1.0*10⁶ M⁻¹s⁻¹.

In embodiments, the monoclonal 1,6fucAFP monoclonal antibodies or antigen binding fragments of the present invention bind to the glycopeptide of Formula I with a kₐ that is higher than 3.4%, 5%, 10%, 20%, 50%, 80% or 90% of the kₐ of a rabbit IgG antibody comprising the heavy chain variable domain of SEQ ID NO: 11 and a light chain variable domain of SEQ ID NO: 13 (e.g. antibody 19B12) towards the glycopeptide of Formula I, wherein the kₐ values are measured under the same conditions and using the same method. In specific embodiments, the monoclonal 1,6fucAFP monoclonal antibodies or antigen binding fragments of the present invention bind to the glycopeptide of Formula I with a kₐ that is equal or higher than the kₐ of a rabbit IgG antibody comprising the heavy chain variable domain of SEQ ID NO: 11 and a light chain variable domain of SEQ ID NO: 13 (e.g. antibody 19B12) towards the glycopeptide of Formula I, wherein the kₐ values are measured under the same conditions and using the same method.

In embodiments, the monoclonal 1,6fucAFP monoclonal antibodies or antigen binding fragments of the present invention bind to the glycopeptide of Formula I with a kₐ that is higher than 2.0%, 3.4%, 5%, 10%, 20%, 50%, 80% or 90% of the kₐ of a rabbit IgG antibody comprising the heavy chain variable domain of SEQ ID NO: 12 and a light chain variable domain of SEQ ID NO: 14 (e.g. antibody 3C5) towards the glycopeptide of Formula I, wherein the kₐ values are measured under the same conditions and using the same method. In specific embodiments, the monoclonal 1,6fucAFP monoclonal antibodies or antigen binding fragments of the present invention bind to the glycopeptide of Formula I with a kₐ that is equal to or higher than the kₐ of a rabbit IgG antibody comprising the heavy chain variable domain of SEQ ID NO: 12 and a light chain variable domain of SEQ ID NO: 14 (e.g. antibody 3C5) towards the glycopeptide of Formula I, wherein the kₐ values are measured under the same conditions and using the same method.

The monoclonal 1,6fucAFP monoclonal antibodies or antigen binding fragments of the present invention have further been found to have a surprisingly low dissociation rate (k_{d}) constant for the binding to the glycopeptide of formula I (see above), i.e. to a AFP sequence comprising the α-1,6-core-fucosylation.

The monoclonal 1,6fucAFP monoclonal antibodies or antigen binding fragments of the present invention may have a dissociation rate constant (k_{d}) for the glycopeptide of Formula I, which is at most 1.2*10⁻² s⁻¹, at most 1.3*10⁻² s⁻¹, at most *10⁻² s⁻¹, at most 8.0*10⁻³ s⁻¹, at most 7.3*10⁻³ s⁻¹, at most 3.2*10⁻³ s⁻¹ or at most 1.5*10⁻³ s⁻¹.

In embodiments, the monoclonal 1,6fucAFP monoclonal antibodies or antigen binding fragments of the present invention bind to the glycopeptide of Formula I with a k_{d} that is lower than 1.5, 2, 2.5, 3, 3.5 or 3.75 times the k_{d} of a rabbit IgG antibody comprising the heavy chain variable domain of SEQ ID NO: 11 and a light chain variable domain of SEQ ID NO: 13 (e.g. antibody 19B12) towards the glycopeptide of Formula I, wherein the k_{d} values are measured under the same conditions and using the same method. In specific embodiments, the monoclonal 1,6fucAFP monoclonal antibodies or antigen binding fragments of the present invention bind to the glycopeptide of Formula I with a k_{d} that is equal or lower than the k_{d} of a rabbit IgG antibody comprising the heavy chain variable domain of SEQ ID NO: 11 and a light chain variable domain of SEQ ID NO: 13 (e.g. antibody 19B12) towards the glycopeptide of Formula I, wherein the k_{d} values are measured under the same conditions and using the same method.

In embodiments, the monoclonal 1,6fucAFP monoclonal antibodies or antigen binding fragments of the present invention bind to the glycopeptide of Formula I with a k_{d} that is lower than 1.5, 2, 2.5, 3, 3.5, 3.75, 5, 6, 7, or 8 times the k_{d} of a rabbit IgG antibody comprising the heavy chain variable domain of SEQ ID NO: 12 and a light chain variable domain of SEQ ID NO: 14 (e.g. antibody 3C5) towards the glycopeptide of Formula I, wherein the k_{d} values are measured under the same conditions and using the same method. In specific embodiments, the monoclonal 1,6fucAFP monoclonal antibodies or antigen binding fragments of the present invention bind to the glycopeptide of Formula I with a k_{d} that is equal to or lower than the k_{d} of a rabbit IgG antibody comprising the heavy chain variable domain of SEQ ID NO: 12 and a light chain variable domain of SEQ ID NO: 14 (e.g. antibody 3C5) towards the glycopeptide of Formula I, wherein the k_{d} values are measured under the same conditions and using the same method.

In embodiments all K_{D}, kₐ and k_{d} values as disclosed herein may be K_{D}, kₐ and k_{d} values at 37° C.

As understood in the art, K_{D}, kₐ and k_{d} values may be determined by any conventional means known to the skilled person or as described herein.

In the context of the present invention the K_{D}, kₐ and k_{d} values mentioned herein, especially herein above are preferably determined by surface plasmon resonance spectroscopy (e.g. BIAcore®). Fold differences (i.e. ratios) between two K_{D} values, two kₐ values and two k_{d} values, respectively, can be calculated based on the two respective values measured by plasmon resonance spectroscopy. Preferred peptides and methods to perform the analyses are disclosed in the appended Examples and Figures.

It is particularly preferred herein to use the surface plasmon resonance spectroscopy methods as described in Example 3 to determine the K_{D}, kₐ and k_{d} values.

Accordingly, the surface plasmon resonance spectroscopy for determining K_{D}, kₐ and/or k_{d} values may comprise capturing the monoclonal antibody or antigen binding fragment on a C1 sensor chip (e.g. series S C1 sensor chip) and injecting the glycopeptide to be analyzed as analyte, wherein said determination is conducted at a temperature of 37° C. HBS-ET pH 7.4 (10 mM HEPES pH 7.4, 150 mM NaCl, 3 mM EDTA, 0.05% w/v Tween 20®) may be used as system buffer and said system buffer supplemented with 1 mg/ml carboxymethyldextran may be used as sample buffer.

In embodiments, the binding parameters K_{D} and/or kₐ and/or k_{d} may be determined by a Langmuir fit 1:1 fitting model, e.g. according to the BIAcore™ T200 Evaluation SW 3.2. Preferably, the fitting is with Rₘₐₓ global. Alternatively or additionally, the Langmuir 1:1 fitting Scrubber-SW V2.0c may be applied.

The surface plasmon resonance spectroscopy may be performed with different instruments known in the art. In a particularly preferred embodiment a BIAcore® T200 or 8K instrument may be used.

The concentration of the antibody or antigen-binding fragment used for the surface plasmon resonance measurement may be 150 nM and may be injected at, for example, 10 μl/min for 30 seconds. For the analyte (e.g. a peptide or glycopeptide) concentration series of 3.3-270 nm may be used. The injection of the analyte may be at 60 μl/min.

The association phase in the surface plasmon resonance spectroscopy measurement may, for example, be monitored for 3 minutes. The dissociation phase may, for example, be monitored for 10 minutes. The regeneration may be performed by an injection of 10 mM Glycine pH 2.0 at 20 μL/min for 30 seconds, followed by 2 injections of 10 mM Glycine pH 2.25 for 60 seconds.

In a preferred embodiment, the K_{D} and/or kₐ and/or k_{d} may be determined as follows:

A BIAcore® T200 instrument from GE Healthcare at 37° C. using a series S C1-sensor may be used. A rabbit antibody capture system (e.g. as described in the Examples) may be immobilized on the flow cells, e.g. with 700 to 800 RU. One flow cell may be used as control, three flow cells may be used for measurements. 150 nM of the to be tested antibody or antigen binding fragment may then be injected at 10 μL/min for 30 seconds. The Capture Levels (CL) in resonance units RU may be monitored. Analyte concentration series from 3.3-270 nM may be injected at 60 μl/min. The association phase may be monitored for 3 minutes, the dissociation phase may be monitored for 10 minutes. The regeneration may be performed by an injection of 10 mM Glycine pH 2.0 at 20 μL/min for 30 seconds, followed by 2 injections of 10 mM Glycine pH 2.25 for 60 seconds. The kinetic rate constants and the dissociation equilibrium constants K_{D} may determined using a Langmuir 1:1 fitting model according to the BIAcore™ T200 Evaluation SW 3.2. Secondly, the Langmuir 1:1 fitting Scrubber-SW V2.0c may be applied.

If not explicitly mentioned to the contrary herein, K_{D} values (and the rate constants) provided herein are determined with a surface plasmon resonance spectroscopy method using capturing the monoclonal antibody or antigen binding fragment on a chip (e.g. C1 sensor chip) and injecting the analyte to be analyzed. As well known in the art there are also alternative methods known in the art for determining K_{D} values and/or the rate constants. As long as the results obtained for the antibodies 3C5 and 19B12 with these methods concur with the results reported herein for the antibodies in Example 3 also such alternative methods may be used.

In the appended Examples also an alternative method for determining the K_{D} values is described: an affinity in solution analysis. As shown in the appended Examples the K_{D} values determined using affinity in solution analysis are very similar to capturing based methods as described above, but may differ to some degree. K_{D} values referenced to herein only relate to a K_{D} as measured by affinity in solution if explicitly referred to such technique. In case of discrepancy, an antibody capture based surface plasmon resonance spectroscopy data set as described above shall be used.

Affinity in solution measurements may be conducted as described following the vendors instructions for the CAP-Kit (Cytiva). For example, the biotinylated analyte (e.g. glycopeptide or peptide) may be captured on the CAP chip sensor surface. Mixtures of 10 nM antibody or antigen-binding fragment to be tested and varying concentrations between 120 nM and 0.01 nM of non-biotinylated AFP(243-261)-G0F resp. varying concentrations between 200 μM-0.1 nM of non-biotinylated AFP(248-256) may be incubated until equilibrium is achieved. Binding events of the mixtures to the surface displayed analyte may be monitored. With increasing peptide concentration as a competitor, the ‘free’ antibody in solution will decrease. The determined free antibody concentrations for the competition experiment may then be plotted versus the peptide competitor concentration. The Affinity in Solution model from BIAcore® Evaluation software may be used to evaluate the data and to determine the K_{D}.

In embodiments, the method for the determination of the K_{D} is selected such that the K_{D} of the binding between
(1) a rabbit IgG antibody comprising a heavy chain variable domain having the sequence of SEQ ID NO: 11 and a light chain variable domain having the sequence of SEQ ID NO: 13; and
(2) the glycopeptide of Formula I
   is determined to be 2.5 (error of method, e.g. 0.08%) nM.

In embodiments, the method for the determination of the K_{D} is selected such that the K_{D} of the binding between
(1) a rabbit IgG antibody comprising a heavy chain variable domain having the sequence of SEQ ID NO: 12 and a light chain variable domain having the sequence of SEQ ID NO: 14; and
(2) the glycopeptide of Formula I
   is determined to be 3.1 (±error of method, e.g. 0.13%) nM.

As demonstrated in the appended Examples and as further discussed herein below, the 1,6fucAFP antibodies or antigen binding fragments can detect α-1,6-core-fucosylated alpha-fetoprotein (AFP) (e.g. natural α-1,6-core-fucosylated as occurring in blood samples (e.g. plasma or serum) obtained from a human subject) that has been pretreated with a pretreatment agent according to the present invention (see herein elsewhere) much better than without such pretreatment. Accordingly, in embodiments, the monoclonal antibodies or antigen binding fragments of the present disclosure bind to α-1,6-core-fucosylated alpha-fetoprotein (AFP) better than to α-1,6-core-fucosylated alpha-fetoprotein (AFP) without such pretreatment. A measure to detect such “better” binding may the signal using one of the immunoassay set-ups as disclosed in the appended Examples. The pretreatment agent is preferably a reducing agent, such as DTT.

Surprisingly, the two best monoclonal 1,6fucAFP antibodies identified herein (i.e. 19B12 and 3C5), show a very high sequence homology, yet with certain differences in the framework regions but also the CDR residues. This demonstrates that the CDR sequences as well as VH and VL sequences underlying the identified antibodies define very valuable building blocks for generating 1,6fucAFP antibodies and antigen binding fragments thereof. However, the sequence variability found in the VHs and VLs also demonstrates that certain amino acid exchanges can be made in both the CDRs and framework regions of the VH and VL without significantly negatively affecting the antibodies properties. As will be appreciated, a skilled person could generate easily variants of the antibody or antigen binding fragments sequences of the invention and could determine based on the disclosure herein whether such antibodies maintain the advantage properties disclosed herein. For example, affinity maturation may be applied such as to obtain variant monoclonal antibodies or antigen binding fragments comprising the VH and VL of 19B12 or 3C5 with amino acid substitutions as obtained by such affinity maturation that have the same or improved properties than 19B12 or 3C5.

In embodiments, the monoclonal antibody or antigen binding fragment provided herein comprises
(i) a heavy chain variable domain (VH) comprising a CDR-H1 having the amino acid sequence of SEQ ID NO: 3 (CDR-H1 of 19B12 and 3C5) or a variant thereof modified by the substitution, insertion or deletion of one amino acid; a CDR-H2 having the amino acid sequence of SEQ ID NO: 4 or 5 (CDR-H2 of 19B12 and 3C5, respectively) or a variant of SEQ ID NO: 4 or 5 modified by the substitution, insertion or deletion of at most two amino acids; and a CDR-H3 having the amino acid sequence of SEQ ID NO: 6 (CDR-H3 of 19B12 and 3C5) or a variant thereof modified by the substitution, insertion or deletion of one amino acid; and
(ii) a light chain variable domain (VL) comprising a CDR-L1 having the amino acid sequence of SEQ ID NO: 7 or 8 (CDR-L1 of 19B12 and 3C5, respectively) or a variant of SEQ ID NO: 7 or 8 modified by the substitution, insertion or deletion of at most two amino acids; a CDR-L2 having the amino acid sequence of SEQ ID NO: 9 (CDR-L2 of 19B12 and 3C5) or a variant thereof modified by the substitution, insertion or deletion of one amino acid; and a CDR-L3 having the amino acid sequence of SEQ ID NO: 10 (CDR-L3 of 19B12 and 3C5) or a variant thereof modified by the substitution, insertion or deletion of one amino acid.

In specific embodiments, the monoclonal antibody or antigen binding fragment provided herein comprises
(i) a heavy chain variable domain (VH) comprising a CDR-H1 having the amino acid sequence of SEQ ID NO: 3 (CDR-H1 of 19B12 and 3C5); a CDR-H2 having the amino acid sequence of SEQ ID NO: 4 or 5 (CDR-H2 of 19B12 and 3C5, respectively) or a variant of SEQ ID NO: 4 or 5 modified by the substitution of one amino acid and/or the insertion or deletion of one amino acid and a CDR-H3 having the amino acid sequence of SEQ ID NO: 6 (CDR-H3 of 19B12 and 3C5); and
(ii) a light chain variable domain (VL) comprising a CDR-L1 having the amino acid sequence of SEQ ID NO: 7 or 8 (CDR-L1 of 19B12 and 3C5, respectively) or a variant of SEQ ID NO: 7 or 8 modified by the substitution, insertion or deletion of one amino acid; a CDR-L2 having the amino acid sequence of SEQ ID NO: 9 (CDR-L2 of 19B12 and 3C5); and a CDR-L3 having the amino acid sequence of SEQ ID NO: 10 (CDR-L3 of 19B12 and 3C5).

In specific embodiments, the monoclonal antibody or antigen binding fragment provided herein comprises
(i) a heavy chain variable domain (VH) comprising a CDR-H1 consisting of the amino acid sequence of SEQ ID NO: 3 (CDR-H1 of 19B12 and 3C5); a CDR-H2 consisting of the amino acid sequence of SEQ ID NO: 4 or 5 (CDR-H2 of 19B12 and 3C5, respectively) or a variant of SEQ ID NO: 4 or 5 modified by the substitution of one amino acid and/or the insertion or deletion of one amino acid and a CDR-H3 consisting of the amino acid sequence of SEQ ID NO: 6 (CDR-H3 of 19B12 and 3C5); and
(ii) a light chain variable domain (VL) comprising a CDR-L1 consisting of the amino acid sequence of SEQ ID NO: 7 or 8 (CDR-L1 of 19B12 and 3C5, respectively) or a variant of SEQ ID NO: 7 or 8 modified by the substitution, insertion or deletion of one amino acid; a CDR-L2 consisting of the amino acid sequence of SEQ ID NO: 9 (CDR-L2 of 19B12 and 3C5); and a CDR-L3 consisting of the amino acid sequence of SEQ ID NO: 10 (CDR-L3 of 19B12 and 3C5).

In embodiments, the monoclonal antibody or antigen binding fragment provided herein comprises
(i) a heavy chain variable domain (VH) comprising a CDR-H1 consisting of the amino acid sequence of SEQ ID NO: 3 (CDR-H1 of 19B12 and 3C5) or a variant thereof modified by the substitution, insertion or deletion of one amino acid; a CDR-H2 consisting of the amino acid sequence of SEQ ID NO: 4 or 5 (CDR-H2 of 19B12 and 3C5, respectively) or a variant of SEQ ID NO: 4 or 5 modified by the substitution, insertion or deletion of at most two amino acids; and a CDR-H3 consisting of the amino acid sequence of SEQ ID NO: 6 (CDR-H3 of 19B12 and 3C5) or a variant thereof modified by the substitution, insertion or deletion of one amino acid; and
(ii) a light chain variable domain (VL) comprising a CDR-L1 consisting of the amino acid sequence of SEQ ID NO: 7 or 8 (CDR-L1 of 19B12 and 3C5, respectively) or a variant of SEQ ID NO: 7 or 8 modified by the substitution, insertion or deletion of at most two amino acids; a CDR-L2 consisting of the amino acid sequence of SEQ ID NO: 9 (CDR-L2 of 19B12 and 3C5) or a variant thereof modified by the substitution, insertion or deletion of one amino acid; and a CDR-L3 consisting of the amino acid sequence of SEQ ID NO: 10 (CDR-L3 of 19B12 and 3C5) or a variant thereof modified by the substitution, insertion or deletion of one amino acid.

The variation in the CDR sequences of the monoclonal antibody or antigen-fragment may be such that the total sum of the substitution, insertion and deletion events in all CDR sequences is at most 8, at most 7, at most 6, at most 5, at most 4, at most 3, or at most 2.

In a particular embodiment, the total sum of the amino acid substitutions, insertions and deletions in all CDR sequences may be at most 3. In embodiments, the at most 3 amino acid substitutions, insertions and deletion events may be at most two amino acid substitutions and one insertion or deletion.

In another particular embodiment, the total sum of the amino acid substitutions, insertions and deletions in all CDR sequences may be at most 2.

In embodiments, the monoclonal antibody or antigen binding fragment provided herein comprises
(i) a heavy chain variable domain (VH) comprising a CDR-H1 having the amino acid sequence of SEQ ID NO: 3 (CDR-H1 of 19B12 and 3C5) or a variant thereof modified by one amino acid substitution; a CDR-H2 having the amino acid sequence of SEQ ID NO: 4 or 5 (CDR-H2 of 19B12 and 3C5, respectively) or a variant of SEQ ID NO: 4 or 5 modified by at most two amino acid substitutions; and a CDR-H3 having the amino acid sequence of SEQ ID NO: 6 (CDR-H3 of 19B12 and 3C5) or a variant thereof modified by one amino acid substitution; and
(ii) a light chain variable domain (VL) comprising a CDR-L1 having the amino acid sequence of SEQ ID NO: 7 or 8 (CDR-L1 of 19B12 and 3C5, respectively) or a variant of SEQ ID NO: 7 or 8 modified by at most two amino acid substitutions; a CDR-L2 having the amino acid sequence of SEQ ID NO: 9 (CDR-L2 of 19B12 and 3C5) or a variant thereof modified by one amino acid substitution; and a CDR-L3 having the amino acid sequence of SEQ ID NO: 10 (CDR-L3 of 19B12 and 3C5) or a variant thereof modified by one amino acid substitution.

The expression “a variant thereof modified by one amino acid substitution” as used herein means that there is exactly one (not more than one) amino acid substitutions comprised in said variant sequence vis-à-vis the indicated parent sequence.

In specific embodiments, the monoclonal antibody or antigen binding fragment comprises
(i) a heavy chain variable domain (VH) comprising a CDR-H1 consisting of the amino acid sequence of SEQ ID NO: 3 or a variant thereof modified by one amino acid substitution; a CDR-H2 consisting of the amino acid sequence of SEQ ID NO: 4 or 5 or a variant of SEQ ID NO: 4 or 5 modified by at most two amino acid substitutions; and a CDR-H3 consisting of the amino acid sequence of SEQ ID NO: 6 or a variant thereof modified by one amino acid substitution; and
(ii) a light chain variable domain (VL) comprising a CDR-L1 consisting of the amino acid sequence of SEQ ID NO: 7 or 8 or a variant of SEQ ID NO: 7 or 8 modified by at most two amino acid substitutions; a CDR-L2 consisting of the amino acid sequence of SEQ ID NO: 9 or a variant thereof modified by one amino acid substitution; and a CDR-L3 consisting of the amino acid sequence of SEQ ID NO: 10 or a variant thereof modified by one amino acid substitution.

The variation in the CDR sequences of the monoclonal antibody or antigen-fragment may be such that the total sum of the amino acid substitutions in all CDR sequences is at most 8, at most 7, at most 6, at most 5, at most 4, at most 3, or at most 2.

In a particular embodiment, the total sum of the amino acid substitutions in all CDR sequences may be at most 3.

In another particular embodiment, the total sum of the amino acid substitutions in all CDR sequences may be at most 2.

In embodiments, the monoclonal antibody or antigen binding fragment provided herein comprises
(i) a heavy chain variable domain (VH) comprising a CDR-H1 having the amino acid sequence of SEQ ID NO: 3 (CDR-H1 of 19B12 and 3C5) or a variant thereof modified by one conservative amino acid substitution; a CDR-H2 having the amino acid sequence of SEQ ID NO: 4 or 5 (CDR-H2 of 19B12 and 3C5, respectively) or a variant of SEQ ID NO: 4 or 5 modified by at most two conservative amino acid substitutions; and a CDR-H3 having the amino acid sequence of SEQ ID NO: 6 (CDR-H3 of 19B12 and 3C5) or a variant thereof modified by one conservative amino acid substitution; and
(ii) a light chain variable domain (VL) comprising a CDR-L1 having the amino acid sequence of SEQ ID NO: 7 or 8 (CDR-L1 of 19B12 and 3C5, respectively) or a variant of SEQ ID NO: 7 or 8 modified by at most two conservative amino acid substitutions; a CDR-L2 having the amino acid sequence of SEQ ID NO: 9 (CDR-L2 of 19B12 and 3C5) or a variant thereof modified by one conservative amino acid substitution; and a CDR-L3 having the amino acid sequence of SEQ ID NO: 10 (CDR-L3 of 19B12 and 3C5) or a variant thereof modified by one conservative amino acid substitution.

In specific embodiments, the monoclonal antibody or antigen binding fragment comprises
(i) a heavy chain variable domain (VH) comprising a CDR-H1 consisting of the amino acid sequence of SEQ ID NO: 3 or a variant thereof modified by one conservative amino acid substitution; a CDR-H2 consisting of the amino acid sequence of SEQ ID NO: 4 or 5 or a variant of SEQ ID NO: 4 or 5 modified by at most two conservative amino acid substitutions; and a CDR-H3 consisting of the amino acid sequence of SEQ ID NO: 6 or a variant thereof modified by one conservative amino acid substitution; and
(ii) a light chain variable domain (VL) comprising a CDR-L1 consisting of the amino acid sequence of SEQ ID NO: 7 or 8 or a variant of SEQ ID NO: 7 or 8 modified by at most two conservative amino acid substitutions; a CDR-L2 consisting of the amino acid sequence of SEQ ID NO: 9 or a variant thereof modified by one conservative amino acid substitution; and a CDR-L3 consisting of the amino acid sequence of SEQ ID NO: 10 or a variant thereof modified by one conservative amino acid substitution.

In embodiments, the monoclonal antibody or antigen binding fragment provided herein comprises
(i) a heavy chain variable domain (VH) comprising a CDR-H1 having the amino acid sequence of SEQ ID NO: 3 (CDR-H1 of 19B12 and 3C5) or a variant thereof modified by one highly conservative amino acid substitution; a CDR-H2 having the amino acid sequence of SEQ ID NO: 4 or 5 (CDR-H2 of 19B12 and 3C5, respectively) or a variant of SEQ ID NO: 4 or 5 modified by at most two highly conservative amino acid substitutions; and a CDR-H3 having the amino acid sequence of SEQ ID NO: 6 (CDR-H3 of 19B12 and 3C5) or a variant thereof modified by one highly conservative amino acid substitution; and
(ii) a light chain variable domain (VL) comprising a CDR-L1 having the amino acid sequence of SEQ ID NO: 7 or 8 (CDR-L1 of 19B12 and 3C5, respectively) or a variant of SEQ ID NO: 7 or 8 modified by at most two highly conservative amino acid substitutions; a CDR-L2 having the amino acid sequence of SEQ ID NO: 9 (CDR-L2 of 19B12 and 3C5) or a variant thereof modified by one highly conservative amino acid substitution; and a CDR-L3 having the amino acid sequence of SEQ ID NO: 10 (CDR-L3 of 19B12 and 3C5) or a variant thereof modified by one highly conservative amino acid substitution.

In specific embodiments, the monoclonal antibody or antigen binding fragment comprises
(i) a heavy chain variable domain (VH) comprising a CDR-H1 consisting of the amino acid sequence of SEQ ID NO: 3 or a variant thereof modified by one highly conservative amino acid substitution; a CDR-H2 consisting of the amino acid sequence of SEQ ID NO: 4 or 5 or a variant of SEQ ID NO: 4 or 5 modified by at most two highly conservative amino acid substitutions; and a CDR-H3 consisting of the amino acid sequence of SEQ ID NO: 6 or a variant thereof modified by one highly conservative amino acid substitution; and
(ii) a light chain variable domain (VL) comprising a CDR-L1 consisting of the amino acid sequence of SEQ ID NO: 7 or 8 or a variant of SEQ ID NO: 7 or 8 modified by at most two highly conservative amino acid substitutions; a CDR-L2 consisting of the amino acid sequence of SEQ ID NO: 9 or a variant thereof modified by one highly conservative amino acid substitution; and a CDR-L3 consisting of the amino acid sequence of SEQ ID NO: 10 or a variant thereof modified by one highly conservative amino acid substitution.

The amino acid sequences in the CDRs of the 1,6fucAFP antibodies 19B12 and 3C5 differ in three amino acids. Specifically, there is an insertion of an amino acid and an amino acid substitution in the CDR-H2 of 19B12 (i.e. SEQ ID NO: 4) relative to the CDR-H2 of 3C5 (i.e. SEQ ID NO: 5). Further, the CDR-L1 of 19B12 (i.e. SEQ ID NO: 7) and the CDR-L1 of 3C5 (i.e. SEQ ID NO: 8) differ in one amino acid.

Accordingly, the 1,6fucAFP antibody or antigen binding fragment provided herein may comprise
(i) a heavy chain variable domain (VH) comprising a CDR-H1 having the amino acid sequence of SEQ ID NO: 3; a CDR-H2 having the amino acid sequence of SEQ ID NO: 4 or 5 or a variant of SEQ ID NO: 4 or 5 modified by one amino acid substitution; and a CDR-H3 having the amino acid sequence of SEQ ID NO: 6; and
(ii) a light chain variable domain (VL) comprising a CDR-L1 having the amino acid sequence of SEQ ID NO: 7 or 8 or a variant of SEQ ID NO: 7 or 8 modified by one amino acid substitution; a CDR-L2 having the amino acid sequence of SEQ ID NO: 9; and a CDR-L3 having the amino acid sequence of SEQ ID NO: 10.

In specific embodiments, the monoclonal antibody or antigen binding fragment may comprise
(i) a heavy chain variable domain (VH) comprising a CDR-H1 consisting of the amino acid sequence of SEQ ID NO: 3; a CDR-H2 consisting of the amino acid sequence of SEQ ID NO: 4 or 5 or a variant of SEQ ID NO: 4 or 5 modified by one amino acid substitution; and a CDR-H3 consisting of the amino acid sequence of SEQ ID NO: 6; and
(ii) a light chain variable domain (VL) comprising a CDR-L1 consisting of the amino acid sequence of SEQ ID NO: 7 or 8 or a variant of SEQ ID NO: 7 or 8 modified by one amino acid substitution; a CDR-L2 consisting of the amino acid sequence of SEQ ID NO: 9; and a CDR-L3 consisting of the amino acid sequence of SEQ ID NO: 10.

In embodiments the antibody or antigen binding fragment comprises
(i) a heavy chain variable domain (VH) comprising a CDR-H1 having the amino acid sequence of SEQ ID NO: 3; a CDR-H2 having the amino acid sequence of SEQ ID NO: 4 or 5 or a variant of SEQ ID NO: 4 or 5 modified by one conservative amino acid substitution; and a CDR-H3 having the amino acid sequence of SEQ ID NO: 6; and
(ii) a light chain variable domain (VL) comprising a CDR-L1 having the amino acid sequence of SEQ ID NO: 7 or 8 or a variant of SEQ ID NO: 7 or 8 modified by one conservative amino acid substitution; a CDR-L2 having the amino acid sequence of SEQ ID NO: 9; and a CDR-L3 having the amino acid sequence of SEQ ID NO: 10.

In specific embodiments, the monoclonal antibody or antigen binding fragment may comprise
(i) a heavy chain variable domain (VH) comprising a CDR-H1 consisting of the amino acid sequence of SEQ ID NO: 3; a CDR-H2 consisting of the amino acid sequence of SEQ ID NO: 4 or 5 or a variant of SEQ ID NO: 4 or 5 modified by one conservative amino acid substitution; and a CDR-H3 consisting of the amino acid sequence of SEQ ID NO: 6; and
(ii) a light chain variable domain (VL) comprising a CDR-L1 consisting of the amino acid sequence of SEQ ID NO: 7 or 8 or a variant of SEQ ID NO: 7 or 8 modified by one conservative amino acid substitution; a CDR-L2 consisting of the amino acid sequence of SEQ ID NO: 9; and a CDR-L3 consisting of the amino acid sequence of SEQ ID NO: 10.

In embodiments the antibody or antigen binding fragment comprises
(i) a heavy chain variable domain (VH) comprising a CDR-H1 having the amino acid sequence of SEQ ID NO: 3; a CDR-H2 having the amino acid sequence of SEQ ID NO: 4 or 5 or a variant of SEQ ID NO: 4 or 5 modified by one conservative or highly conservative amino acid substitution; and a CDR-H3 having the amino acid sequence of SEQ ID NO: 6; and
(ii) a light chain variable domain (VL) comprising a CDR-L1 having the amino acid sequence of SEQ ID NO: 7 or 8 or a variant of SEQ ID NO: 7 or 8 modified by one conservative or highly conservative amino acid substitution; a CDR-L2 having the amino acid sequence of SEQ ID NO: 9; and a CDR-L3 having the amino acid sequence of SEQ ID NO: 10.

In specific embodiments, the monoclonal antibody or antigen binding fragment may comprise
(i) a heavy chain variable domain (VH) comprising a CDR-H1 consisting of the amino acid sequence of SEQ ID NO: 3; a CDR-H2 consisting of the amino acid sequence of SEQ ID NO: 4 or 5 or a variant of SEQ ID NO: 4 or 5 modified by one conservative highly conservative amino acid substitution; and a CDR-H3 consisting of the amino acid sequence of SEQ ID NO: 6; and
(ii) a light chain variable domain (VL) comprising a CDR-L1 consisting of the amino acid sequence of SEQ ID NO: 7 or 8 or a variant of SEQ ID NO: 7 or 8 modified by one conservative highly conservative amino acid substitution; a CDR-L2 consisting of the amino acid sequence of SEQ ID NO: 9; and a CDR-L3 consisting of the amino acid sequence of SEQ ID NO: 10.

The amino acid sequences in the CDRs of the 1,6fucAFP antibodies 19B12 and 3C5 differ in three amino acids. Specifically, there is an insertion of an amino acid in position 3 of the CDR-H2 of 19B12 (i.e. SEQ ID NO: 4) relative to the CDR-H2 of 3C5 (i.e. SEQ ID NO: 5). Further, there is a difference in the amino acids in position 5 of CDR-H2 of 19B12 (i.e. SEQ ID NO: 4) and position 4 of the CDR-H2 of 3C5 (i.e. SEQ ID NO: 5). These position correspond to each other taking into account the aforementioned insertion. Finally, the CDR-L1 of 19B12 (i.e. SEQ ID NO: 7) and the CDR-L1 of 3C5 (i.e. SEQ ID NO: 8) differ in the amino acid at position 7.

Thus, the monoclonal 1,6fucAFP antibody or antigen binding fragment of the present disclosure may comprise
(i) a heavy chain variable domain (VH) comprising a CDR-H1 having the amino acid sequence of SEQ ID NO: 3; a CDR-H2 having the amino acid sequence of SEQ ID NO: 4 or 5 or a variant of SEQ ID NO: 4 modified by one amino acid substitution at amino acid position 5 or a variant of SEQ ID NO:5 modified by one amino acid substitution at amino acid position 4; and a CDR-H3 having the amino acid sequence of SEQ ID NO: 6; and
(ii) a light chain variable domain (VL) comprising a CDR-L1 having the amino acid sequence of SEQ ID NO: 7 or 8 or a variant of SEQ ID NO: 7 or 8 modified by one amino acid substitution at amino acid position 7; a CDR-L2 having the amino acid sequence of SEQ ID NO: 9; and a CDR-L3 having the amino acid sequence of SEQ ID NO: 10.

In a more specific embodiment the monoclonal antibody or antigen binding fragment of the present disclosure comprises
(i) a heavy chain variable domain (VH) comprising a CDR-H1 consisting of the amino acid sequence of SEQ ID NO: 3; a CDR-H2 consisting of the amino acid sequence of SEQ ID NO: 4 or 5 or a variant of SEQ ID NO: 4 modified by one amino acid substitution at amino acid position 5 or a variant of SEQ ID NO:5 modified by one amino acid substitution at amino acid position 4; and a CDR-H3 consisting of the amino acid sequence of SEQ ID NO: 6; and
(ii) a light chain variable domain (VL) comprising a CDR-L1 consisting of the amino acid sequence of SEQ ID NO: 7 or 8 or a variant of SEQ ID NO: 7 or 8 modified by one amino acid substitution at amino acid position 7; a CDR-L2 consisting of the amino acid sequence of SEQ ID NO: 9; and a CDR-L3 consisting of the amino acid sequence of SEQ ID NO: 10.

In embodiments, the monoclonal antibody or antigen binding fragment of the present disclosure comprises
(i) a heavy chain variable domain (VH) comprising a CDR-H1 having the amino acid sequence of SEQ ID NO: 3; a CDR-H2 having the amino acid sequence of SEQ ID NO: 4 or 5 or a variant of SEQ ID NO: 4 modified by one conservative amino acid substitution at amino acid position 5 or a variant of SEQ ID NO:5 modified by one conservative amino acid substitution at amino acid position 4; and a CDR-H3 having the amino acid sequence of SEQ ID NO: 6; and
(ii) a light chain variable domain (VL) comprising a CDR-L1 having the amino acid sequence of SEQ ID NO: 7 or 8 or a variant of SEQ ID NO: 7 or 8 modified by one conservative amino acid substitution at amino acid position 7; a CDR-L2 having the amino acid sequence of SEQ ID NO: 9; and a CDR-L3 having the amino acid sequence of SEQ ID NO: 10.

In a more specific embodiment the monoclonal antibody or antigen binding fragment of the present disclosure comprises
(i) a heavy chain variable domain (VH) comprising a CDR-H1 consisting of the amino acid sequence of SEQ ID NO: 3; a CDR-H2 consisting of the amino acid sequence of SEQ ID NO: 4 or 5 or a variant of SEQ ID NO: 4 modified by one conservative amino acid substitution at amino acid position 5 or a variant of SEQ ID NO:5 modified by one conservative amino acid substitution at amino acid position 4; and a CDR-H3 consisting of the amino acid sequence of SEQ ID NO: 6; and
(ii) a light chain variable domain (VL) comprising a CDR-L1 consisting of the amino acid sequence of SEQ ID NO: 7 or 8 or a variant of SEQ ID NO: 7 or 8 modified by one conservative amino acid substitution at amino acid position 7; a CDR-L2 consisting of the amino acid sequence of SEQ ID NO: 9; and a CDR-L3 consisting of the amino acid sequence of SEQ ID NO: 10.

In embodiments, the monoclonal antibody or antigen binding fragment of the present disclosure comprises
(i) a heavy chain variable domain (VH) comprising a CDR-H1 having the amino acid sequence of SEQ ID NO: 3; a CDR-H2 having the amino acid sequence of SEQ ID NO: 4 or 5 or a variant of SEQ ID NO: 4 modified by one highly conservative amino acid substitution at amino acid position 5 or a variant of SEQ ID NO:5 modified by one highly conservative amino acid substitution at amino acid position 4; and a CDR-H3 having the amino acid sequence of SEQ ID NO: 6; and
(ii) a light chain variable domain (VL) comprising a CDR-L1 having the amino acid sequence of SEQ ID NO: 7 or 8 or a variant of SEQ ID NO: 7 or 8 modified by one highly conservative amino acid substitution at amino acid position 7; a CDR-L2 having the amino acid sequence of SEQ ID NO: 9; and a CDR-L3 having the amino acid sequence of SEQ ID NO: 10.

In a more specific embodiment the monoclonal antibody or antigen binding fragment of the present disclosure comprises
(i) a heavy chain variable domain (VH) comprising a CDR-H1 consisting of the amino acid sequence of SEQ ID NO: 3; a CDR-H2 consisting of the amino acid sequence of SEQ ID NO: 4 or 5 or a variant of SEQ ID NO: 4 modified by one highly conservative amino acid substitution at amino acid position 5 or a variant of SEQ ID NO:5 modified by one highly conservative amino acid substitution at amino acid position 4; and a CDR-H3 consisting of the amino acid sequence of SEQ ID NO: 6; and
(ii) a light chain variable domain (VL) comprising a CDR-L1 consisting of the amino acid sequence of SEQ ID NO: 7 or 8 or a variant of SEQ ID NO: 7 or 8 modified by one highly conservative amino acid substitution at amino acid position 7; a CDR-L2 consisting of the amino acid sequence of SEQ ID NO: 9; and a CDR-L3 consisting of the amino acid sequence of SEQ ID NO: 10.

If not specified otherwise, the amino acid substitution(s) in any one of (e.g. in all) the variants of SEQ ID NOs: 3 to 10 may be any amino acid exchange. In specific embodiments, the amino acid substitution(s) in any one of (e.g. in all) the variants SEQ ID NOs: 3 to 10 may be conservative amino acid exchange(s). In even more specific embodiments, the amino acid substitution(s) in any one of (e.g. in all) the variants SEQ ID NOs: 3 to 10 may be highly conservative amino acid exchange(s).

In embodiments, the monoclonal antibody or antigen binding fragment of the present disclosure comprises
(i) a heavy chain variable domain (VH) comprising a CDR-H1 having the amino acid sequence of SEQ ID NO: 3; a CDR-H2 having the amino acid sequence of SEQ ID NO: 4 or 5 or a variant of SEQ ID NO: 4 modified by one amino acid substitution at amino acid position 5 or a variant of SEQ ID NO:5 modified by one amino acid substitution at amino acid position 4; and a CDR-H3 having the amino acid sequence of SEQ ID NO: 6; and
(ii) a light chain variable domain (VL) comprising a CDR-L1 having the amino acid sequence of SEQ ID NO: 7 or 8; a CDR-L2 having the amino acid sequence of SEQ ID NO: 9; and a CDR-L3 having the amino acid sequence of SEQ ID NO: 10, wherein the amino acid position 5 in the variant of SEQ ID NO: 4 is modified to a serine, and wherein the amino acid position 4 in the variant of SEQ ID NO:5 is modified to an asparagine.

In a more specific embodiment the monoclonal antibody or antigen binding fragment of the present disclosure comprises
(i) a heavy chain variable domain (VH) comprising a CDR-H1 consisting of the amino acid sequence of SEQ ID NO: 3; a CDR-H2 consisting of the amino acid sequence of SEQ ID NO: 4 or 5 or a variant of SEQ ID NO: 4 modified by one amino acid substitution at amino acid position 5 or a variant of SEQ ID NO:5 modified by one amino acid substitution at amino acid position 4; and a CDR-H3 consisting of the amino acid sequence of SEQ ID NO: 6; and
(ii) a light chain variable domain (VL) comprising a CDR-L1 consisting of the amino acid sequence of SEQ ID NO: 7 or 8; a CDR-L2 consisting of the amino acid sequence of SEQ ID NO: 9; and a CDR-L3 consisting of the amino acid sequence of SEQ ID NO: 10;
   wherein the amino acid position 5 in the variant of SEQ ID NO: 4 is modified to a serine, and wherein the amino acid position 4 in the variant of SEQ ID NO:5 is modified to an asparagine.

The monoclonal antibody or the antigen binding fragment according to the invention may comprise the CDR sequences of the monoclonal antibody 19B12 as identified herein.

Accordingly, the monoclonal antibody or antigen binding fragment of the invention may comprise:
(i) a heavy chain variable domain (VH) comprising a CDR-H1 consisting of the amino acid sequence of SEQ ID NO: 3; a CDR-H2 consisting of the amino acid sequence of SEQ ID NO: 4; and a CDR-H3 consisting of the amino acid sequence of SEQ ID NO: 6; and
(ii) a light chain variable domain (VL) comprising a CDR-L1 consisting of the amino acid sequence of SEQ ID NO: 7; a CDR-L2 consisting of the amino acid sequence of SEQ ID NO: 9; and a CDR-L3 consisting of the amino acid sequence of SEQ ID NO: 10.

The monoclonal antibody or the antigen binding fragment according to the invention may comprise the CDR sequences of the monoclonal antibody 3C5 as identified herein.

Thus, the monoclonal antibody or antigen binding fragment of the present disclosure may comprises
(i) a heavy chain variable domain (VH) comprising a CDR-H1 consisting of the amino acid sequence of SEQ ID NO: 3; a CDR-H2 consisting of the amino acid sequence of SEQ ID NO: 5; and a CDR-H3 consisting of the amino acid sequence of SEQ ID NO: 6; and
(ii) a light chain variable domain (VL) comprising a CDR-L1 consisting of the amino acid sequence of SEQ ID NO: 8; a CDR-L2 consisting of the amino acid sequence of SEQ ID NO: 9; and a CDR-L3 consisting of the amino acid sequence of SEQ ID NO: 10.

Herein above a number of embodiments reciting the amino acid sequences as depicted in SEQ ID NOs: 3, 4, 5, 6, 7, 8, 9 and 10 and optionally variants thereof are disclosed. Provided herein are also corresponding embodiments in which the reference to “SEQ ID NO: 3” is replaced by “the CDR-H1 sequence as comprised in SEQ ID NO: 11 or 12”, the reference to “SEQ ID NO: 4” is replaced by “the CDR-H2 sequence as comprised in SEQ ID NO: 11”, the reference to “SEQ ID NO: 5” is replaced by “the CDR-H2 sequence as comprised in SEQ ID NO: 12”, the reference to “SEQ ID NO: 6” is replaced by “the CDR-H3 sequence as comprised in SEQ ID NO: 11 or 12, the reference to “SEQ ID NO: 7” is replaced by “the CDR-L1 sequence as comprised in SEQ ID NO: 11”, the reference to “SEQ ID NO: 8” is replaced by “the CDR-L1 sequence as comprised in SEQ ID NO: 12”, the reference to “SEQ ID NO: 9” is replaced by “the CDR-L2 sequence as comprised in SEQ ID NO: 11 or 12”, and/or the reference to “SEQ ID NO: 10” is replaced by “the CDR-L1 sequence as comprised in SEQ ID NO: 11 or 12”.

In embodiments, the monoclonal antibody or antigen binding fragment thereof comprise
(i) a heavy chain variable domain (VH) having an amino acid sequence with at least 80%, at least 85%, at least 90%, at least 95%, or preferably at least 97.5% sequence identity to SEQ ID NO:11 or 12; and
(ii) a light chain variable domain (VL) having an amino acid sequence with at least 80%, at least 85%, at least 90%, at least 95%, or preferably at least 97.3% sequence identity to SEQ ID NO: 13 or 14.

Accordingly, provided herein is a monoclonal antibody or antigen binding fragment thereof specifically binding to α-1,6-core-fucosylated alpha-fetoprotein (AFP) or a partial sequence of AFP comprising said α-1,6-core-fucosylation (e.g. the glycopeptide of formula I), which comprises
(i) a heavy chain variable domain (VH) having the amino acid sequence of SEQ ID NO:11 or 12 or an amino acid sequence with at least 80%, at least 85%, at least 90%, at least 95%, or preferably at least 97.5% sequence identity to SEQ ID NO:11 or 12; and
(ii) a light chain variable domain (VL) having the amino acid sequence of SEQ ID NO:13 or 14 or an amino acid sequence with at least 80%, at least 85%, at least 90%, at least 95%, or preferably at least 97.3% sequence identity to SEQ ID NO: 13 or 14.

As evident from the appended Examples and Figures the two antibodies 19B12 and 3C5 identified herein show a significant sequence similarity in the CDRs and also the framework regions of the VH and VL, but also have a number of amino acid substitutions. Based on the VH and VL sequences of these antibodies further antibodies with similar features (e.g. specificity towards α-1,6-core-fucosylated AFP, affinity and other kinetic parameters) can be generated that share sequence similarity with 19 B12 an 3C5. For instance, such variant antibodies could be generated by affinity maturation.

Accordingly, in embodiments the antibodies or antigen binding fragments of the invention may comprise:
(i) a heavy chain variable domain (VH) having the amino acid sequence of SEQ ID NO:11 or 12; or an affinity maturated variant of SEQ ID NO: 11 or 12; and
(ii) a light chain variable domain (VL) having the amino acid sequence of SEQ ID NO: 13 or 14 or an affinity maturated variant of SEQ ID NO:13 or 14.

In embodiments, the antibodies or antigen binding fragments of the invention may comprise
(i) a heavy chain variable domain (VH) comprising a CDR-H1 consisting of the amino acid sequence of SEQ ID NO: 3; a CDR-H2 consisting of the amino acid sequence of SEQ ID NO: 4 or 5 or a variant of SEQ ID NO: 4 modified by one amino acid substitution at amino acid position 5 or a variant of SEQ ID NO:5 modified by one amino acid substitution at amino acid position 4; and a CDR-H3 consisting of the amino acid sequence of SEQ ID NO: 6; or an affinity maturated variant of said VH; and
(ii) a light chain variable domain (VL) comprising a CDR-L1 consisting of the amino acid sequence of SEQ ID NO: 7 or 8; a CDR-L2 consisting of the amino acid sequence of SEQ ID NO: 9; and a CDR-L3 consisting of the amino acid sequence of SEQ ID NO: 10; or an affinity maturated variant of said VL.

Affinity maturated variants have by definition at least the same affinity as 19B12 or 3C5 and show at least the same binding preferences (i.e. discrimination in binding of distinct structures) as 19B12 or 3C5. Affinity maturated variants may comprise amino acid substitutions, insertions and/or deletions. In embodiments, affinity maturated variants may comprise amino acid substitutions. In embodiments, the affinity maturated variants show a sequence identity of at least 80%, at least 85%, at least 90%, at least 95%, at least 98% or at least 99% with the respective parent VH or VL sequence (e.g. any one of SEQ ID NOs 11 to 14).

It is known in the art that the antibody heavy or light chain variable domain comprises in addition to three CDRs, 4 framework domains. Specifically, it is known that framework region 1 (FW1) represents the most N-terminal portion of the variable chain domain, and framework region 4 (FW4) represents the most C-terminal part, with the CDRs interspersed between the framework regions:
FW1-CDR1-FW2-CDR2-FW3-CDR3-FW4

Although it is evident from the context whether the framework region (FW) or complementary determining region (CDR) of the heavy or light chain variable domain is referenced, FWs and CDRs are distinguished herein with the indicator “H” or “L”. For example, the component FWs and CDRs of a heavy chain variable domain are referenced herein as schematically represented below:
(FW-H1)-(CDR-H1)-(FW-H2)-(CDR-H2)-(FW-H3)-(CDR-H3)-(FW-H4)

Similarly, the component FWs and CDRs of a light chain variable domain are referenced herein as schematically represented below:
(FW-L1)-(CDR-L1)-(FW-L2)-(CDR-L2)-(FW-L3)-(CDR-L3)-(FW-L4).

In embodiments, the VH of the α-1,6-core-fucosylated AFP specific antibody or antigen binding fragment of the invention comprises framework regions (FW) and has the structure of:
FW-H1-CDR-H1-FW-H2-CDR-H2-FW-H3-CDR-H3-FW-H4.

The FW-H1 may comprise or consist of the amino acid sequence of SEQ ID NO: 15 or a variant thereof having at least 60%, preferably at least 70%, even more preferably at least 80%, even more preferably at least 85%, even more preferably at least 90%, even more preferably at least 95%, even more preferably at least 98% or even more preferably at least 99% thereto.

The FW-H2 may comprise or consist of the amino acid sequence of SEQ ID NO: 16 or 17 or a variant of SEQ ID NO: 16 or 17 having at least 60%, preferably at least 70%, even more preferably at least 80%, even more preferably at least 85%, even more preferably at least 90%, even more preferably at least 95%, even more preferably at least 98% or even more preferably at least 99% thereto.

The FW-H3 may comprise or consist of the amino acid sequence of SEQ ID NO: 18 or a variant thereof having at least 60%, preferably at least 70%, even more preferably at least 80%, even more preferably at least 85%, even more preferably at least 90%, even more preferably at least 95%, even more preferably at least 98% or even more preferably at least 99% thereto.

The FW-H4 may comprise or consist of the amino acid sequence of SEQ ID NO: 19 or a variant thereof having at least 60%, preferably at least 70%, even more preferably at least 80%, even more preferably at least 85%, even more preferably at least 90%, even more preferably at least 95%, even more preferably at least 98% or even more preferably at least 99% thereto.

In embodiments, the framework sequences of the VH have at most 2 or at most one amino acid substitution, insertion and/or deletion events.

In embodiments, the VL of the α-1,6-core-fucosylated AFP specific antibody or antigen binding fragment of the invention comprises framework regions (FW) and has the structure of:
FW-L1-CDR-L1-FW-L2-CDR-L2-FW-L3-CDR-L3-FW-L4.

The FW-L1 may comprise or consist of the amino acid sequence of SEQ ID NO: 20 or a variant thereof having at least 60%, preferably at least 70%, even more preferably at least 80%, even more preferably at least 85%, even more preferably at least 90%, even more preferably at least 95%, even more preferably at least 98% or even more preferably at least 99% thereto.

The FW-L2 may comprise or consist of the amino acid sequence of SEQ ID NO: 21 or 22 or a variant of SEQ ID NO: 21 or 22 having at least 60%, preferably at least 70%, even more preferably at least 80%, even more preferably at least 85%, even more preferably at least 90%, even more preferably at least 95%, even more preferably at least 98%, or even more preferably at least 99% thereto.

The FW-L3 may comprise or consist of the amino acid sequence of SEQ ID NO: 23 or a variant thereof having at least 60%, preferably at least 70%, even more preferably at least 80%, even more preferably at least 85%, even more preferably at least 90%, even more preferably at least 95%, even more preferably at least 98% or even more preferably at least 99% thereto.

The FW-L4 may comprise or consist of the amino acid sequence of SEQ ID NO: 24 or a variant thereof having at least 60%, preferably at least 70%, even more preferably at least 80%, even more preferably at least 85%, even more preferably at least 90%, even more preferably at least 95%, even more preferably at least 98% or even more preferably at least 99% thereto.

In embodiments, the framework sequences of the VH have at most 4, at most 3, at most 2 or at most 1 amino acid substitution, insertion and/or deletion events.

In embodiments, the framework regions of the VH and/or VL of the antibodies or antigen binding fragments of the invention may comprise rabbit derived framework sequences FW1, FW2, FW3 and FW4.

In embodiments, the framework regions of the VH and or VL of the antibodies or antigen binding fragments of the invention are derived from the same (rabbit) germline as the framework regions of 19B12 or 3C5.

In embodiments, the monoclonal antibodies and antigen-binding fragments of the invention may be rabbit antibodies.

In embodiments, the monoclonal antibodies and antigen-binding fragments of the invention may have a detection label attached thereto.

In embodiments, the monoclonal antibodies and antigen-binding fragments of the invention may have a capture label attached thereto.

The present inventors have found that using a multivalent 1,6fucAFP antibodies comprising the Fv domain of 19B12 more than 2 times (as in a conventional antibody), e.g. 8-times (also referred to herein as p8), the signal to noise ratios of the immunoassays as described in the appended examples could be increased.

Accordingly, in embodiments, the 1,6fucAFP antibodies of the invention may be multivalent antibodies comprising at least 3, at least 4, at least 5, at least 6, at least 7 or at least 8 Fv domains formed by any of the combinations the VH and VL domains as described herein above in the context of the heavy and light chains of the antibodies of the invention. In embodiments, the 1,6fucAFP antibodies of the invention may comprise any of the combinations of VH and VL domains as described herein at least 3, at least 4, at least 5, at least 6, at least 7 or at least 8 times (e.g. 2, 3, 4, 5, 6, 7, 8, 9 or 10 times). In embodiments, the 1,6fucAFP antibodies of the invention may comprise at least 3, at least 4, at least 5, at least 6, at least 7 or at least 8 Fab domains comprising a Fv formed by any of the combinations of VH and VL domains as described herein. In an embodiment, a multivalent antibody of the invention comprises the Fv domain (formed by VH and VL) of the antibody 19B12 or variants thereof as defined herein above in at least 3, at least 4, at least 5, at least 6, at least 7, at least 8 or at least 10 copies (e.g. 2, 3, 4, 5, 6, 7, 8, 9 or 10 copies).

Multivalent antibody as used herein relates to antibodies comprising at least three Fv domains. In preferred embodiments, multivalent antibodies comprise the same Fv in at least 3, at least 4, at least 5, at least 6, at least 7, at least 8 or at least 10 copies (e.g. 2, 3, 4, 5, 6, 7, 8, 9 or 10 copies).

Exemplary but non-limiting embodiments for multivalent antibodies and methods for generating such antibodies are disclosed in WO2019/057816, which is herein incorporated by reference in its entirety. Specifically, all embodiments relating to structural configurations of such multivalent antibodies and the methods for generating such multivalent antibodies (also referred to as p3, p4, p5, p6, p7 or p8) are disclosed herein by reference.

In embodiments, a multivalent antibody of the invention may comprise a heavy chain comprising a plurality of (e.g. 2, 3, 4, 5, 6, 7, 8, 9 or 10 copies, in a particular embodiment 8) VH-CH1 domains (e.g. all comprising a (preferably the same) VH of an 1,6fucAFP antibody or antigen binding fragment of the invention), e.g. flanked by linker sequences (e.g. one of the linker sequences as described in WO2019/057816, which are incorporated herein by reference). The additional VH-CH1 domains compared to a conventional antibody may be placed upstream and/or downstream of the Hinge-CH2-CH3 sequences. The light chains in such multimeric antibodies may be conventional light chains consisting of a VL and a constant domain.

Alternative methods for generating multivalent antibodies involve the chemical polymerization/crosslinking of antibodies or antigen binding fragments.

Antibodies of the invention may be generated using immunization and selection methods as described in the appended Examples.

Another suitable method for producing or isolating antibodies and antibody antigen binding fragments of the invention include, but are not limited to, methods that select a recombinant antibody from a peptide or protein library (e.g., but not limited to, a bacteriophage, ribosome, oligonucleotide, RNA, cDNA, or yeast display library) using binding activities of interest. For example, antibodies or antigen binding fragments can be selected from such libraries by positively selecting for specific binding to the glycopeptide of Formula I and negatively selecting for binding to the glycopeptide of Formula III and/or the core-fucosylated glycan of Formula IV and/or the peptide of SEQ ID NO: 2 or 25. Display libraries are well known in the art and are, for example, available from various commercial vendors including but not limited to Cambridge Antibody Technologies (Cambridgeshire, UK), MorphoSys (Martinsried/Planegg, Del.), Biovation (Aberdeen, Scotland, UK) and Bioinvent (Lund, Sweden). Again, selected clones can be processed according to routine methods for subsequent recombinant processing.

Variants of the antibodies or antigen-binding fragments disclosed herein can be generated using routine recombinant DNA technology using the sequences disclosed herein. For example, recombinant DNA technology may be used to remove or modify the DNA sequences encoding the antibodies and/or antibody antigen binding fragments disclosed herein, e.g. encoding the heavy and/or light chain variable domains as defined herein above. For example, recombinant DNA technology may be used to substitute or remove parts of the encoding sequence(s) that are not necessary for maintaining specific and selective binding to the antigen(s) of interest. The molecules expressed from such modified or truncated DNA molecules are also encompassed by the antibodies of the invention.

Antibodies of the invention may be expressed recombinantly. Accordingly, in certain embodiments, the monoclonal antibody of the invention may be a recombinant antibody. Methods for producing a recombinant antibody are known in the art. Exemplary embodiments for antibody expression are disclosed in the appended Examples and herein elsewhere.

In a second aspect, the present invention provides a nucleic acid molecule or a set of polynucleotides encoding the 1,6fucAFP antibodies or 1,6fucAFP antigen binding fragments disclosed herein. In particular, provided is a polynucleotide encoding an 1,6fucAFP heavy chain and/or light chain variable domain as defined herein above. As used herein, “nucleic acid molecule”, “nucleic acid sequence”, “polynucleotide” and analogous terms include both genomic DNA and cDNA, as well as RNA capable of driving expression of an antibody or antigen binding fragment of the invention. It is understood that the term “RNA” as used herein comprises all forms of RNA including mRNA, tRNA and rRNA but also genomic RNA, such as in case of RNA of RNA viruses. Preferably, embodiments reciting “RNA” are directed to mRNA. The nucleic acid molecules/nucleic acid sequences of the invention may be of natural as well as of synthetic or semi-synthetic origin. Thus, the nucleic acid molecules may, for example, be nucleic acid molecules that have been synthesized according to conventional protocols of organic chemistry, according to recombinant methods, or produced semi-synthetically, e.g. by combining chemical synthesis and recombinant methods. The person skilled in the art is familiar with the preparation and the use of such nucleic acid molecules. A “set of polynucleotides” relates to at least two polynucleotides on which parts of the antibody or antigen binding fragment are encoded. For example, a first polynucleotide encoding the heavy chain variable domain and a second polynucleotide encoding the light chain variable domain may be used.

In embodiments, provided is a polynucleotide or a set of polynucleotides encoding one of the VH and VL combinations disclosed in the context of the first aspect of the invention. Accordingly, for each of the aspects and embodiments relating to 1,6fucAFP antibodies or antigen binding fragments thereof as described herein a corresponding polynucleotide encoding the respective antibody or antigen binding fragment is provided mutatis mutandis.

In particular embodiments, provided is a polynucleotide or a set of polynucleotides encoding the antibody heavy chain variable domain VH of SEQ ID NO: 11 or 12 or variants thereof as defined in the context of the first aspect of the invention and/or the antibody light chain variable domain of SEQ ID NO: 13 or 14 or variants thereof as defined in the context of the first aspect of the invention.

In another particular embodiment provided is a polynucleotide or a set of polynucleotides encoding the VH of SEQ ID NO: 11 and the VL of SEQ ID NO: 13.

In another particular embodiment provided is a polynucleotide or a set of polynucleotides encoding the VH of SEQ ID NO: 12 and the VL of SEQ ID NO: 14.

In some embodiments, the polynucleotide may comprise further sequences to ensure that not only the heavy and/or light chain variable domain are expressed, but also the remaining heavy and/or light chain constant regions such that a full-length antibody (e.g. IgG) is expressed comprising the heavy and light chain variable domains of the invention.

In a third aspect, provided herein is a vector that comprises a polynucleotide or a set of polynucleotides according to the second aspect of the invention. In particular, provided are vectors comprising a nucleic acid molecule encoding an antibody or antibody antigen binding fragment of the invention. As used herein, the term “vector” relates to a circular or linear nucleic acid molecule that can autonomously replicate in a host cell into which it has been introduced. Non-limiting examples of vectors suitable for use in the present invention include cosmids, plasmids (e.g., naked or contained in liposomes), viruses (e.g., lentiviruses, retroviruses, adenoviruses, and adeno-associated viruses) and bacteriophages. However, the art provides many suitable vectors, the choice of which depends on the desired function. The development and use of suitable vectors is well documented in the art; see, for example, the techniques described in Sambrook and Russel “Molecular Cloning, A Laboratory Manual”, Cold Spring Harbor Laboratory, N.Y. (2001) and Ausubel, “Current Protocols in Molecular Biology”, Green Publishing Associates and Wiley Interscience, N.Y. (1989), (1994). Vectors of use in connection with the present invention comprise a nucleic acid sequence encoding an 1,6fucAFP antibody or an antigen binding fragment as disclosed herein. As such, for each of the aspects and embodiments relating to monoclonal antibodies or antigen binding fragments specifically binding to α-1,6-core-fucosylated AFP as described herein a vector comprising the corresponding polynucleotide encoding the respective antibody or antigen binding fragment is provided herein.

With regard to the term “vector comprising” as used herein, it is understood in the art that further nucleic acid sequences are present in the vectors that are necessary and/or sufficient for desired vector activity in the host cell, e.g. drive replication of the vector (and, thus the encoding nucleic acid sequences) and/or to direct the host cell express the antibody or antigen binding fragment of the invention. Such further nucleic acid sequences include but are not limited to sequences controlling vector replication and/or expression of a desired sequence in the particular cell system. For example, the vectors may comprise the nucleic acid molecule encoding an antibody or antibody antigen binding fragment of the invention operably linked and/or under the control of regulatory sequences. The term “regulatory sequence” refers to DNA sequences that are necessary to effect the expression of coding sequences to which they are operably linked. The term “control sequence” is intended to include, at a minimum, all components the presence of which may also be necessary for expression, and may further include additional advantageous components, e.g., to allow replication. As is understood in the art, the nature of such regulatory and control sequences differs depending upon the host organism. For example, in prokaryotes, control sequences generally include promoters, ribosomal binding sites, and terminators. In eukaryotes control sequences generally include promoters, terminators and, in some instances, enhancers, trans-activators and/or transcription factors.

The vectors of use in the present invention are preferably expression vectors. An expression vector is capable of directing the replication and the expression of the nucleic acid molecule of the invention in a host cell and, accordingly, provides for the expression of, e.g., the heavy chain and/or light chain variable domains of the 1,6fucAFP monoclonal antibodies or antigen binding fragments of the invention. In some embodiments, the vector may comprise further sequences to ensure that not only the heavy and light chain variable domains are expressed, but also the remaining heavy and light chain constant regions such that a full-length antibody (e.g. IgG) is expressed comprising the heavy and light chain variable domains of the invention. Suitable expression vectors have been widely described in the literature and the determination of the appropriate expression vector for a particular cell system can be readily made by the skilled person using routine methods. Preferably, the vectors disclosed herein comprise a recombinant polynucleotide (i.e., a nucleic acid sequence encoding the monoclonal antibody according to the invention) as well as expression operably linked control sequences. The vectors as provided herein preferably further comprise a promoter. The herein described vectors may also comprise a selection marker gene and a replication-origin ensuring replication in the host Moreover, the herein provided vectors may also comprise a termination signal for transcription. Expression vectors as known in the art may drive transient or constitutive expression in a host cell.

The nucleic acid molecules and/or vectors of the invention can be designed for transfection into prokaryotic or eukaryotic host cells by any means known in the art or described herein. Non-limiting examples of suitable methods include chemical based methods (polyethylenimine, calcium phosphate, liposomes, DEAE-dextrane, nucleofection), nonchemical methods (electroporation, sonoporation, optical transfection, gene electrotransfer, hydrodynamic delivery or naturally occurring transformation upon contacting cells with the nucleic acid molecule of the invention), particle-based methods (gene gun, magnetofection, impalefection) phage vector-based methods and viral methods. For example, expression vectors derived from viruses such as retroviruses, vaccinia virus, adeno-associated virus, herpes viruses, Semliki Forest Virus or bovine papilloma virus, may be used for transfection of the nucleic acid molecules into targeted cell population. Additionally, baculoviral systems can also be used as vector in eukaryotic expression system for the nucleic acid molecules of the invention.

The term “prokaryote” is meant to include all bacteria which can be transformed, transduced or transfected with DNA or DNA or RNA molecules for the expression of a protein of the invention. Prokaryotic hosts may include gram negative as well as gram positive bacteria such as, for example, E. coli, S. typhimurium, Serratia marcescens, Corynebacterium (glutamicum), Pseudomonas (fluorescens), Lactobacillus, Streptomyces, Salmonella and Bacillus subtilis. The term “eukaryotic” is meant to include yeast, higher plant, insect and mammalian cells. Non-limiting examples of mammalian host cells typically used in the art include, Hela, HEK293, H9, Per.C6 and Jurkat cells, mouse NIH3T3, NS/0, SP2/0 and C127 cells, COS cells, e.g. COS 1 or COS 7, CV1, quail QC1-3 cells, mouse L cells, mouse sarcoma cells, Bowes melanoma cells and Chinese hamster ovary (CHO) cells.

According to a fourth aspect, the present invention relates to a host cell comprises a polynucleotide or a set of polynucleotides according to the second aspect of the invention, or a vector according to the third aspect. The host cell may be configured such that an 1,6fucAFP antibody or antigen binding fragment provided herein is expressed. The host cell may be a prokaryotic cell or a eukaryotic cell. In a preferred embodiment, the host cell is a eukaryotic cell. Exemplary prokaryotic and eukaryotic cells are disclosed herein above in the context of the third aspect of the invention. In a particular embodiment, the cell is a HEK cell. In another particular embodiment, the host cell is a CHO cell.

It is preferred that the host cells in accordance with the present invention are eukaryotic cells (e.g. HEK or CHO). Although it is possible to express the antibodies and antigen binding fragments as disclosed herein in both prokaryotic and eukaryotic host cells, expression of antibodies in eukaryotic cells is preferred, and in mammalian host cells as most preferred, because such eukaryotic cells (in particular, the most preferred mammalian cells) are more likely to express a properly folded antibody/antibody fragment, containing the proper post-translational modifications such that it is immunologically active.

When recombinant expression vectors encoding the antibody heavy chain and/or light chains variable domain as disclosed herein are introduced into host cells, the antibodies or antibody antigen binding fragments are produced by culturing the host cells for a period of time sufficient to allow for expression of the antibody or antigen binding fragment in the host cell or, preferably, to allow for secretion of the antibody or antigen binding fragment into the culture medium in which the host cells are grown. Antibodies and/or antigen binding fragments can be recovered from the culture medium using standard protein purification methods.

Accordingly, in a fifth aspect, the invention also provides a method for the production of an 1,6fucAFP antibody or an 1,6fucAFP antibody antigen binding fragment as disclosed herein comprising culturing a host cell of the invention expressing an antibody or antigen binding fragment of the invention under suitable conditions and isolating the 1,6-fucAFP antibody or antigen binding fragment produced.

The transformed host cells can be grown in bioreactors and cultured according to techniques known in the art to achieve optimal cell growth. The antibody and/or antibody antigen binding fragment of the invention can then be isolated from the cell fraction or growth medium by any conventional means such, but not limited to, affinity chromatography (for example using a fusion-tag such as the Strep-tag II or the His₆ tag), gel filtration (size exclusion chromatography), anion exchange chromatography, cation exchange chromatography, hydrophobic interaction chromatography, high pressure liquid chromatography (HPLC), reversed phase HPLC or immunoprecipitation.

In sixth aspect, provided herein are 1,6-fucAFP antibodies or an antigen binding fragments obtainable or obtained by a method according to the fifth aspect of the invention.

In a seventh aspect, the present invention relates to a composition comprising an 1,6-fucAFP antibody or antigen binding fragment of the invention, a polynucleotide of the invention, a vector of the invention or a host cell of the invention. In a preferred embodiment, the composition is a diagnostic composition, i.e. a composition for use in diagnostic applications. In preferred embodiments, the composition is for use in an in vitro diagnostic test for detecting α-1,6-core-fucosylated AFP (i.e. the clinically relevant portion of AFP-L3). In a preferred embodiment the diagnostic composition may be a reagent for an immunoassay for detecting α-1,6-core-fucosylated AFP (i.e. the clinically relevant portion of AFP-L3). The diagnostic composition is preferably configured such that it allows for detection of α-1,6-core-fucosylated AFP (i.e. the clinically relevant portion of AFP-L3) in a sample obtained from a subject. The sample may be a body fluid. In particular embodiment, the sample may be a blood sample (e.g. whole blood, serum or plasma).

In embodiments, the composition of the invention is a composition for in vitro detection (preferably quantification) of α-1,6-core-fucosylated AFP (i.e. the clinically relevant portion of AFP-L3) in a sample, preferably using an immunoassay. In embodiments, the immunoassay is a heterogeneous immunoassay. In embodiments, the immunoassay is a sandwich immunoassay. In embodiments, the immunoassay is a competitive immunoassay. In embodiments, the immunoassay is an immunoassay of the invention.

The 1,6fucAFP antibodies and antibody antigen bind domains of the invention, and their methods of production and use, are provided not only as diagnostic tools but are also envisioned to have applicability in the treatment and amelioration of disease and disease symptoms, as well as applicability in model systems for investigating disease therapies. Accordingly, the invention provides pharmaceutical compositions comprising one or more pharmaceutically acceptable carriers and (i) an 1,6fucAFP antibody or an antigen binding fragment thereof, (ii) a polynucleotide encoding an antibody or antigen binding fragment of (i); (iii) a vector comprising a polynucleotide of (ii); or (iv) a host cell comprising a polynucleotide of (ii) and/or a vector of (iii) that expresses an antibody or antigen binding fragment of (i).

The pharmaceutical composition disclosed herein are formulated to be suitable for administration to a human or animal subject. In the manufacture of a pharmaceutical formulation, the antibodies or antigen binding fragments of the invention are admixed with a pharmaceutically acceptable carrier, excipient, and/or diluents. The carrier, excipient and/or diluent must, of course, be acceptable in the sense of being compatible with any other ingredients in the formulation and must not be deleterious to the subject. Examples of suitable pharmaceutical carriers for use with antibody-based compositions are well known in the art and can be formulated by conventional methods.

In an eighth aspect, herein provided is the use of an 1,6fucAFP antibody or antigen binding fragment according to the invention, or a composition according to the invention for an in vitro immunoassay, in particular an in vitro immunoassay for detecting (and optionally quantifying) α-1,6-core-fucosylated AFP (or a partial sequence thereof comprising α-1,6-core-fucosylated AFP) in a sample. As α-1,6-core-fucosylated AFP is the core component of AFP-L3, the immunoassay may also be referred to as an immunoassay for detecting (and optionally quantifying) AFP-L3 in a sample.

The sample may be a tissue slide or a body fluid, such as, but not restricted to, a blood sample, cerebrospinal fluid, seminal fluid, saliva or urine. In embodiments, the sample is a blood sample, such as whole blood, serum or plasma. In embodiments, the sample is serum or plasma.

Non-limiting examples of immunoassays are: enzyme linked immunosorbent assay (ELISA), enzyme immunoassay (EIA), radioimmunoassay (RIA), the Western blot assay, or immunoassays based on detection of luminescence, fluorescence, chemiluminescence or electrochemiluminescence.

Further exemplary immunoassay formats using 1,6-fucAFP antibody or antigen binding fragments (different antibodies then provided herein) for detection and/or quantification are also described in EP 3 252 073, US2018/0110889 and Egashira Y et al. (Scientific Reports, 2019, 9:12359), all of which are incorporated herein by reference in their entirety.

The principle of an immunoassay using an 1,6-fucAFP antibody or antigen binding fragment (or a composition comprising the same) according to the invention, is that the 1,6 fucAFP antibody or antigen binding fragment (or a composition comprising the same) is incubated with a sample (suspected to comprise or comprising α-1,6-core-fucosylated AFP). If the analyte of interest (i.e. α-1,6-core-fucosylated AFP) is present, a detection complex comprising the 1,6-fucAFP antibody or antigen binding fragment and α-1,6-core-fucosylated AFP is formed by an antibody antigen binding reaction/binding. To detect and/or quantify α-1,6-core-fucosylated AFP, the formation of the detection complex may be detected. Methods for detecting the formation of a detector complex are well known. For example, the detection may involve a detection label (e.g. attached to the antibody or antigen binding fragment of the invention).

Accordingly, the use according to the eighth aspect of the invention may comprise
a) incubating a sample comprising α-1,6-core-fucosylated AFP with a 1,6-fucAFP antibody or antigen binding fragment of the invention such that a detection complex comprising α-1,6-core-fucosylated AFP and the 1,6-fucAFP antibody or antigen binding fragment of the invention is formed; and
b) detecting the presence or the level of α-1,6-core-fucosylated AFP in the sample by detecting the detection complex.

As illustrated in the appended Examples, the signal in an immunoassay could be massively increased by using a pretreatment (e.g. a reducing pretreatment) of the sample comprising α-1,6-core-fucosylated AFP. The pretreatment seems to facilitate the accessibility of the epitope of the antibodies or antigen binding fragments of the invention.

Accordingly, the use according to the eighth aspect may comprise adding a pretreatment agent according to the invention (as described herein elsewhere) to the sample, preferably prior to the incubation of the sample with the 1,6-fucAFP antibody or antigen binding fragment of the invention.

The use may also comprise a further dilution of the sample after pretreatment, e.g. by adding a certain volume, preferably prior to or simultaneously with the addition of the 1,6-fucAFP antibody or antigen binding fragment of the invention to the sample. In other words, the concentration of the pretreatment agent may be reduced to a concentration acceptable for the formation of a detection complex. For instance, the concentration of the component of the pretreatment agent may be diluted between 2 and 3-fold.

In embodiments, the pretreatment may be a reducing pretreatment, i.e. may comprise the addition of a reduction agent (e.g. a protein reducing agent) to the sample. In other words, the pretreatment reagent may comprise or consist of a reducing agent.

A protein reducing agent is a reducing agent that can reduce/break down disulfide bonds within a protein/polypeptide chains or between proteins/polypeptide chains.

Non-limiting examples for reducing agents or protein reducing agents to be used for pretreatment are dithiothreitol (DTT), tris(2-carboxyethyl)phosphine (TCEP), β-mercaptoethanol and dithiobutylamine (DTBA). In embodiments, the reducing agent or protein reducing agent may be selected from dithiothreitol (DTT), tris(2-carboxyethyl)phosphine (TCEP) and dithiobutylamine (DTBA). In embodiments, the reducing agent or protein reducing agent may be selected from dithiothreitol (DTT) and tris(2-carboxyethyl)phosphine (TCEP). In embodiments, the reducing agent or protein reducing agent may be DTT.

In embodiments, a protein reducing agent having a redox potential at a pH of 7 which is 50% to 150% of the redox potential of DTT at a pH of 7 is employed. The redox potential of DTT at pH 7 is −0.33V.

The pH during the pretreatment is selected such that the reducing compound has efficient reducing power. As well known in the art, DTT has for example only efficient reducing activity at a pH 6.5 to 9.0 (preferably pH 7 to 8). TCEP is, for example, redox active at a pH range of about 1.5 to about 8.5.

The concentration of the (protein) reducing agent can be varied and depends on the precise reducing agent used. As illustrated in the appended Examples for DTT the concentrations to be used for an efficient pretreatment can be determined by comparing the immunoassay signal for a sample comprising a detectable level of α-1,6-core-fucosylated AFP using different concentrations of (protein) reducing agent prior to conducting a immunoassay that is otherwise identical.

Exemplary, if DTT is used as (protein) reducing agent, the final concentration of DTT in the sample during the pretreatment may be from 0.1 mM to 100 mM, 0.1 mM to 60 mM, 0.1 mM to 50 mM or 0.1 mM to 47.8 mM. In embodiments, the concentration of DTT may be at least 0.2 mM, at least 0.3 mM, at least 0.5 mM or at least 1 mM. In embodiments, the DTT concentration may be at most 100 mM, at most 60 mM, at most 50 mM or at most 47.8 mM. In embodiments, the concentration of DTT may be 2.2 mM. In embodiments, the concentration of DTT may be 2.3 mM. In embodiments, the DTT the final concentration of DTT in the mixture of pretreatment agent and sample may be from 0.6 mM to 107.8 mM. In embodiments, the DTT the final concentration of DTT in the mixture of pretreatment agent and sample may be from 0.7 mM to 107.8 mM.

The concentration of DTT in the pretreatment agent is higher than in the final mixture of the sample during the pretreatment. For instance, the pretreatment agent may be a 2-fold to 20-fold, or a 2-fold to 10-fold concentrate (e.g. a 2, 3, 4, 5, 6, 7, 8, 9, 10, 11, 12, 13, 14, 15, 16, 17, 18, 19 or 20 fold concentrate); i.e. stock solution.

The pretreatment agent used for pretreatment of the sample may further comprise a chelating agent, such as a chelating agent binding to ions. It has been found that addition of such chelating agent increases the stability of the pretreatment agent. In a particular embodiment, the chelating agent may be a chelating agent binding to ions. In a more specific embodiment, the chelating agent may be a chelating agent binding to divalent ions.

Non-limiting examples for chelating agents for binding to (divalent) ions are aminopolycarboxylic acids like ethylenediaminetetraacetic acid (EDTA) and diethylenetriaminepentaacetic acid (DTPA), or porphyrins, polyamines, crown ethers, ethylene glycol-bis(R-aminoethyl ether)-N,N,N′,N′-tetraacetic acid (EGTA).

Further non-limiting examples for chelating agents are DTPA, EC, DMSA, EDTA, EGTA, Cy-EDTA, EDTMP, DTPA, CyDTPA, Cy2DTPA, BOPTA, DTPA-MA, DTPA-BA, DTPMP, DOTA, TRITA, TETA, DOTMA, DOTA-MA, HP-DO3A, pNB-DOTA, DOTP, DOTMP, DOTEP, DOTPP, DOTBzP, DOTPME, HEDP, DTTP, anN3 S triamidethiol, DADS, MAMA, DADT, N2S4 diamine tetrathiol, N2P2 dithiol-. Bisphosphine, 6-hydrazinonicotinic acid, propyleneamine oxime, tetraamine, cyclam, or a combination thereof.

In embodiments, the chelating agent is EDTA.

The concentrations of the chelating agent during the pretreatment of the sample can be varied and may depend on the precise chelating agent and reducing agent used. Useful concentration ranges can be determined by comparing the immunoassay signal for a distinct sample comprising a detectable level of α-1,6-core-fucosylated AFP using different concentrations of chelating agent.

For example, if EDTA is used, exemplary concentrations during the pretreatment of the sample are 0.1 mM to 50 mM, 0.1 mM to 20 mM, 0.1 mM to 10 mM, 0.1 mM to 5 mM and 0.1 mM to 4 mM.

In embodiments, the pretreatment agent further comprises a buffer substance (also referred to as buffer herein). The buffer agent may be selected depending on the pH required for the protein reducing agent to reduce disulfide bonds.

In embodiments, the pretreatment agent comprises or consists of a protein reducing agent and a chelating agent for ions (e.g. divalent ions) and optionally a buffer.

In embodiments, the pretreatment agent comprises DTT and EDTA and optionally a buffer.

The pretreatment of the sample may be conducted for different times. For example, the pretreatment may be conducted from 1 min to 30 min, from 2 min to 20 min or 3 min to 10 min. In embodiments, the pretreatment may be conducted for 9 min.

In embodiments, the pretreatment may be an incubation of the sample with DTT at a pH 6.5 to 9.0 (preferably pH 7 to 8) for a predefined time (e.g. at least 2, 3, 4, 5, 6, 7, 8 or 9 min). Optionally also a chelating agent as described above may be present (e.g. EDTA). In embodiments, the pretreatment may also comprise addition of a buffer (e.g. Tris in concentrations of 10 mM to 200 mM, 30 mM to 150 mM, 50 mM to 130 mM, or 100 mM) to the sample adjusting the pH in the sample to above 7. After the pretreatment the concentration of DTT may be reduced such that the detection complex can still form.

In embodiments, the use may further comprise incubating the sample with a AFP-specific antibody that does not compete for binding to AFP with a 1,6-fucAFP antibody or antigen binding fragment of the invention. In these embodiments, the detection complex further comprises said AFP specific antibody, i.e. a sandwich comprising the 1,6-fucAFP antibody of the invention, the α-1,6-core-fucosylated AFP and the AFP-specific antibody that does not compete for binding to AFP with a 1,6-fucAFP antibody or antigen binding fragment of the invention. In the embodiments using a pretreatment, the AFP specific antibody shall be selected such that the antibody can still bind to AFP in the presence of the pretreatment agent.

A non-limiting example for an AFP-specific antibody that does not compete for binding with the antibodies of the invention is the commercially available anti-AFP antibody Tu11 (Roche Diagnostics Deutschland GmbH, material number: 11492080103). Accordingly, antibodies that do not compete for binding to AFP with the antibodies of the invention may be antibodies or antigen binding fragments thereof that have the same epitope as Tu11 or compete for binding to AFP with the Tu11 antibody. As demonstrated by the appended examples, the Tu11 antibody is also compatible with the pretreatment of the sample according to the present invention.

In embodiments, the use may comprise digesting the α-1,6-core-fucosylated AFP comprised in the sample to glycopeptides and peptides (e.g. using proteases) prior to the incubation with the antibody or antigen binding fragment of the invention, said glycopeptides comprising partial AFP sequences comprising α-1,6-core-fucosylated Asn-251 of AFP (e.g. as described herein elsewhere). Optionally the agents for generating glycopeptides or peptides may be removed or inactivated before adding the antibody or antigen binding fragment of the invention. The presence or level of α-1,6-core-fucosylated AFP in the sample may then be determined based on detection of a detection complex comprising the partial AFP sequences comprising α-1,6-core-fucosylated Asn-251 of AFP and the antibody or antigen binding fragment of the invention.

The immunoassay in which an 1,6fucAFP antibody or antigen binding fragment according to the invention is used may be an heterogeneous immunoassay.

Additionally or alternatively, an 1,6fucAFP antibody or antigen binding fragment according to the invention may be used in an sandwich immunoassay (e.g. a heterogeneous sandwich immunoassay).

In embodiments, the immunoassay may be an immunohistochemistry (IHC) assay.

In embodiments, the immunoassay may be for the detection of the glycopeptide of Formula I or a glycoprotein comprising the glycopeptide of Formula I.

As evident from the present disclosure the antibodies and antigen binding fragments of the invention can discriminate α-1,6-core-fucosylated AFP or a partial AFP sequence comprising said α-1,6-core-fucosylation from (i) AFP or a partial AFP sequence lacking the α-1,6-core-fucosylation and/or (ii) α-1,6-core-fucosylated proteins other than AFP. Thus, provided herein is also a use of the 1,6fucAFP antibodies and antigen binding fragments for discriminating α-1,6-core-fucosylated AFP or a partial AFP sequence comprising said α-1,6-core-fucosylation from (i) AFP or a partial AFP sequence lacking the α-1,6-core-fucosylation and/or (ii) α-1,6-core-fucosylated proteins other than AFP.

In a ninth aspect, the present invention provides an in vitro immunoassay method for detecting α-1,6-core-fucosylated AFP or a partial AFP sequence comprising said α-1,6-core-fucosylation in a sample using an 1,6-fucAFP antibody or antigen binding fragment of the invention.

The embodiments disclosed in the context of the use according to the eighth aspect apply mutatis mutandis.

In embodiments, the method according to the ninth aspect may comprise
(i) binding the antibody or antigen binding fragment of the invention to the α-1,6-core-fucosylated AFP or a partial AFP sequence comprising said α-1,6-core-fucosylation comprised in the sample so as to form a detection complex; and
(ii) detecting the α-1,6-core-fucosylated AFP or a partial AFP sequence comprising said α-1,6-core-fucosylation by detecting the detection complex and thereby determining the presence and optionally the amount of α-1,6-core-fucosylated AFP or a partial AFP sequence comprising said α-1,6-core-fucosylation in the sample.

In embodiments, the method according to the ninth aspect may comprise
a) incubating a sample comprising α-1,6-core-fucosylated AFP with a 1,6-fucAFP antibody or antigen binding fragment of the invention such that a detection complex comprising α-1,6-core-fucosylated AFP and the 1,6-fucAFP antibody or antigen binding fragment of the invention is formed; and
b) detecting the presence or the level of α-1,6-core-fucosylated AFP in the sample by detecting the detection complex.

As illustrated in the appended Examples, the signal in an immunoassay could be massively increased by using a pretreatment (e.g. a reducing pretreatment) of the sample comprising α-1,6-core-fucosylated AFP. The pretreatment seems to facilitate the accessibility of the epitope of the antibodies or antigen binding fragments of the invention in the context of a full-length α-1,6-core-fucosylated AFP.

Thus, the method according to the ninth aspect of the invention may comprise adding a pretreatment agent according to the invention to the sample, preferably prior to the incubation of the sample with the 1,6-fucAFP antibody or antigen binding fragment of the invention.

Accordingly, the method according to the ninth aspect may comprise
a) incubate the sample (e.g. a sample comprising α-1,6-core-fucosylated AFP) in the presence of a pretreatment reagent according to the invention;
b) incubating the pretreated sample with a 1,6-fucAFP antibody or antigen binding fragment of the invention such that a detection complex comprising α-1,6-core-fucosylated AFP and the 1,6-fucAFP antibody or antigen binding fragment of the invention is formed; and
c) detecting the presence or the level of α-1,6-core-fucosylated AFP in the sample by detecting the detection complex.

The method may comprise a further dilution of the sample after pretreatment, e.g. by adding a certain volume, preferably prior to or simultaneously with the addition of the antibody or antigen binding fragment of the invention to the sample. In other words, the concentration of the pretreatment agent may be reduced to a concentration acceptable for the formation of a detection complex.

In embodiments, the pretreatment may be a reducing pretreatment, i.e. may comprise the addition of a reduction agent (e.g. a protein reducing agent) to the sample. In other words, the pretreatment reagent may comprise or consist of a reducing agent (e.g. a protein reducing agent).

A protein reducing agent is a reducing agent that can reduce/break down disulfide bonds within a protein/polypeptide chains or between proteins/polypeptide chains.

Non-limiting examples for reducing agents or protein reducing agents to be used for pretreatment are dithiothreitol (DTT), tris(2-carboxyethyl)phosphine (TCEP), β-mercaptoethanol and dithiobutylamine (DTBA). In embodiments, the reducing agent or protein reducing agent may be selected from dithiothreitol (DTT), tris(2-carboxyethyl)phosphine (TCEP) and dithiobutylamine (DTBA). In embodiments, the reducing agent or protein reducing agent may be selected from dithiothreitol (DTT) and tris(2-carboxyethyl)phosphine (TCEP). In embodiments, the reducing agent or protein reducing agent may be dithiothreitol (DTT).

In embodiments, a protein reducing agent having a redox potential at a pH of 7 which is 50% to 150% of the redox potential of DTT at a pH of 7 is employed. The redox potential of DTT at pH 7 is −0.33V.

The pH during the pretreatment is selected such that the reducing compound has efficient reducing power. As well known in the art, DTT has for example efficient reducing activity at a pH value of 6.5 to 9.0 (preferably pH 7 to 8). TCEP is, for example redox active at a pH range of about 1.5 to about 8.5.

The concentration of the (protein) reducing agent can be varied and depends on the precise reducing agent used. As illustrated in the appended Examples for DTT the concentrations to be used for an efficient pretreatment can be determined by comparing the immunoassay signal for a sample comprising a detectable level of α-1,6-core-fucosylated AFP using different concentrations of (protein) reducing agent prior to conducting a immunoassay that is otherwise identical.

Exemplary, if DTT is used as (protein) reducing agent, the final concentration of DTT in the sample during the pretreatment may be from 0.1 mM to 100 mM, 0.1 mM to 60 mM, 0.1 mM to 50 mM or 0.1 mM to 47.8 mM. In embodiments, the concentration of DTT may be at least 0.2 mM, at least 0.3 mM, at least 0.5 mM or at least 1 mM. In embodiments, the DTT concentration may be at most 100 mM, at most 60 mM, at most 50 mM or at most 47.8 mM. In embodiments, the concentration of DTT may be 2.2 mM. In embodiments the concentration of DTT may be 2.3 mM. In embodiments, the DTT the final concentration of DTT in the mixture of pretreatment agent and sample may be from 0.6 mM to 107.8 mM. In embodiments, the DTT the final concentration of DTT in the mixture of pretreatment agent and sample may be from 0.7 mM to 107.8 mM.

The concentration of DTT in the pretreatment agent is higher than in the final mixture of the sample during the pretreatment. For instance, the pretreatment agent may be a 2-fold to 20-fold, or a 2-fold to 10-fold concentrate (e.g. a 2, 3, 4, 5, 6, 7, 8, 9, 10, 11, 12, 13, 14, 15, 16, 17, 18, 19 or 20 fold concentrate); i.e. stock solution.

The pretreatment agent used for pretreatment of the sample may further comprise a chelating agent, such as a chelating agent binding to ions. It has been found that such chelating agent improves stability of a pretreatment agent comprising a reducing agent such as DTT. In a particular embodiment the chelating agent may be a chelating agent binding to ions. In a more specific embodiment, the chelating agent may be a chelating agent binding to divalent ions.

Non-limiting examples for chelating agents for binding to (divalent) ions are aminopolycarboxylic acids like ethylenediaminetetraacetic acid (EDTA) and diethylenetriaminepentaacetic acid (DTPA), or porphyrins, polyamines, crown ethers, ethylene glycol-bis(R-aminoethyl ether)-N,N,N′,N′-tetraacetic acid (EGTA).

Further non-limiting examples for chelating agents are DTPA, EC, DMSA, EDTA, EGTA, Cy-EDTA, EDTMP, DTPA, CyDTPA, Cy2DTPA, BOPTA, DTPA-MA, DTPA-BA, DTPMP, DOTA, TRITA, TETA, DOTMA, DOTA-MA, HP-DO3A, pNB-DOTA, DOTP, DOTMP, DOTEP, DOTPP, DOTBzP, DOTPME, HEDP, DTTP, anN3 S triamidethiol, DADS, MAMA, DADT, N2S4 diamine tetrathiol, N2P2 dithiol-. Bisphosphine, 6-hydrazinonicotinic acid, propyleneamine oxime, tetraamine, cyclam, or a combination thereof.

In embodiments, the chelating agent is EDTA.

The concentrations of the chelating agent during the pretreatment of the sample can be varied and may depend on the precise chelating agent and reducing agent used. Useful concentration ranges can be determined by comparing the immunoassay signal for a distinct sample comprising a detectable level of α-1,6-core-fucosylated AFP using different concentrations of chelating agent.

For example, if EDTA is used, exemplary concentrations during the pretreatment of the sample are 0.1 mM to 50 mM, 0.1 mM to 20 mM, 0.1 mM to 10 mM 0.1 mM to 5 mM.

In embodiments, the pretreatment agent further comprises a buffer substance (also referred to as buffer herein). The buffer agent may be selected depending on the pH required for the protein reducing agent to reduce disulfide bonds.

In embodiments, the pretreatment agent comprises or consists of a protein reducing agent and a chelating agent for ions (e.g. divalent ions) and optionally a buffer.

In embodiments, the pretreatment agent comprises DTT and EDTA and optionally a buffer.

The pretreatment of the sample may be conducted for different times. For example, the pretreatment may be conducted from 1 min to 30 min, from 2 min to 20 min or 3 min to 10 min. In embodiments, the pretreatment may be conducted for 9 min.

In embodiments, the pretreatment may be an incubation of the sample with DTT at a pH 6.5 to 9.0 (preferably pH 7 to 8) for a predefined time (e.g. at least 2, 3, 4, 5, 6, 7, 8 or 9 min). Optionally also a chelating agent as described above may be present (e.g. EDTA). In embodiments, the pretreatment may also comprise addition of a buffer (e.g. Tris in concentrations of 10 mM to 200 mM, 30 mM to 150 mM, 50 mM to 130 mM, or 100 mM) to the sample adjusting the pH in the sample to above 7. Such pH ensures the reducing potential of DTT. Preferably, the pH may be adjusted to a pH between 7 and 10, 7 and 9 or 7 and 8.

After the pretreatment the concentration of DTT may be reduced such that the detection complex can still form.

In embodiments, the method of the ninth aspect may further comprise incubating the sample with a AFP-specific antibody that does not compete for binding to AFP with a 1,6-fucAFP antibody or antigen binding fragment of the invention. In these embodiments, the detection complex further comprises said AFP specific antibody, i.e. a sandwich comprising the 1,6-fucAFP antibody of the invention, the α-1,6-core-fucosylated AFP and the AFP-specific antibody that does not compete for binding to AFP with a 1,6-fucAFP antibody or antigen binding fragment of the invention. In the embodiments using a pretreatment, the AFP specific antibody shall be selected such that the antibody can still bind to AFP in the presence of the pretreatment agent.

A non-limiting example for an AFP-specific antibody that does not compete for binding with the antibodies of the invention is the commercially available anti-AFP antibody Tu11 (Roche Diagnostics Deutschland GmbH, material number: 11492080103).

Accordingly, antibodies that do not compete for binding to AFP with the antibodies of the invention may be antibodies or antigen binding fragments thereof that have the same epitope as Tu11 or compete for binding to AFP with the Tu11 antibody. As demonstrated by the appended examples, the Tu11 antibody is also compatible with the pretreatment of the sample according to the present invention.

In embodiments, the method of the ninth aspect may comprise digesting the α-1,6-core-fucosylated AFP comprised in the sample to glycopeptides and peptides (e.g. using proteases) prior to the incubation with the antibody or antigen binding fragment of the invention, said glycopeptides comprising partial AFP sequences comprising α-1,6-core-fucosylated Asn-251 of AFP (e.g. as described herein elsewhere). Optionally the agents for generating glycopeptides or peptides may be removed or inactivated before adding the antibody or antigen binding fragment of the invention. The presence or level of α-1,6-core-fucosylated AFP in the sample may then be determined based on detection of a detection complex comprising the partial AFP sequences comprising α-1,6-core-fucosylated Asn-251 of AFP and the antibody or antigen binding fragment of the invention.

In embodiments, the method according to the ninth aspect may be a heterogeneous immunoassay. In embodiments, the method according to the ninth aspect may be sandwich immunoassay (e.g. a heterogeneous sandwich immunoassay). In embodiments, the method according to the ninth aspect may be an immunohistochemistry (IHC) assay and the sample may be a tissue slide. In embodiments, the method according to the ninth aspect is a body fluid immunoassay, wherein the sample is a body fluid.

The sample used in the method according to the ninth aspect may be a tissue slide or a body fluid, such as, but not restricted to, a blood sample, cerebrospinal fluid, seminal fluid, saliva or urine. In embodiments, the sample is a blood sample, such as whole blood, serum or plasma. In embodiments, the sample is serum or plasma.

The method according to the ninth aspect is an in vitro method on samples that were previously obtained from a subject.

As known in the art, AFP-L3 is a useful marker, preferably in conjunction with other markers for detection of early hepatocellular carcinoma (HCC) and α-1,6-core-fucosylated AFP is the core component of AFP-L3 Accordingly, provided herein is an in vitro method for aiding in the detection or detection of HCC comprising
a) determining the level of α-1,6-core-fucosylated AFP in a sample obtained from a subject using a method according to the ninth aspect;
b) comparing the determined level of α-1,6-core-fucosylated AFP in the sample to a reference level for α-1,6-core-fucosylated AFP; and
c) aiding in the detection of HCC or detecting HCC based on the comparison.

In particular AFP-L3 and consequently α-1,6-core-fucosylated AFP is also frequently used in combination with other biomarkers and/or clinical or demographic information in scores for detecting HCC- An exemplary score is the so-called GALAD score, involving gender, age, AFP-L3, AFP and DCP (also known as PIVKA-II).

Consequently herein provided is also an in vitro method for aiding in the detection of or detecting HCC comprising
a) determining the level of α-1,6-core-fucosylated AFP in a sample obtained from a subject using a method according to the ninth aspect of the invention;
b) calculating a score for HCC detection taking into account the determined level of α-1,6-core-fucosylated AFP; and
c) aiding in the detection of HCC or detecting HCC based on the calculated score (e.g. by comparison to a reference value for said score indicative for HCC).

The score for HCC detection may take into account the level of at least one additional biomarker for HCC detection (e.g. AFP or DCP). Accordingly, the method may further comprise determining or receiving the level of said at least one additional HCC biomarker in the sample or another sample taken from the subject (e.g. at the same time).

The score for HCC detection may additionally or alternatively take into account a clinical parameter (e.g. ultrasound data) or demographic information (e.g. age and/or sex) of the subject. Accordingly, the method may further comprise receiving said clinical parameter or the said demographic information of the subject.

In a particular embodiment, the score for HCC detection may comprise the level of AFP and the level of DCP in the sample, and the gender and age of the subject, i.e. may be a GALAD score. Accordingly, the method may further comprise determining or receiving the level of AFP and the level of DCP (PIVKA-II) in the sample or another sample taken from the subject (e.g. at the same time) and receiving the age and gender of the subject.

Also provided herein is a computer-implemented method for detecting HCC comprising
a) receiving the level of α-1,6-core-fucosylated AFP in a sample obtained from a subject using a method according to the ninth aspect of the invention;
b) calculating a score for HCC detection taking into account the determined level of α-1,6-core-fucosylated AFP; and
c) aiding in the detection of HCC or detecting HCC based on the calculated score (e.g. by comparison to a reference value for said score indicative for HCC).

The embodiments of the aforementioned in vitro method for detecting HCC using a score taking into account the determined level of α-1,6-core-fucosylated AFP in a sample obtained from a subject using a method according to the ninth aspect of the invention apply mutatis mutandis with the only exception that levels of further biomarker are received and not determined.

In a tenth aspect, the present invention provides for a pretreatment reagent for use in an immunoassay for the detection of α-1,6-core-fucosylated AFP (e.g. using the antibodies or antigen binding fragments of the invention), which comprises a reducing agent.

The term pretreatment reagent is used herein interchangeably with the terms “pretreatment agent”, “pretreating agent” and “pretreating reagent” and relates to a pretreatment composition that is applied to a sample (e.g. a blood sample, such as serum or plasma) so as to facilitate the binding of the 1,6fucAFP antibodies to the α-1,6-core-fucosylated AFP comprised in the sample.

In embodiments, the pretreatment reagent of the tenth aspect of the present disclosure the reducing agent may be protein reducing agent. In other words, the pretreatment reagent may comprise or consist of a protein reducing agent.

A protein reducing agent is a reducing agent that can reduce/break down disulfide bonds within a protein/polypeptide chains or between proteins/polypeptide chains.

Non-limiting examples for reducing agents or protein reducing agents are dithiothreitol (DTT), tris(2-carboxyethyl)phosphine (TCEP), β-mercaptoethanol and dithiobutylamine (DTBA). In embodiments, the reducing agent or protein reducing agent may be selected from dithiothreitol (DTT), tris(2-carboxyethyl)phosphine (TCEP) and dithiobutylamine (DTBA). In embodiments, the reducing agent or protein reducing agent may be selected from dithiothreitol (DTT) and tris(2-carboxyethyl)phosphine (TCEP). In embodiments, the reducing agent or protein reducing agent may be dithiothreitol (DTT).

In embodiments, the reducing agent may be a protein reducing agent having a redox potential at a pH of 7 which is 50% to 150% of the redox potential of DTT at a pH of 7. The redox potential of DTT at pH 7 is −0.33V.

The pH of the pretreatment reagent can be selected in a broad range and may be varied dependent on the components comprised in the pretreatment agent. Depending on the needs the pH may be selected such that the reduction potential is as high as possible or can be selected such that the stability (shelf life) of the reducing agent is preserved. In embodiments, the pH may be selected such that the pH of the sample to which the pretreatment reagent is added is adjusted to a pH that allows reduction activity of the reducing agent after being added to the sample.

Exemplary, the reducing agent is DTT and the pH of the pretreatment agent is selected below pH 6.5 (e.g. pH 4 to 6.5, pH 5 to 6.5, pH 5 to 6.5, or pH 5.5 to 6.5). pH values in this range stabilize DTT, i.e. allow for a longer storage of the pretreatment agent.

The concentration of the (protein) reducing agent in the pretreatment agent can be varied and depends on the precise reducing agent used and the factor by which the pretreatment agent is concentrated over the final use concentration. Moreover, stability of reducing agents may depend on the precise concentration. A skilled person can select the concentration of the reducing agent in the pretreatment agent such that it facilitates the binding of the 1,6fucAFP antibody or antigen binding fragment to the α-1,6-core-fucosylated AFP in a sample (e.g. a blood sample such as plasma or serum). For instance, the signal of an immunoassay using a control sample comprising α-1,6-core-fucosylated AFP and using different concentrations of the reducing agent in the pretreatment agent may be compared and the concentration with the highest immunoassay signal may be selected. Exemplary assays are described in the appended Examples.

Exemplary, if DTT is used as reducing agent, the concentration in the pretreatment agent may be selected such that a final concentration of DTT in the sample during the pretreatment from 0.1 mM to 100 mM, 0.1 mM to 60 mM, 0.1 mM to 50 mM or 0.1 mM to 47.8 mM can be achieved (e.g. without diluting the sample by the addition of the pretreatment reagent by more than 2-fold, 3-fold, 4-fold, 5-fold or 10-fold). In embodiments, the concentration in the pretreatment agent may be selected such that a final concentration of DTT in the sample during the pretreatment of at least 0.2 mM, at least 0.3 mM, at least 0.5 mM or at least 1 mM can be achieved (e.g. without diluting the sample by the addition of the pretreatment reagent by more than 2-fold, 3-fold, 4-fold, 5-fold or 10-fold). In embodiments, the DTT the concentration in the pretreatment agent may be selected such that a final concentration of DTT in the sample during the pretreatment of at most 100 mM, at most 60 mM, at most 50 mM or at most 47.8 mM can be achieved (e.g. without diluting the sample by the addition of the pretreatment reagent by more than 2-fold, 3-fold, 4-fold, 5-fold or 10-fold). In embodiments, the concentration in the pretreatment agent may be selected such that a final concentration of DTT in the sample during the pretreatment of 2.2 mM can be achieved (e.g. without diluting the sample by the addition of the pretreatment reagent by more than 2-fold, 3-fold, 4-fold, 5-fold or 10-fold). In embodiments, the concentration in the pretreatment agent may be selected such that a final concentration of DTT in the sample during the pretreatment of 2.3 mM can be achieved (e.g. without diluting the sample by the addition of the pretreatment reagent by more than 2-fold, 3-fold, 4-fold, 5-fold or 10-fold). In embodiments, the concentration of DTT in the pretreatment agent may be selected such that a final concentration of DTT in the sample during the pretreatment of 0.7 mM to 107.8 mM can be achieved (e.g. without diluting the sample by the addition of the pretreatment reagent by more than 2-fold, 3-fold, 4-fold, 5-fold or 10-fold). In embodiments, the concentration of DTT in the pretreatment agent may be selected such that a final concentration of DTT in the sample during the pretreatment from 0.6 mM to 107.8 mM can be achieved (e.g. without diluting the sample by the addition of the pretreatment reagent by more than 2-fold, 3-fold, 4-fold, 5-fold or 10-fold).

Exemplary concentrations of a reducing agent (e.g. DTT) in the pretreatment agent may be from 0.1 mM to 150 mM, 0.1 mM to 110 mM, 0.5 mM to 100 mM, 1 mM to 50 mM, 1 mM to 20 mM or 1 mM to 10 mM. In embodiments, the concentration of a reducing agent (e.g. DTT) in the pretreatment agent may be 1 mM to 10 mM (e.g. 10 mM). In embodiments, the concentration of a reducing agent (e.g. DTT) in the pretreatment agent may be from 3 mM to 110 mM.

In embodiments, the pretreatment agent may be a 2-fold to 20-fold, or a 2-fold to 10-fold concentrate (e.g. a 2, 3, 4, 5, 6, 7, 8, 9, 10, 11, 12, 13, 14, 15, 16, 17, 18, 19 or 20 fold concentrate); i.e. stock solution. In embodiments, the pretreatment agent may be a 4 to 8-fold concentrate.

The pretreatment agent according to the tenth aspect of the present invention may further comprise a chelating agent, such as a chelating agent binding to ions. It has been found that addition of such chelating agent improves stability of the pretreatment reagent comprising a reducing agent such as DTT. In a particular embodiment, the chelating agent may be a chelating agent binding to ions. In a more specific embodiment, the chelating agent may be a chelating agent binding to divalent ions.

Non-limiting examples for chelating agents for binding to (divalent) ions are aminopolycarboxylic acids like ethylenediaminetetraacetic acid (EDTA) and diethylenetriaminepentaacetic acid (DTPA), or porphyrins, polyamines, crown ethers, or ethylene glycol-bis(β-aminoethyl ether)-N,N,N′,N′-tetraacetic acid (EGTA).

Further non-limiting examples for chelating agents are DTPA, EC, DMSA, EDTA, EGTA, Cy-EDTA, EDTMP, DTPA, CyDTPA, Cy2DTPA, BOPTA, DTPA-MA, DTPA-BA, DTPMP, DOTA, TRITA, TETA, DOTMA, DOTA-MA, HP-DO3A, pNB-DOTA, DOTP, DOTMP, DOTEP, DOTPP, DOTBzP, DOTPME, HEDP, DTTP, anN3 S triamidethiol, DADS, MAMA, DADT, N2S4 diamine tetrathiol, N2P2 dithiol-. Bisphosphine, 6-hydrazinonicotinic acid, propyleneamine oxime, tetraamine, cyclam, or a combination thereof.

In embodiments, the chelating agent is EDTA.

The concentration of the chelating agent in the pretreatment agent can be varied and depends on the precise chelating agent used and the factor by which the pretreatment agent is concentrated over the final use concentration. A skilled person can select the concentration of the chelating agent in the pretreatment agent such that it facilitates the binding of the 1,6fucAFP antibody or antigen binding fragment to the α-1,6-core-fucosylated AFP in a sample (e.g. a blood sample such as plasma or serum). For instance, the signal of an immunoassay using a control sample comprising α-1,6-core-fucosylated AFP and using different concentrations of the chelating agent in the pretreatment agent may be compared and the concentration with the highest immunoassay signal and/or a higher storage stability may be selected. Exemplary assays are described in the appended Examples.

For example, if EDTA is used as chelating agent, exemplary concentrations during the pretreatment of the sample are 0.1 mM to 50 mM, 0.1 mM to 20 mM, 0.1 mM to 10 mM 0.1 mM to 5 mM (e.g. 4 mM).

In embodiments, the pretreatment agent further comprises a buffer substance (also referred to as buffer herein). The buffer agent may be selected depending on the pH required for the protein reducing agent to reduce disulfide bonds. For example, Tris, Hepes or citrate may be used.

In embodiments, the pretreatment agent comprises or consists of a protein reducing agent and a chelating agent for ions (e.g. divalent ions) and optionally a buffer.

In embodiments, the pretreatment agent comprises DTT and EDTA and optionally a buffer. Said pretreatment agent may be 4 to 5 fold concentrate and may comprise 8 to 12 mM DTT (e.g. 10 mM) and 1 to 5 mM EDTA (e.g. 4.5 mM). The pH of the pretreatment reagent may be pH 5.5 or lower (e.g. pH 4 to 5.5, or pH 5.5).

In a specific embodiment, a set of pretreatment reagents for use in an immunoassay for the detection of α-1,6-core-fucosylated AFP (e.g. using the antibodies or antigen binding fragments of the invention) is provided. Said set of pretreatment reagents may comprise a first and a second pretreatment composition each provided in separate containers. The first pretreatment composition may be any of the pretreatment agents as defined in the context of the tenth aspect and comprises a reducing agent. The pH of the first pretreatment composition is preferably selected such that the stability of the components is high.

The second pretreatment composition may comprise a buffer and may have a pH and buffer concentration that allows reduction activity of the reducing agent if both the first and the second pretreatment composition are added to a sample.

In a particular embodiment, the first pretreatment composition may comprise DTT and EDTA. Said pretreatment agent may be 4 to 5 fold concentrate and may comprise 8 to 12 mM DTT (e.g. 10 mM) and 1 to 5 mM EDTA (e.g. 2 mM). The pH of the pretreatment reagent may be pH 5.5 or lower (e.g. pH 4 to 5.5, or pH 5.5). The second pretreatment composition may comprise a buffer and may have a pH of 6.5 to 9, e.g. 7.5 to 8.5. The buffer concentration may be from 10 mM to 150 mM, 20 mM to 100 mM, or 30 mM to 80 mM (e.g. 100 mM). The buffer may be a buffer with a pKa of 7 to 9, e.g. 7.5 to 8.5. In embodiments, the buffer may be Tris (e.g. pH 8.5).

In an eleventh aspect, the present invention provides for a kit comprising a 1,6fucAFP antibody or antigen binding fragment of the invention. The kit may be a kit for detection and/or quantification of α-1,6-core-fucosylated AFP or a partial AFP sequence comprising said α-1,6-core-fucosylation in a sample (e.g. a blood sample such as plasma or serum). The kit may be a kit for detection and/or quantification of α-1,6-core-fucosylated AFP in a sample (e.g. a blood sample such as plasma or serum).

In embodiments, the kit may be an immunoassay kit, such as a kit for a sandwich immunoassay (e.g. a heterogeneous sandwich immunoassay).

In a particular embodiment, the kit according to the eleventh aspect of the invention further comprises a pretreatment agent of the invention (see above) or a set of pretreatment agents (see above). In embodiments, the antibody or antigen binding fragment and the pretreatment reagent are provided in separate containers.

As demonstrated in the appended example especially the combination of a 1,6fucAFP antibody or antigen binding fragment of the invention and a pretreatment agent of the invention allows for detection and quantification of native α-1,6-core-fucosylated AFP with high signals using an immunoassay, e.g. an immunoassay as described in the context of the invention.

The present invention in particular also relates to the following items:
1. A monoclonal antibody or antigen binding fragment thereof specifically binding to α-1,6-core-fucosylated alpha-fetoprotein (AFP) or a partial sequence of AFP comprising said α-1,6-core-fucosylation.
2. The monoclonal antibody or antigen binding fragment according to item 1, which comprises
   (i) a heavy chain variable domain (VH) comprising a CDR-H1 having the amino acid sequence of SEQ ID NO: 3 or a variant thereof modified by one amino acid substitution; a CDR-H2 having the amino acid sequence of SEQ ID NO: 4 or 5 or a variant of SEQ ID NO: 4 or 5 modified by at most two amino acid substitutions; and a CDR-H3 having the amino acid sequence of SEQ ID NO: 6 or a variant thereof modified by one amino acid substitution; and
   (ii) a light chain variable domain (VL) comprising a CDR-L1 having the amino acid sequence of SEQ ID NO: 7 or 8 or a variant of SEQ ID NO: 7 or 8 modified by at most two amino acid substitutions; a CDR-L2 having the amino acid sequence of SEQ ID NO: 9 or a variant thereof modified by one amino acid substitution; and a CDR-L3 having the amino acid sequence of SEQ ID NO: 10 or a variant thereof modified by one amino acid substitution.
3. The monoclonal antibody or antigen binding fragment according to item 1 or 2, which comprises
   (i) a heavy chain variable domain (VH) comprising a CDR-H1 consisting of the amino acid sequence of SEQ ID NO: 3 or a variant thereof modified by one amino acid substitution; a CDR-H2 consisting of the amino acid sequence of SEQ ID NO: 4 or 5 or a variant of SEQ ID NO: 4 or 5 modified by at most two amino acid substitutions; and a CDR-H3 consisting of the amino acid sequence of SEQ ID NO: 6 or a variant thereof modified by one amino acid substitution; and
   (ii) a light chain variable domain (VL) comprising a CDR-L1 consisting of the amino acid sequence of SEQ ID NO: 7 or 8 or a variant of SEQ ID NO: 7 or 8 modified by at most two amino acid substitutions; a CDR-L2 consisting of the amino acid sequence of SEQ ID NO: 9 or a variant thereof modified by one amino acid substitution; and a CDR-L3 consisting of the amino acid sequence of SEQ ID NO: 10 or a variant thereof modified by one amino acid substitution.
4. The monoclonal antibody or antigen binding fragment according to item 2 or 3, wherein the total sum of the amino acid substitutions in all CDR sequences is at most 8, at most 7, at most 6, at most 5, at most 4, at most 3, or at most 2.
5. The monoclonal antibody or antigen binding fragment according to any one of items 1 to 4, which comprises
   (i) a heavy chain variable domain (VH) comprising a CDR-H1 having the amino acid sequence of SEQ ID NO: 3; a CDR-H2 having the amino acid sequence of SEQ ID NO: 4 or 5 or a variant of SEQ ID NO: 4 or 5 modified by one amino acid substitution; and a CDR-H3 having the amino acid sequence of SEQ ID NO: 6; and
   (ii) a light chain variable domain (VL) comprising a CDR-L1 having the amino acid sequence of SEQ ID NO: 7 or 8 or a variant of SEQ ID NO: 7 or 8 modified by one amino acid substitution; a CDR-L2 having the amino acid sequence of SEQ ID NO: 9; and a CDR-L3 having the amino acid sequence of SEQ ID NO: 10.
6. The monoclonal antibody or antigen binding fragment according to any one of item 1 to 5, which comprises
   (i) a heavy chain variable domain (VH) comprising a CDR-H1 consisting of the amino acid sequence of SEQ ID NO: 3; a CDR-H2 consisting of the amino acid sequence of SEQ ID NO: 4 or 5 or a variant of SEQ ID NO: 4 or 5 modified by one amino acid substitution; and a CDR-H3 consisting of the amino acid sequence of SEQ ID NO: 6; and
   (ii) a light chain variable domain (VL) comprising a CDR-L1 consisting of the amino acid sequence of SEQ ID NO: 7 or 8 or a variant of SEQ ID NO: 7 or 8 modified by one amino acid substitution; a CDR-L2 consisting of the amino acid sequence of SEQ ID NO: 9; and a CDR-L3 consisting of the amino acid sequence of SEQ ID NO: 10.
7. The monoclonal antibody or antigen binding fragment according to any one of items 2 to 6, wherein the amino acid substitution(s) in the variant of SEQ ID NOs: 4 or 5 comprise an amino acid substitution at position 5 of SEQ ID NO: 4 or at position 4 of SEQ ID NO: 5 and/or wherein the amino acid substitution(s) in the variants of SEQ ID NOs: 7 or 8 comprise an amino acid substitution at position 7 of SEQ ID NO: 7 or 8, respectively.
8. The monoclonal antibody or antigen binding fragment according to any one of items 1 to 7, wherein the amino acid substitution(s) in the variant of SEQ ID NOs: 4 or 5 comprise a conservative amino acid substitution at position 5 of SEQ ID NO: 4 or a conservative amino acid substitution at position 4 of SEQ ID NO: 5 and/or wherein the amino acid substitution(s) in the variant of SEQ ID NOs: 7 or 8 comprise a conservative amino acid substitution at position 7 of SEQ ID NO: 7 or 8.
9. The monoclonal antibody or antigen binding fragment according to any one of items 2 to 7, wherein all of said amino acid substitutions are conservative or highly conservative amino acid substitutions.
10. The monoclonal antibody or antigen binding fragment according to any one of item 2 to 8, wherein the amino acid in the variant of SEQ ID NOs: 4 or 5 corresponding to the amino acid at position 5 of SEQ ID NO: 4 or position 4 of SEQ ID NO: 5 is serine or asparagine; and/or wherein the amino acid in the variant SEQ ID NO: 7 or 8 corresponding to the amino acid position 7 of SEQ ID NO: 7 or 8 is serine or glycine.
11. The monoclonal antibody or antigen binding fragment according to any one of items 7, 8 and 10, wherein all amino acid substitutions other than at position 5 of SEQ ID NO: 4 or at position 4 of SEQ ID NO: 5 and/or at position 7 of SEQ ID NO: 7 or 8 are conservative or highly conservative amino acid substitutions.
12. The monoclonal antibody or antigen binding fragment according to any one of items 8, 9 or 11 wherein the conservative substitution(s) is/are the substitution of an amino acid with another amino acid selected from its same group, wherein the groups of amino acids are
   a) the nonpolar, hydrophobic amino acids consisting of Gly, Ala, Val, Leu, Ile, Phe, Tyr, Trp, and Met;
   b) the polar, neutral amino acids consisting of Ser, Thr, Asn, and Gln;
   c) the positively charged, basic amino acids consisting of Arg, Lys, and His, and
   d) the negatively charged, acidic amino acids consisting of Asp and Glu
   wherein if Cys is to be conservatively substituted, it is substituted with Ser or Ala, and wherein if Pro is to be conservatively substituted it is substituted with Ala.
13. The monoclonal antibody or antigen binding fragment antibody according to item 9, 11 or 12, wherein said highly conservative amino acid substitution is selected from
   a) substitution of Ala with Val, Leu, Ile or Gly;
   b) substitution of Arg with Lys;
   c) substitution of Asn with Gln;
   d) substitution of Asp with Glu;
   e) substitution of Cys with Ser;
   f) substitution of Gln with Asn;
   g) substitution of Glu with Asp;
   h) substitution of Gly with Ala;
   i) substitution of His with Arg;
   j) substitution of Ile with Leu, Val or Ala;
   k) substitution of Leu with Ile, Val or Ala;
   l) substitution of Lys with Arg;
   m) substitution of Met with Leu, Ile or Val;
   n) substitution of Phe with Tyr or Trp;
   o) substitution of Pro with Ala;
   p) substitution of Ser with Thr;
   q) substitution of Thr with Ser;
   r) substitution of Trp with Phe or Tyr;
   s) substitution of Tyr with Phe or Trp; and
   t) substitution of Val with Leu, Ile or Ala.
14. The monoclonal antibody or antigen binding fragment according to any one of items 1 to 13 comprising
   (i) a heavy chain variable domain (VH) having an amino acid sequence with at least 80%, at least 85%, at least 90%, at least 95%, or preferably at least 97.5% sequence identity to SEQ ID NO:11 or 12; and
   (ii) a light chain variable domain (VL) having an amino acid sequence with at least 80%, at least 85%, at least 90%, at least 95%, or preferably at least 97.3% sequence identity to SEQ ID NO: 13 or 14.
15. The monoclonal antibody or antigen binding fragment according to item 14, wherein the CDR-H1, CDR-H2, CDR-H3, CDR-L1, CDR-L2 and CDR-L3 are as defined in any one of items 2 to 13.
16. The monoclonal antibody or antigen binding fragment according to any one of items 1 to 15, wherein the heavy chain variable domain (VH) comprises framework regions (FW) and has the structure of:
   FW-H1-CDR-H1-FW-H2-CDR-H2-FW-H3-CDR-H3-FW-H4
wherein the FW-H1 has the amino acid sequence of SEQ ID NO: 15 o or a variant thereof having at least 60%, preferably at least 70%, even more preferably at least 80%, even more preferably at least 85%, even more preferably at least 90%, even more preferably at least 95%, even more preferably 98% and even more preferably 99% thereto;
wherein the FW-H2 has the amino acid sequence of SEQ ID NO: 16 or 17 or a variant of SEQ ID NO: 16 or 17 having at least 60%, preferably at least 70%, even more preferably at least 80%, even more preferably at least 85%, even more preferably at least 90%, even more preferably at least 95%, even more preferably 98% and even more preferably 99% thereto;
wherein the FW-H3 has the amino acid sequence of SEQ ID NO: 18 or a variant thereof having at least 60%, preferably at least 70%, even more preferably at least 80%, even more preferably at least 85%, even more preferably at least 90%, even more preferably at least 95%, even more preferably 98% and even more preferably 99% thereto; and
wherein the FW-H4 has the amino acid sequence of SEQ ID NO: 19 or a variant thereof having at least 60%, preferably at least 70%, even more preferably at least 80%, even more preferably at least 85%, even more preferably at least 90%, even more preferably at least 95%, even more preferably 98% and even more preferably 99% thereto.
17. The monoclonal antibody or antigen binding fragment according to any one of items 1 to 16, wherein the light chain variable domain (VL) comprises framework regions (FW) and has the structure of:
   FW-L1-CDR-L1-FW-L2-CDR-L2-FW-L3-CDR-L3-FW-L4
wherein the FW-L1 has the amino acid sequence of SEQ ID NO: 20 o or a variant thereof having at least 60%, preferably at least 70%, even more preferably at least 80%, even more preferably at least 85%, even more preferably at least 90%, even more preferably at least 95% thereto, even more preferably 98% and even more preferably 99% thereto;
wherein the FW-L2 has the amino acid sequence of SEQ ID NO: 21 or 22 or a variant of SEQ ID NO: 21 or 22 having at least 60%, preferably at least 70%, even more preferably at least 80%, even more preferably at least 85%, even more preferably at least 90%, even more preferably at least 95%, even more preferably 98% and even more preferably 99% thereto;
wherein the FW-L3 has the amino acid sequence of SEQ ID NO: 23 or a variant thereof having at least 60%, preferably at least 70%, even more preferably at least 80%, even more preferably at least 85%, even more preferably at least 90%, even more preferably at least 95%, even more preferably 98% and even more preferably 99% thereto; and
wherein the FW-L4 has the amino acid sequence of SEQ ID NO: 24 or a variant thereof having at least 60%, preferably at least 70%, even more preferably at least 80%, even more preferably at least 85%, even more preferably at least 90%, even more preferably at least 95%, even more preferably 98% and even more preferably 99% thereto.
18. The monoclonal antibody or antigen binding fragment according to any one of items 1 to 17 comprising
   (i) a heavy chain variable domain (VH) having the amino acid sequence SEQ ID NO: 11 or 12; and
   (ii) a light chain variable domain (VL) having the amino acid sequence of SEQ ID NO: 13 or 14.
19. The monoclonal antibody or antigen binding fragment according to any one of items 1 to 18, wherein said partial sequence of AFP comprises or consists of SEQ ID NO: 2.
20. The monoclonal antibody or antigen binding fragment according to any one of items 1 to 19, wherein said α-1,6-core-fucosylated AFP or the partial sequence of AFP comprising said α-1,6-core-fucosylation comprises or consists of the glycopeptide of Formula I

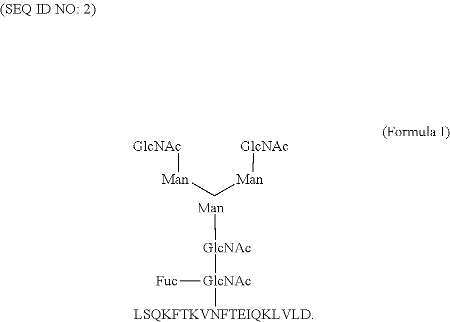

21. The monoclonal antibody or antigen binding fragment according to any one of item 1 to 20, wherein said α-1,6-core-fucosylated AFP or the partial sequence of

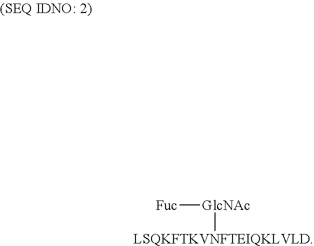

AFP comprising said α-1,6-core-fucosylation comprises or consists of the glycopeptide of Formula II
   (Formula II).
22. The monoclonal antibody or antigen binding fragment according to any one of items 1 to 21, wherein said antibody or antigen binding fragment discriminates between (i) said α-1,6-core-fucosylated AFP or the partial sequence of AFP comprising said α-1,6-core-fucosylation, and (ii) AFP or a partial sequence thereof lacking the α-1,6-core-fucose residue.
23. The monoclonal antibody or antigen binding fragment according to any one of items 20 to 22, wherein said partial sequence of AFP comprises or consists of SEQ ID NO: 2.
24. The monoclonal antibody or antigen binding fragment according to item 22 or 23, wherein said AFP or a partial sequence thereof lacking the α-1,6-core-fucose residue comprises of consists of the glycopeptide of Formula III,

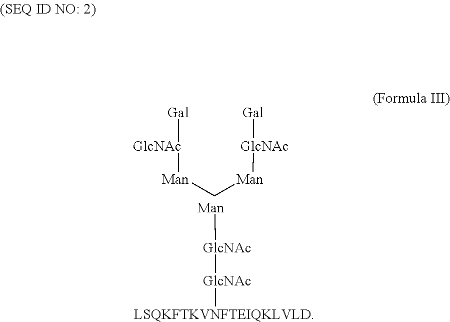

25. The monoclonal antibody or antigen binding fragment according any one of items 22 to 24, which antibody or antigen binding fragment binds to (i) said α-1,6-core-fucosylated AFP or the partial AFP sequence comprising the α-1,6-core-fucosylation with an equilibrium dissociation constant (K_{D}) that is at least 10 fold, at least 20 fold, at least 50 fold or at least 100 fold, at least 150 fold, at least 180 fold or at least 245 fold lower than the K_{D} for the binding to (ii) said AFP lacking the α-1,6-core-fucose residue or the partial sequence of AFP lacking the α-1,6-core-fucose residue, wherein the binding affinity for (i) and (ii) are determined under the same conditions.
26. The monoclonal antibody or antigen binding fragment according to any one of items 1 to 25, wherein said antibody or antigen binding fragment discriminates between (i) said α-1,6-core-fucosylated AFP or the partial sequence of AFP comprising said α-1,6-core-fucosylation, and (ii) the α-1,6-core-fucosylated glycan of formula (IV),

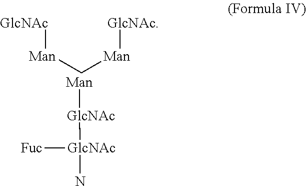

27. The monoclonal antibody or antigen binding fragment according to item 26, which antibody or antigen binding fragment binds to (i) said α-1,6-core-fucosylated AFP or the partial AFP sequence comprising the α-1,6-core-fucosylation with an equilibrium dissociation constant (K_{D}) that is at least 10 fold, at least 20 fold, at least 50 fold or at least 100 fold, at least 150 fold, at least 180 fold, at least 245 fold lower than the K_{D} for the binding to (ii) said α-1,6-core-fucosylated glycan of formula (IV), wherein the binding affinity for (i) and (ii) are determined under the same conditions.
28. The monoclonal antibody or antigen binding fragment according to any one of items 1 to 27, wherein said antibody or antigen binding fragment discriminates between (i) said α-1,6-core-fucosylated AFP or the partial sequence of AFP comprising said α-1,6-core-fucosylation, and (ii) a AFP peptide of SEQ ID NO: 2 or 25.
29. The monoclonal antibody or antigen binding fragment according to item 28, which antibody or antigen binding fragment binds to (i) said α-1,6-core-fucosylated AFP or the partial AFP sequence comprising the α-1,6-core-fucosylation with an equilibrium dissociation constant (K_{D}) that is at least 10 fold, at least 20 fold, at least 50 fold, at least 100 fold, at least 500 fold, at least 1000 fold or at least 5000 fold lower than the K_{D} for the binding to (ii) said a AFP peptide of SEQ ID NO: 2 or 25, wherein the binding affinity for (i) and (ii) are determined under the same conditions.
30. The monoclonal antibody or antigen binding fragment according to any one of items 1 to 29, wherein said antibody or antigen binding fragment discriminates between the glycopeptide according to Formula I and both the glycopeptide of Formula III and the core-fucosylated glycan of Formula IV.
31. The monoclonal antibody or antigen binding fragment according to any one of items 1 to 30, wherein said antibody or antigen binding fragment discriminates between the glycopeptide according to Formula I and all of the following three: the glycopeptide of Formula III, the core-fucosylated glycan of Formula IV and the AFP peptide of SEQ ID NO: 2 or 25.
32. The monoclonal antibody or antigen binding fragment according to any one of items 1 to 31 that binds to the glycopeptide of Formula I with a K_{D} of 100 nM or less, 20 nM or less, preferably 10 nM or less, more preferably 3.1 nM or less or 2.5 nM or less (optionally if the K_{D} is measured by affinity in solution surface plasmon resonance spectroscopy 0.9 nM or less or 0.4 nM or less), optionally wherein the K_{D} is measured at 37° C.
33. The monoclonal antibody or antigen binding fragment antibody according to any one of items 1 to 32, wherein the association rate constant kₐ for the glycopeptide of Formula I is at least 2.0*10⁴ M⁻¹s⁻¹, in embodiments at least 10⁵ M⁻¹s⁻¹, in embodiments at least 2.5*10⁵ M⁻¹s⁻¹, in embodiments at least 6.0*10⁵ M⁻¹s⁻¹ and in embodiments at least 1.0*10⁶ M⁻¹s⁻¹, optionally wherein the kₐ is measured at 37° C.
34. The monoclonal antibody or antigen binding fragment antibody according to any one of items 1 to 33, wherein the dissociation rate constant k_{d} for the glycopeptide of Formula I is at most 1.2*10⁻² s⁻¹, at most 8.0*10⁻³ s⁻¹, at most 7.3*10⁻³ s⁻¹, at most 3.2*10⁻³ s⁻¹ or at most 1.5*10⁻³ s⁻¹, optionally wherein the k_{d} is measured at 37° C.
35. The monoclonal antibody or antigen binding fragment according to any one of items 1 to 34 (if mentioning one or more K_{D} and/or kₐ and/or k_{d}), wherein the K_{D} and/or kₐ and/or k_{d} value(s) is/are determined by surface plasmon resonance spectroscopy.
36. The monoclonal antibody or antigen binding fragment according to item 35, wherein said surface plasmon resonance spectroscopy comprises capturing the monoclonal antibody or antigen binding fragment on a C1 sensor chip and injecting the respective glycopeptide or peptide (e.g. glycopeptide of Formula I) as analyte, optionally wherein said determination is conducted at a temperature of 37° C., optionally using HBS-ET pH 7.4 (10 mM HEPES pH 7.4, 150 mM NaCl, 3 mM EDTA, 0.05% w/v Tween 20®) as system buffer and system buffer supplemented with 1 mg/ml carboxymethyldextran as sample buffer.
37. The monoclonal antibody or antigen binding fragment according to item 35 or 36, wherein said K_{D} and/or kₐ and/or k_{d} value(s) is/are determined at 37° C.
38. The monoclonal antibody or antigen binding fragment according to any one of items 35 to 37, wherein the surface plasmon resonance spectroscopy is performed with a Biacore T200 or the 8K instrument.
39. The monoclonal antibody or antigen binding fragment according to any one of items 35 to 38, wherein the determination of said K_{D} and/or said kₐ comprises fitting the surface plasmon resonance data using a Langmuir fitting model, optionally with Rₘₐₓ global.
40. The monoclonal antibody or antigen binding fragment according to any one of items 35, wherein said K_{D} is determined by an affinity in solution method.
41. The monoclonal antibody or antigen binding fragment according to item 40, wherein said affinity in solution method comprises a competition for the binding of the antibody or antigen-binding fragment with a chip bound analyte and the same analyte in solution.
42. The monoclonal antibody or antigen binding fragment according to any one of items 1 to 41, which binds to the glycopeptide of Formula I with a K_{D} that is less than 10, 8, 6, 4, or 2 times the K_{D} of a rabbit IgG antibody comprising the heavy chain variable domain of SEQ ID NO: 11 and a light chain variable domain of SEQ ID NO: 13 towards the glycopeptide of Formula I, wherein the K_{D} values are measured under the same conditions and using the same method.
43. The monoclonal antibody or antigen binding fragment according to any one of items 1 to 42, which binds to the glycopeptide of Formula I with a K_{D} that is less than 10, 8, 6, 4, or 2 times the K_{D} of a rabbit IgG antibody comprising the heavy chain variable domain of SEQ ID NO: 12 and a light chain variable domain of SEQ ID NO: 14 towards the glycopeptide of Formula I, wherein the K_{D} values are measured under the same conditions and using the same method.
44. The monoclonal antibody or antigen binding fragment according to any one of items 1 to 43, which binds to α-1,6-core-fucosylated alpha-fetoprotein (AFP) that has been pretreated with a pretreatment agent of any one of 74 to 78.
45. The monoclonal antibody or antigen binding fragment according to any one of items 1 to 43, which binds to α-1,6-core-fucosylated alpha-fetoprotein (AFP) that has been pretreated with a pretreatment agent of any one of 74 to 78 better than without said pretreatment.
46. A polynucleotide or a set of polynucleotides encoding
   (i) the heavy chain or heavy chain variable domain of the monoclonal antibody or antigen binding fragment according to any one of items 1 to 45, and/or
   (ii) the light chain or light chain variable domain of the monoclonal antibody or antigen binding fragment according to any one of items 1 to 45.
47. A vector comprising the polynucleotide or the set of polynucleotides according to item 46.
48. A host cell comprising the polynucleotide or the set of polynucleotides according to item 46, or the vector according to item 47.
49. The host cell according to item 48 that is a prokaryotic cell or a eukaryotic cell.
50. The host cell according to item 48 that is a eukaryotic cell, wherein said cell is a CHO cell.
51. A method of producing the monoclonal antibody or antigen binding fragment according to any one of items 1 to 45 comprising culturing the host cell according to any one of items 48 to 50 and isolating said antibody or antigen binding fragment.
52. An antibody according to any one of items 1 to 46 obtainable by the method of item 51.
53. A composition comprising the antibody or antigen binding fragment according to any one of items 1 to 45 and 52, the polynucleotide or set of polynucleotides according to item 46, the vector according to item 47, or the host cell according to any one of items 48 to 50.
54. A composition comprising the antibody or antigen binding fragment according to any one of items 1 to 45 and 52 that is a diagnostic composition.
55. Use of the antibody or the antigen binding fragment according to any one of items 1 to 45 and 52 or the composition according to item 53 or 54 for an in vitro immunoassay, in particular an in vitro immunoassay for detecting α-1,6-core-fucosylated alpha-fetoprotein (AFP) or AFP-L3.
56. The use according to item 55, wherein the immunoassay is a heterogeneous immunoassay.
57. The use according to item 55, wherein the immunoassay is an immunohistochemistry (IHC) assay.
58. The use according to any one of items 55 to 57, wherein the sample for said immunoassay is a sample consisting of or being prepared from blood, plasma, or serum.
59. The use according to any one of items 55 to 58 for the detection of α-1,6-core-fucosylated AFP or a partial sequence of AFP comprising said α-1,6-core-fucosylation.
60. The use according to any one of items 55 to 59, wherein the immunoassay is an immunoassay for the detection of the glycopeptide of Formula I or a glycoprotein comprising the glycopeptide of Formula I.
61. The use of an antibody according to any one of items 55 to 60 for discriminating α-1,6-core-fucosylated AFP or a partial AFP sequence comprising said α-1,6-core-fucosylation from (i) AFP or a partial AFP sequence lacking the α-1,6-core-fucosylation and/or (ii) α-1,6-core-fucosylated proteins other than AFP.
62. An in vitro immunoassay method for detecting α-1,6-core-fucosylated AFP or a partial AFP sequence comprising said α-1,6-core-fucosylation in a sample using the antibody or antigen binding fragment as defined in any one of items 1 to 45 and 52
63. The method of item 62 comprising (i) binding the antibody or the antigen binding fragment as defined in any one of items 1 to 45 and 52 to α-1,6-core-fucosylated AFP or a partial AFP sequence comprising said α-1,6-core-fucosylation comprised in the sample so as to form a detection complex (ii) detecting the α-1,6-core-fucosylated AFP or a partial AFP sequence comprising said α-1,6-core-fucosylation by detecting the detection complex and thereby determining the presence and optionally the amount of α-1,6-core-fucosylated AFP or a partial AFP sequence comprising said α-1,6-core-fucosylation in the sample.
64. The method of item 63, wherein said method is an IHC assay, wherein the sample is a tissue slide.
65. The method of item 63, wherein the method is a body fluid immunoassay, wherein the sample is a body fluid.
66. The method of item 65, wherein the body fluid is a blood sample, seminal fluid or urine.
67. The method of item 65 or 66, wherein the body fluid is a blood sample that is whole blood, capillary blood, serum or plasma (preferably serum or plasma).
68. The method of any one of items 65 to 67, wherein the method comprises (i) pretreating the sample with a pretreatment agent and (ii) incubating the pretreated sample with the antibody or antigen binding fragment as defined in any one of items 1 to 45 and 52.
69. The method of item 68, wherein the pretreating agent is a pretreatment reagent as defined in any one of items 74 to 78.
70. The method according to any one of items 63 to 69, wherein said method discriminates α-1,6-core-fucosylated AFP or a partial AFP sequence comprising said α-1,6-core-fucosylation from AFP lacking the α-1,6-core-fucosylation or a partial sequence thereof lacking the α-1,6-core-fucosylation.
71. The method of any one of items 65 to 70, wherein the method is a sandwich immunoassay, and wherein the method comprises incubating the sample with a AFP-specific antibody or antigen binding fragment, which does not compete for binding with the antibody according to items 1 to 45 and 52 and which does bind AFP independent of the α-1,6-core-fucosylation.
72. The method of item 71, wherein the AFP-specific antibody is anti-AFP (Tu-11).
73. The method of any one of items 63 to 72, wherein the method is for aid in detecting or detecting hepatocellular carcinoma, in embodiments early hepatocellular carcinoma.
74. A pretreatment reagent for treating a sample comprising α-1,6-core-fucosylated AFP, said pretreatment reagent comprising a reducing agent, in particular a protein reducing agent.
75. The pretreating reagent of item 74, wherein the reducing agent is selected from the group consisting of: dithiothreitol (DTT), tris(2-carboxyethyl)phosphine (TCEP), β-mercaptoethanol and dithiobutylamine (DTBA).
76. The pretreating reagent of item 74 or 75, wherein the reducing agent is DTT.
77. The pretreating reagent of any one of items 74 to 76, wherein the pretreatment agent further comprises chelating agent, in embodiments a chelating agent for ions, in particular a chelating agent for divalent ions, in embodiments a chelating agent selected from: ethylenediaminetetraacetic acid (EDTA), diethylenetriaminepentaacetic acid (DTPA), porphyrins, polyamines, crown ethers, or ethylene glycol-bis(β-aminoethyl ether)-N,N,N′,N′-tetraacetic acid (EGTA).
78. The pretreating reagent of any one of items 74 to 77, wherein the pretreating reagent further comprises a buffer, such as Tris.
79. A kit comprising the antibody or antigen binding fragment as defined in any one of items 1 to 45 and 52.
80. The kit according to item 79 that is an immunoassay kit.
81. The kit according to item 79 or 80, wherein the kit is for detecting or quantifying α-1,6-core-fucosylated AFP or a partial AFP sequence comprising said α-1,6-core-fucosylation.
82. The kit according to any one of items 79 to 81, wherein the kit comprises a pretreatment agent such as a pretreatment agent as defined in any one of items 74 to 78.
83. The kit according to item 82, wherein the antibody or antigen binding fragment and the pretreatment reagent are provided in separate containers.

The following definitions and embodiments apply to all aspects and embodiments of the invention provided herein above and in the claims.

As is known in the art, human AFP is a glycoprotein having the amino acid sequence SEQ ID NO: 1 (or natural occurring variants thereof as described in Uniprot ID P02771) comprising a single N-glycosylation site corresponding to Asn-251 of Uniprot ID P02771 (version 209). The N-glycan at Asn-251 can comprise a core fucose residue (see FIG. 1). As further understood in the art, the term “core fucosylation” within the glycan indicates that a fucose residue is α-1,6-linked to the core GlcNac residue attached to the Asn-251 of the AFP protein or partial sequence thereof comprising an Asn corresponding to Asn-251 (see FIG. 1). The terms “core-fucosylation” and “α-1,6-core-fucosylation” are recognized to be interchangeable. Accordingly, the terms “specific for (or specifically binding to) core-fucosylated AFP and/or partial sequences of AFP comprising the core-fucosylation” are interchangeable with the terms “specific for (or specifically binding to) α-1,6-core-fucosylated AFP and partial sequences thereof comprising the α-1,6-core-fucosylation”. The antibodies and antibody antigen binding fragments of the invention are also interchangeably referenced herein as 1,6fucAFP antibodies and antigen binding fragments thereof.

Lectins can be used in the analysis of glycoproteins. By using the selective binding capacity of a lectin to the sugar chain structure of a glycoprotein it is possible to separate and concentrate the marker glycoprotein fraction(s) having a specific sugar chain structure. In the case of AFP, the lectin derived from Lens culinaris agglutinin-A (LCA) has been widely used. Using LCA, AFP can be fractionated into the three variants L1, L2 and L3, wherein AFP-L3 has the highest affinity to LCA. The AFP-L3 fraction is composed of AFP, which is N-glycosylated at Asn-251 with an N-glycan comprising α-1,6-core-fucosylation (i.e., AFP having a fucose sugar bound to N-acetylglucosamine (GlcNAc), which is located at a reducing terminal of an N-type sugar chain via an α-1,6 bond). Accordingly, as AFP-L3 is composed of α-1,6-core-fucosylated AFP, the terms AFP-L3 and α-1,6-core-fucosylated AFP are used herein interchangeably. The Lens culinaris agglutinin (LCA)-reactive fraction of α-fetoprotein (AFP-L3) is specifically increased in patients with HCC (Khien V V et al., The International Journal of Biological Markers. 2001; 16(2):105-111).

The terms “antibody”, “antibodies”, and analogous terms as used herein relate to full immunoglobulin molecules and encompass naturally-occurring forms of antibodies (including but not limited to IgG, IgA, IgM, IgE) as well as recombinant antibody constructs including but not limited to single-chain antibodies, chimeric antibodies, humanized antibodies, antibody-fusion proteins, multi-specific antibodies, and multivalent antibodies; as well as antigen binding fragments and derivatives of all of the foregoing. The terms “antibody”, “antibodies”, and analogous terms as used herein also refer to antigen binding fragments thereof if these are not explicitly also mentioned. Antigen binding fragments of antibodies may be referenced herein as antibody antigen binding fragment, and/or simply antigen binding fragment. These terms refer to one or more fragments of an antibody that retain the ability to specifically bind to the target antigen, i.e. α-1,6-core-fucosylated alpha-fetoprotein (AFP) or a partial sequence of AFP comprising said α-1,6-core-fucosylation (e.g. glycopeptide of Formula I or Formula II), as known in the art, including but not limited to antigen binding fragments comprising an Fv domain (i.e., paired heavy and light chain variable domains), such as Fab, Fab′, F(ab′)2, and Fv fragments as well as recombinant constructs such as single-chain Fv domains, known in the art as scFvs. The terms also includes antibody antigen binding fragments that comprise a single, unpaired heavy or light chain variable domain as known in the art that retains the ability to specifically and selectively bind antigen as defined herein, including but not limited to single domain antibodies (also referenced in the art as sdAbs, dAbs, and/or nanobodies) and VHH domains based on the heavy chains of camelids.

In certain embodiments the monoclonal antibodies of the invention may be a full-immunoglobulin, Fab, Fab′, F(ab′)2, Fv or scFv. In a specific embodiment, the monoclonal antibody of the invention may be a Fab fragment.

In certain embodiments, the monoclonal antibodies of the invention may be multivalent antibodies.

“Multivalent antibody” as used herein relates to antibodies comprising at least three Fv or Fab domains (e.g. 2, 3, 4, 5, 6, 7, 8, 9 or 10 copies). In preferred embodiments, multivalent antibodies comprise the same Fv in at least 3, at least 4, at least 5, at least 6, at least 7, at least 8 or at least 10 copies (e.g. 2, 3, 4, 5, 6, 7, 8, 9 or 10 copies).

Exemplary but non-limiting embodiments for multivalent antibodies and methods for generating such antibodies are disclosed in WO2019/057816, which is herein incorporated by reference in its entirety. Specifically, all embodiments relating to structural configurations of such multivalent antibodies and the methods for generating such multivalent antibodies (also referred to as p3, p4, p5, p6, p7 or p8) are disclosed herein by reference.

In embodiments, a multivalent antibody of the invention may comprise a heavy chain comprising a plurality of (e.g. 2, 3, 4, 5, 6, 7, 8, 9 or 10 copies, in a particular embodiment 8) VH-CH1 domains (e.g. all comprising a (preferably the same) VH of an 1,6fucAFP antibody or antigen binding fragment of the invention), e.g. flanked by linker sequences (e.g. one of the linker sequences as described in WO2019/057816, which are incorporated herein by reference). The additional VH-CH1 domains compared to a conventional antibody may be placed upstream and/or downstream of the Hinge-CH2-CH3 sequences. The light chains in such multimeric antibodies may be conventional light chains consisting of a VL and a constant domain.

Alternative methods for generating multivalent antibodies involve the chemical polymerization/crosslinking of antibodies or antigen binding fragments.

Antibodies may be polyclonal or monoclonal. The antibodies of the invention are monoclonal. The term “monoclonal” as used herein with reference to an antibody or antigen binding fragment thereof, refers to a population of antibody polypeptides or fragments thereof produced from a single B cell clone, which population contains only one specificity of an antigen binding site capable of immunoreacting with a particular epitope of an antigen. This is in contrast with “polyclonal” antibodies and compositions, which term(s) refer to a population of antibody polypeptides or antigen binding fragments that contain multiple specificities of antigen binding sites. Also included are modified forms of monoclonal antibodies of the invention such as humanized or chimeric versions thereof, as well as recombinant antibody constructs, such as antibody (or antigen binding fragment)-fusion proteins, wherein the antibody or antigen binding fragment comprises (an) additional domain(s), e.g. for the isolation and/or preparation of recombinantly produced antibody/fragment/constructs.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs) such as complementary determining regions (CDRs). (See, e.g., Kindt et al. Kuby Immunology, 6th ed., W.H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

The term “hypervariable region” or “HVR” as used herein refers to each of the regions of an antibody variable domain which are hypervariable in sequence and which determine antigen binding specificity, for example “complementarity determining regions” (“CDRs”).

Generally, antibodies comprise six CDRs: three in the VH (CDR-H1, CDR-H2, CDR-H3), and three in the VL (CDR-L1, CDR-L2, CDR-L3). Exemplary CDRs herein include:
(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));
(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, MD (1991)); and
(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)).

Unless otherwise indicated, the CDRs are determined according to Kabat et al., supra. One of skill in the art will understand that the CDR designations can also be determined according to Chothia, supra, McCallum, supra, or any other scientifically accepted nomenclature system.

“Framework” or “FR” refers to variable domain residues other than complementary determining regions (CDRs). The FR of a variable domain generally consists of four FR domains: FR1, FR2, FR3, and FR4. Accordingly, the CDR and FR sequences generally appear in the following sequence in VH (or VL): FR1-CDR-H1(CDR-L1)-FR2-CDR-H2(CDR-L2)-FR3-CDR-H3(CDR-L3)-FR4.

As used herein, the phrase “specifically binds” in the context of an antibody or antibody antigen binding fragment indicates that the respective antigen is bound to the antibody or antibody antigen binding fragment via an antigen-antibody reaction. The term “specifically binding” also expresses that the antibody or antigen binding fragment binds to the indicated structure preferentially over other structures that may show cross reactivity. As has also been explained herein, the term discriminates from/over indicates that the antibodies and antigen binding fragments of the invention specifically bind to the target antigen (i.e. core-fucosylated AFP and/or core-fucosylated partial sequences thereof, most preferably the glycopeptides of Formula I or II), but do not specifically bind to AFP/AFP partial sequences lacking the core-fucose residue, and/or a core-fucosylated glycan in another context such as a single core-fucosylated asparagine as set out in Formula IV.

As used herein, the term “discriminate(s) from/over” and analogous terms with respect to two antigens, e.g. the antibody discriminates antigen X from/over antigen Y, indicates that the antibody or antigen binding fragment specifically binds to a target antigen X but does not specifically bind to the non-target antigen Y. Accordingly, the terms “discriminate(s)” and analogous terms as used herein means that the antibody or antigen binding fragment “does not specifically bind”/“does not significantly bind” (which are used interchangeably) to the non-target antigen. It is well known in the art that the terms “specifically bind” and “does not significantly bind” designate the degree to which an antibody discriminates between two antigens. This is because it is known that no antibody has absolute specificity, in the sense that it will react with only one epitope whatever the conditions. That is, where other (non-target) antigens are present, an antibody or antigen binding domain can react to some extent with similar epitopes on these other (non-target) antigens. However, the affinity of a monoclonal antibody or monoclonal antigen binding fragment for its target epitope/antigen is significantly greater than its affinity for related epitopes. This difference in affinity is used to establish assay conditions, under which an antibody or antigen binding fragment binds almost exclusively to a specific epitope. In this respect, the binding (or non-binding) of an antibody or antigen binding fragment to an antigen are not understood as absolutes. That is, the 1,6fucAFP antibodies and/or antigen binding fragments may exhibit some (residual) binding activity for other (non-)targets, but at significantly reduced levels relative to the binding activity for core-fucosylated AFP or core-fucosylated partial sequences of AFP, preferably the glycopeptide of Formula I or a glycoprotein comprising the glycopeptide of Formula I.

For example, the feature of discriminating a target antigen from/over a non-target antigen may be characterized by the 1,6fucAFP antibody or antibody antigen binding fragment having an affinity for the target antigen that is at least 10 fold, at least 20 fold, at least 50 fold or at least 100 fold, at least 150 fold, at least 180 fold or at least 245 fold better than the affinity for the non-target antigen (i.e. the K_{D} for the binding to the target antigen is at least 10 fold, at least 20 fold, at least 50 fold or at least 100 fold, at least 150 fold, at least 180 fold or at least 245 fold lower than for the binding to the non-target antigen). The formulation at least “XX” also covers the embodiment, that the K_{D} for the non-target antigen is so high that it can not be detected with the method used. Accordingly, whether an antibody or antigen binding fragment can discriminate the target structure from a non-target structure may be determining the K_{D} values for the respective bindings using the same method (e.g the preferred method for determining the K_{D} as described herein below.

In embodiments, the capability of an antibody to discriminate between the target antigen from/over a non-target antigen can be assessed using an immunoassay in which the binding of the to be tested antibody or antigen-binding fragment to a target structure is detected. Using such immunoassay the immunoassay signal obtained with a first sample comprising the target antigen in a defined concentration can be compared with the immunoassay signal obtained with a second sample comprising the non-target antigen in the same concentration. An immunoassay signal of the first sample being higher than the immunoassay signal from the second sample indicates a discrimination between the target antigen and non-target antigen. In embodiments, the tested antibody or antigen-binding fragment may discriminate between the target structure and the non-target structure if the immunoassay signal of the first sample is at least 5-fold, at least 10 fold, at least 20 fold, at least 40 fold, at least 50 fold, or at least 100 higher than for the second sample. An exemplary but non-limiting immunoassay that can be used for such analysis is provided in Example 7. An exemplary concentration of the target antigen and non-target antigen may be 12 nm.

The term “substitution”, “exchange” or “mutate” as used herein in the context of amino acids refers to the replacement of an amino acid with another amino acid. The deletion of an amino acid at a certain position and the insertion of one (or more) amino acid(s) at a different position is explicitly not encompassed by the term “substitution”. As noted, the present invention encompasses conservative or highly conservative amino acid substitutions as have been defined herein above.

Amino acids are herein either spelled out or abbreviated using a 1-letter code or a three letter code.

In the context of the invention, it is referred to variants of sequences (in particular CDRs). These variants typically comprise one or more amino acid substitutions. It is evident that the variant CDRs are functional variants, i.e. having amino acid sequences that may differ from the reference amino acid sequence but which differing sequence exhibits or maintains the same functional activity as the reference sequence in the context of the described heavy and/or light chain variable domain. Specifically, as used herein, the term same functional activity means that the antibody or antibody binding fragment of the invention comprising one or more variant CDRs will maintain the specific binding to α-1,6-core-fucosylated alpha-fetoprotein (AFP) or a partial sequence of AFP comprising said α-1,6-core-fucosylation (e.g. glycopeptide of Formula I or Formula II) and its property to discriminate these structures from non-α-1,6-core-fucosylated AFP or partial sequences (e.g. glycopeptide of formula III or peptide of SEQ ID NO: 2 or 25) and/or the glycan of formula IV. In embodiments, functional activity may also mean that the herein referred to kinetic parameters for the binding to the glycopeptide of formula I are preserved.

As used in the context of the invention, a “conservative amino acid substitution” means the substitution of an amino acid with another amino acid selected from its same physicochemical group, wherein the physicochemical groups of amino acids are
a) the nonpolar, hydrophobic amino acids consisting of Gly, Ala, Val, Leu, Ile, Phe, Tyr, Trp, and Met;
b) the polar, neutral amino acids consisting of Ser, Thr, Asn, and Gln;
c) the positively charged, basic amino acids consisting of Arg, Lys, and His, and
d) the negatively charged, acidic amino acids consisting of Asp and Glu,
   wherein if Cys is to be conservatively substituted, it is substituted with Ser or Ala, and wherein if Pro is to be conservatively substituted it is substituted with Ala.

As used in the context of the invention, a “highly conservative amino acid substitution” means the following amino acid substitutions:
a) substitution of Ala with Val, Leu, Ile or Gly;
b) substitution of Arg with Lys;
c) substitution of Asn with Gln;
d) substitution of Asp with Glu;
e) substitution of Cys with Ser;
f) substitution of Gln with Asn;
g) substitution of Glu with Asp;
h) substitution of Gly with Ala;
i) substitution of His with Arg;
j) substitution of Ile with Leu, Val or Ala;
k) substitution of Leu with Ile, Val or Ala;
l) substitution of Lys with Arg;
m) substitution of Met with Leu, Ile or Val;
n) substitution of Phe with Tyr or Trp;
o) substitution of Pro with Ala;
p) substitution of Ser with Thr;
q) substitution of Thr with Ser;
r) substitution of Trp with Phe or Tyr;
s) substitution of Tyr with Phe or Trp; and
t) substitution of Val with Leu, Ile or Ala.

As used herein, the term “% sequence identity” in connection with amino acid sequences of polypeptides/peptides and/or nucleic acid sequences or nucleic acid molecules describes the number of matches of identical amino acid or nucleic acid residues of two or more aligned sequences as compared to the number of residues making up the overall length of the compared sequences (or the overall compared portions thereof). Using an alignment of two or more sequences or subsequences, the percentage of residues that are the same may be determined when the (sub)sequences are compared and aligned for maximum correspondence over a window of comparison, or over a designated region as measured using a sequence comparison algorithm as known in the art, or when manually aligned and visually inspected. Non-limiting examples of algorithms for use in determining sequence identity include, for example, those based on the NCBI BLAST algorithm (Altschul et al., Nucleic Acids Res 25(1997), 3389-3402), CLUSTALW computer program (Thompson, Nucl. Acids Res. 2(1994), 4673-4680) or FASTA (Pearson and Lipman, Proc. Natl. Acad. Sci., 85(1988), 2444). Although the FASTA algorithm typically does not consider internal non-matching deletions or additions in sequences, i.e. gaps, in its calculation, this can be corrected manually to avoid an overestimation of the % sequence identity. CLUSTALW, however, does take sequence gaps into account in its identity calculations. Also available are the BLAST and BLAST 2.0 algorithms (Altschul et al., Nucl Acids Res., 25(1977), 3389).

In the context of the invention glycopeptides of AFP (e.g. Formula I, Formula II, Formula III) and N-glycan structures (e.g. Formula IV) are disclosed. In the respective formula described herein above the type of the linkages is not show. Preferred linkages are the ones indicated in the exemplary glycan structure shown in FIG. 1.

Accordingly, in specific embodiments, the glycopeptide of Formula I may have the following structure Ia

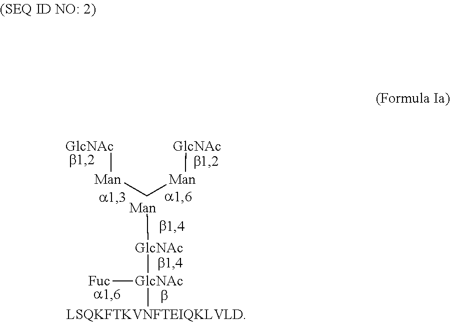

Accordingly, in specific embodiments, the glycopeptide of Formula II may have the following structure IIa

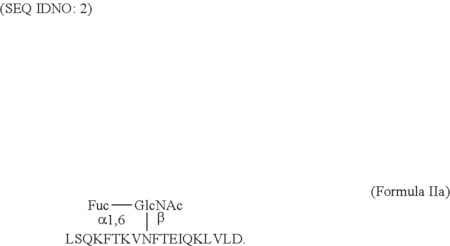

Accordingly, in specific embodiments, the glycopeptide of Formula III may have the following structure IIIa

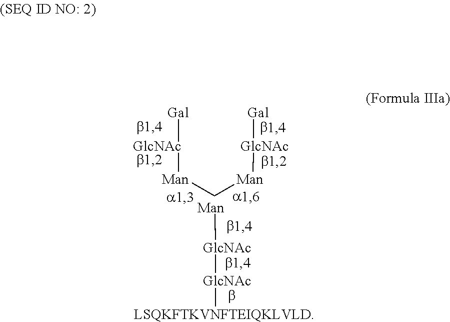

Accordingly, in specific embodiments, the N-glycan of Formula IV may have the following structure IVa

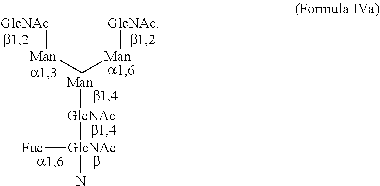

As used herein, “nucleic acid molecule”, “nucleic acid sequence”, “polynucleotide” and analogous terms include both genomic DNA and cDNA, as well as RNA capable of driving expression of an antibody or antigen binding fragment of the invention. It is understood that the term “RNA” as used herein comprises all forms of RNA including mRNA, tRNA and rRNA but also genomic RNA, such as in case of RNA of RNA viruses. Preferably, embodiments reciting “RNA” are directed to mRNA. The nucleic acid molecules/nucleic acid sequences of the invention may be of natural as well as of synthetic or semi-synthetic origin. In embodiments, the nucleic acids/nucleic acid sequences of the invention may be isolated. Thus, the nucleic acid molecules may, for example, be nucleic acid molecules that have been synthesized according to conventional protocols of organic chemistry, according to recombinant methods, or produced semi-synthetically, e.g. by combining chemical synthesis and recombinant methods. The person skilled in the art is familiar with the preparation and the use of such nucleic acid molecules.

“Immunoassays” as used herein are well-established bioanalytical methods in which detection or quantification of an analyte depends on the reaction of the analyte and at least one analyte-specific binding agent, thus forming an analyte:binding agent complex (also referred to as detection complex). In the context of the present invention, at least one of the at least one analyte specific binding agent is an antibody of the invention. The specific embodiment of a “sandwich” immunoassay can be used for analytes possessing more than one recognition epitopes. Thus, a sandwich assay requires at least two binding agents that attach to non-overlapping epitopes on the analyte. In a “heterogeneous sandwich immunoassay” one of the binding agents has the functional role of an analyte-specific capture binding agent; this binding agent is or (during the course of the assay) becomes immobilized on a solid phase. A second analyte-specific binding agent is supplied in dissolved form in the liquid phase. A sandwich-like complex is formed once the respective analyte is bound by a first and a second binding agent (binding agent-1:analyte:binding agent-2). The sandwich-like complex is also referred to as “detection complex”. Within the detection complex the analyte is sandwiched between the binding agents, i.e. in such a complex the analyte represents a connecting element between the first binding agent and a second binding agent.

The term “heterogeneous” (as opposed to “homogeneous”) denotes two essential and separate steps in the assay procedure. In the first step, a detection complex containing label is formed and immobilized, however with unbound label still surrounding the complexes. Prior to determination of a label-dependent signal unbound label is removed from the immobilized detection complex, thus representing the second step. In contrast, a homogeneous assay produces an analyte-dependent detectable signal by way of single-step incubation and does not require a washing step.

In a heterogeneous immunoassay the solid phase is functionalized such that it may have bound to its surface the functional capture binding agent (the first binding agent), prior to being contacted with the analyte; or the surface of the solid phase is functionalized in order to be capable of anchoring a first binding agent, after it has reacted with the analyte. In the latter case, the anchoring process must not interfere with the binding agent's ability to specifically capture and bind the analyte. A second binding agent present in the liquid phase is used for detection of bound analyte. Thus, in a heterogeneous immunoassay the analyte is allowed to bind to the first (capture) and second (detector) binding agents. Thereby a “detection complex” is formed wherein the analyte is sandwiched between the capture binding agent and the detector binding agent. In a typical embodiment, the detector binding agent is labeled prior to being contacted with the analyte; alternatively a label is specifically attached to the detector binding agent after analyte binding. With the detection complexes being immobilized on the solid phase, the amount of label detectable on the solid phase corresponds to the amount of sandwiched analyte. After removal of unbound label, immobilized label indicating presence and amount of analyte can be detected.

A “competitive immunoassay” as used herein preferably employs a single binding agent directly interacting with the analyte. A “competitive heterogeneous immunoassay typically detects a signal of a detection label that inversely corresponds to the amount of analyte in a sample.

“Detectable labels” as used herein relates to labels that allow for detection. According to an embodiment of the current invention a detectable label is an enzyme, or a label emitting light, in an embodiment fluorescence, luminescence, chemiluminescence, electro-chemiluminescence or radioactivity. In a preferred embodiment the label is an electrochemiluminescent label, in an embodiment Tris(2,2′-bipyridyl)ruthenium(II)-complex (Ru(bpy)). As the interference is caused by the three-dimensional structure of the label molecule that attracts auto-antibodies and similar interfering molecules and not by the signal-emitting mechanism of said label, such as e.g. light or radioactivity, all the above-referenced labels can be used in the current invention.

“Capture labels” as used herein relates to labels that can immobilize a capture agent (e.g. an antibody having a capture label attached thereto) on a surface (e.g., on a magnetic particle such as a microbead). Non-limiting examples are members of binding pairs. A non-limiting example for a capture label is biotin or derivatives thereof, which can interact with streptavidin or derivatives thereof. Different capture labels are well known in the art.

The term “and/of” should be understood to mean either one, or both of the alternatives.

As used herein and unless stated otherwise, it is to be understood that the term “about” is used synonymously with the term “approximately”. Illustratively and unless stated otherwise, the use of the term “about” when used in conjunction with a stated numerical value or range denotes somewhat more or somewhat less than the stated value or range, to within a range of ±15% of that stated, ±10% of that stated, ±5% of that stated, or conveniently ±2% of that stated. Such values are thus encompassed by the scope of the claims reciting the terms “about” or “approximately”.

The term “biomarker” or “marker” as used herein refers generally to a molecule, including a gene, protein, carbohydrate structure, or glycolipid, metabolite, mRNA, miRNA, protein, DNA (cDNA or genomic DNA), DNA copy number, or an epigenetic change, e.g., increased, decreased, or altered DNA methylation (e.g., cytosine methylation, or CpG methylation, non-CpG methylations); histone modification (e.g., (de)acetylation, (de) methylation, (de) phosphorylation, ubiquitination, SUMOylation, ADP-ribosylation); altered nucleosome positioning, the expression or presence of which in or on a mammalian tissue or cell can be detected by standard methods (or methods disclosed herein) and which may be predictive, diagnostic and/or prognostic for an individual's health or a disease. Therefore sometimes herein below the more general term “marker” is also used while discussing the more general terms and definitions. The term marker also includes a glycan structure or glycan, or a glycopeptide as analyzed in the present disclosure.

The term “in vitro method” is used to indicate that the method is performed outside a living organism and preferably on body fluids, isolated tissues, organs or cells. An in vitro method may also be referred to an ex vivo method.

Hepatocellular carcinoma (HCC) is the major histologic type among primary liver cancers occurring worldwide, accounting for 70% to 85% of the total burden. It is known, that underlying liver diseases such as liver fibrosis and cirrhosis are the main risk factors for the development of HCC. HCC can be treated by resection, liver transplantation, or local ablation with radiofrequency for patients diagnosed at an early stage. The 5-year survival rate of the HCC patients may be as high as 70% if this malignancy is diagnosed in an early stage. However, the 5-year survival rate of the HCC patients decreases significantly the later the disease is diagnosed and drops to only 15%, if HCC is diagnosed in the late stage of disease (Tsuchiya N, Sawada Y, Endo I, et al. Biomarkers for the early diagnosis of hepatocellular carcinoma. World J Gastroenterol. 2015; 21(37):10573-83; Siegel R, Naishadham D, Jemal A. Cancer statistics, 2013. CA: A Cancer Journal for Clinicians. 2013; 63(1):11-30).

The term “aiding in the detection of hepatocellular carcinoma (HCC)” is used to indicate that the method according to the present invention will help/aid a medical professional including, e.g., a physician in assessing whether an individual has HCC or is at risk of developing HCC. As will be appreciated several alternative methods (e.g. ultra sound, Radiography, MRT, or CT) can be used by a physician and combined with in vitro biomarker data, like glycan structure data, to detect or to exclude presence of HCC. The final diagnosis of HCC is usually made from a tissue biopsy or from a tissue sample after surgery. The term “aiding in the detection of HCC” includes that the method is used as sole diagnostic utility or is used as one of multiple diagnostic utilities. Not in all (100%) of the patients with HCC the amount of the marker is above the reference level and not in all healthy individuals the level of the marker is lower than the reference level or cut-off level. As the skilled artisan will appreciate, in many diseases, no biochemical marker has 100% specificity and at the same time 100% sensitivity. Rather the marker analyzed or a marker combination comprising this marker gives a certain likelihood, e.g. at a given level of specificity or at a given level of sensitivity that an individual whose sample has been analyzed has a certain clinical status, e.g. has HCC. The skilled artisan is fully familiar with the mathematical/statistical methods used to calculate specificity, sensitivity, positive predictive value, negative predictive value, reference value or total error. Any of these parameters can be calculated and used to obtain an indication of the presence or absence of HCC.

One convenient goal to quantify the diagnostic accuracy of a laboratory test is to express its performance by a single number. The most common global measure is the area under the curve (AUC) of the ROC plot. The area under the ROC curve is a measure of the probability that the perceived measurement will allow correct identification of a condition (or the differentiation of one condition from the other). Values typically range between 1.0 (perfect separation of the test values of the two groups) and 0.5 (no apparent distributional difference between the two groups of test values). The area does not depend only on a particular portion of the plot such as e.g. the point closest to the diagonal or the sensitivity at 90% specificity, but on the entire plot. This is a quantitative, descriptive expression of how close the ROC plot is to the perfect one (area=1.0). In the context of the present invention, the two different conditions can be whether a patient has HCC or does not have HCC.

The term “subject” or “individual” as used herein relates to a single person. The subject may be healthy or a patient, e.g. having cirrhosis, being at risk of developing HCC, experiencing or having experienced one or more signs, symptoms, or other indicators of HCC. Intended to be included as a subject are any subjects involved in clinical research trials not showing any clinical sign of disease, or subjects involved in epidemiological studies, or subjects whose samples may serve as controls. In embodiments, the subject may be known to be at risk for developing HCC, e.g. through chronic alcohol consumption, hepatitis B and/or hepatitis C infection, non-alcoholic fatty liver disease, Wilson's disease, hereditary hemochromatosis, alpha1-antitrypsin deficiency, primary biliary cirrhosis, autoimmune hepatitis and other risk factors. Other risk factors may include obesity and/or liver transplantation. In embodiments, the subject may be known to be at risk for developing HCC, e.g through liver fibrosis, non-cirrhotic liver diseases, NASH, chronic HBV and chronic HCV.

In one embodiment according to the present disclosure, the subject from which the sample to be investigated had been obtained is a healthy subject and is screened for (the presence of) HCC as part of routine oncology surveillance.

In one embodiment according to the present disclosure, the subject from which the sample to be investigated had been obtained is a subject at risk for developing HCC and is screened for (the presence of) HCC as part of routine oncology surveillance.

A subject may be at risk to develop HCC if the subject is known to suffer from a chronic liver disease, viral- or non-viral hepatitis and/or liver cirrhosis.

In one embodiment according to the present disclosure the subject from which the sample to be investigated had been obtained has chronic liver disease, viral- or non-viral hepatitis, liver cirrhosis and is subjected to a differential diagnosis for presence or absence of HCC.

A “sample” as used in the context of the present disclosure may be a liquid sample comprising or expected to comprise α-1,6-core-fucosylated AFP or partial sequences thereof comprising the α-1,6-core-fucosylation. The sample may in particular be a body fluid, such as, but not restricted to a blood sample, cerebrospinal fluid, seminal fluid, saliva or urine. In preferred embodiments, the sample is a blood sample, such as whole blood, serum or plasma. In even more preferred embodiments, the sample is serum or plasma.

The term “amount” or “level” of an analyte in a sample as used herein relates to any absolute measure which corresponds to the amount or concentration of said analyte in the sample or is proportional to the absolute amount or concentration of said analyte in the sample; or any relative measure, i.e. a measure representing an amount or concentration of the analyte being relative to a reference amount or concentration, respectively.

As used herein the term “reference amount” (or “reference level”) for an analyte (e.g. of a glycan structure or a glycopeptide) refers to an independently established, predetermined amount of said analyte. As the skilled artisan will appreciate the reference amount is predetermined and set to meet routine requirements in terms of e.g. specificity and/or sensitivity for the purpose of detecting HCC (e.g. early HCC). Accordingly, the reference amount may be selected such that it is indicative for HCC (e.g. early HCC). The requirements for detecting HCC can vary, e.g. from regulatory body to regulatory body. It may for example be that assay sensitivity or specificity, respectively, has to be set to certain limits, e.g. to 80%, 90%, 95% or 98%, respectively. These requirements may also be defined in terms of positive or negative predictive values. For any selected requirement e.g. in terms of level of sensitivity or specificity, respectively, the reference range (if evaluated and decreased values are indicative of an abnormal status) or the reference level or cut-off level (if either evaluated or decreased values are indicative of an abnormal status) can be determined by the skilled artisan. In analogy and according to the same principles a reference ratio can be determined.

As used herein the term “reference value”, e.g. in the context of a reference value for a score, relates to an independently established, predetermined value for the respective parameter (e.g. a score). As the skilled artisan will appreciate the reference value is predetermined and set to meet routine requirements in terms of e.g. specificity and/or sensitivity for the purpose of detecting HCC (e.g. early HCC). Accordingly, the reference value may be selected such that it is indicative for HCC (e.g. early HCC). What is said above with respect to reference amount applies mutatis mutandis.

The term “glycan structure” or “glycan” are used interchangeably herein. In the present disclosure the glycans investigated are N-glycans. Therefore the terms glycan and N-glycan herein are used interchangeably. A glycan or glycan structure consists of various types of sugar moieties. The linkages are preferably as shown in FIG. 1. N-glycan structures are attached the side chain of amino acid asparagine.

The term “glycopeptide” is used to refer to a peptide or to a peptide fragment of a larger polypeptide comprising an amino acid to which a glycan is covalently attached.

The word “comprise”, and variations such as “comprises” and “comprising”, is to be understood to imply the inclusion of a stated integer or step or group of integers or steps but not the exclusion of any other integer or step or group of integers or steps.

As used in this specification and the appended claims, the singular forms “a”, “an”, and “the” include plural referents, unless the content clearly dictates otherwise.

Concentrations, amounts, and other numerical data may be expressed or presented herein in a “range” format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “150 mg to 600 mg” should be interpreted to include not only the explicitly recited values of 150 mg to 600 mg, but to also include individual values and sub-ranges within the indicated range. Thus, included in this numerical range are individual values such as 150, 160, 170, 180, 190, . . . 580, 590, 600 mg and sub-ranges such as from 150 to 200, 150 to 250, 250 to 300, 350 to 600, etc. This same principle applies to ranges reciting only one numerical value. Furthermore, such an interpretation should apply regardless of the breadth of the range or the characteristics being described.

The term “about” when used in connection with a numerical value is meant to encompass numerical values within a range having a lower limit that is 5% smaller than the indicated numerical value and having an upper limit that is 5% larger than the indicated numerical value.

In the foregoing detailed description of the invention, a number of individual elements, characterizing features, techniques and/or steps are disclosed. It is readily recognized that each of these has benefit not only individually when considered or used alone, but also when considered and used in combination with one another. Accordingly, to avoid exceedingly repetitious and redundant passages, this description has refrained from reiterating every possible combination and permutation. Nevertheless, whether expressly recited or not, it is understood that such combinations are entirely within the scope of the presently disclosed subject matter.

All technical and scientific terms used herein, unless otherwise defined, are intended to have the same meaning as commonly understood by one of ordinary skill in the art. Reference to techniques employed herein are intended to refer to the techniques as commonly understood in the art, including variations on those techniques or substitutions of equivalent techniques that would be apparent to one of skill in the art.

All amino acid sequences provided herein are presented starting with the most N-terminal residue and ending with the most C-terminal residue, as customarily done in the art, and the one-letter or three-letter code abbreviations as used to identify amino acids throughout the present invention correspond to those commonly used for amino acids.

In this specification, a number of documents including patent applications and manufacturer's manuals are cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

Herein, the elements of the present invention are described. These elements are listed as aspects with specific embodiments, however, it should be understood that they may be combined in any manner and in any number to create additional aspects and embodiments. In particular, embodiments disclosed in context of one aspect apply mutatis mutandis to the other aspects. The various described examples and preferred embodiments should not be construed to limit the present invention to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

### Description of Sequences

The following amino acid sequences are referred to in the context of the present disclosure.

SEQ ID NO: 1: AFP sequence (see UniProt: P02771; version 209); Asn-251
which is the site for N-glycosylation is underlined
MKWVESIFLIFLLNFTESRTLHRNEYGIASILDSYQCTAEISLADLATIFFAQFVQEATY
KEVSKMVKDALTAIEKPTGDEQSSGCLENQLPAFLEELCHEKEILEKYGHSDCCSQSEEG
RHNCFLAHKKPTPASIPLFQVPEPVTSCEAYEEDRETFMNKFIYEIARRHPFLYAPTILL
WAARYDKIIPSCCKAENAVECFQTKAATVTKELRESSLLNQHACAVMKNFGTRTFQAITV
TKLSQKFTKVNFTEIQKLVLDVAHVHEHCCRGDVLDCLQDGEKIMSYICSQQDTLSNKIT
ECCKLTTLERGQCIIHAENDEKPEGLSPNLNRFLGDRDFNQFSSGEKNIFLASFVHEYSR
RHPQLAVSVILRVAKGYQELLEKCFQTENPLECQDKGEEELQKYIQESQALAKRSCGLFQ
KLGEYYLQNAFLVAYTKKAPQLTSSELMAITRKMAATAATCCQLSEDKLLACGEGAADII
IGHLCIRHEMTPVNPGVGQCCTSSYANRRPCFSSLVVDETYVPPAFSDDKFIFHKDLCQA
QGVALQTMKQEFLINLVKQKPQITEEQLEAVIADFSGLLEKCCQGQEQEVCFAEEGQKLI
SKTRAALGV
SEQ ID NO: 2 Peptide sequence of AFP (AS 243 to 261 of SEQ ID NO: 1);
Asn corresponding to Asn-251 is underlined
LSQKFTKVNFTEIQKLVLD
SEQ ID NO: 3 CDR-H1 19B12 and 3C5
TYGMG
SEQ ID NO: 4 CDR-H2 19B12
IIGDNGSTYYANWA
SEQ ID NO: 5 CDR-H2 3C5
IIDSGSTYYANWA
SEQ ID NO: 6 CDR-H3 19B12 and 3C5
DRDPSSSGYYFKM
SEQ ID NO: 7 CDR-L1 19B12
QASQSISSYLA
SEQ ID NO: 8 CDR-L1 3C5
QASQSIGSYLA
SEQ ID NO: 9 CDR-L2 19B12 and 3C5
GASNLES
SEQ ID NO: 10 CDR-L3 19B12 and 3C5
QTAFYIFSSDNA
SEQ ID NO: 11 VH sequence 19B12 (CDRs underlined)
QSVEESGGRLVAPGTPLTLTCTVSGIDLSTYGMGWVRQAPGKGLEWIGIIGDNGSTYYANWAK
GRFTISKTSTTVDLKMTSLTTEDTATYFCARDRDPSSSGYYFKMWGPGTLVTVSL
SEQ ID NO: 12 VH sequence 3C5 (CDRs underlined)
QSVEESGGRLVAPGTPLTLTCTVSGIDLSTYGMGWVRQAPGKGLEYIGIIDSGSTYYANWAKG
RFTISKTSTTVDLKMTSLTTEDTATYFCARDRDPSSSGYYFKMWGPGTLVTVSL
SEQ ID NO: 13 VL sequence 19B12 (CDRs underlined)
ALVMTQTPSSVSAAVGGTVTINCQASQSISSYLAWYQQKPGQPPKLLIFGASNLESGVPSRFK
GSGSGTEFTLTISDLECDDAATYYCQTAFYIFSSDNAFGGGTEVVVK
SEQ ID NO: 14 VL sequence 3C5 (CDRs underlined)
ALVMTQTPSSVSAAVGGTVTINCQASQSIGSYLAWYQQKPGQPPRLLIYGASNLESGVPSRFK
GSGSGTEFTLTISDLECDDAATYYCQTAFYIFSSDNAFGGGTEVVVK
SEQ ID NO: 15 FW1 of VH of 19B12 and 3C5
QSVEESGGRLVAPGTPLTLTCTVSGIDLS
SEQ ID NO: 16 FW2 of VH of 19B12
WVRQAPGKGLEWIG
SEQ ID NO: 17 FW2 of VH of 3C5
WVRQAPGKGLEYIG
SEQ ID NO: 18 FW3 of VH of 19B 12 and 3C5
KGRFTISKTSTTVDLKMTSLTTEDTATYFCAR
SEQ ID NO: 19 FW4 of VH of 19B12 and 3C5
WGPGTLVTVSL
SEQ ID NO: 20 FW1 of VL of 19B12 and 3C5
ALVMTQTPSSVSAAVGGTVTINC
SEQ ID NO: 21 FW2 of VL of 19B12
WYQQKPGQPPKLLIF
SEQ ID NO: 22 FW2 of VL of 3C5
WYQQKPGQPPRLLIY
SEQ ID NO: 23 FW3 of VL of 19B12 and 3C5
GVPSRFKGSGSGTEFTLTISDLECDDAATYYC
SEQ ID NO: 24 FW4 of VL of 19B12 and 3C5
FGGGTEVVVK
SEQ ID NO: 25 Peptide sequence of AFP (AS 248-256 of AFP as shown in
SEQ ID NO: 1); Asn corresponding to Asn-251 is underlined
TKVNFTEIQ
SEQ ID NO: 26 Peptide sequence of AFP (AS 243-256 of AFP as shown in
SEQ ID NO: 1); Asn corresponding to Asn-251 is underlined.
LSQKFTKVNFTEIQ
SEQ ID NO: 27 Exemplary linker in multivalent antibodies
GGGSGGGSGGGSGGGS

### DESCRIPTION OF THE FIGURES

The following figures are provided to aid the understanding of the present invention.

FIG. 1: Schematic drawing showing the N-glycan structure of AFP at Asn-251 (N251), highlighting the α-1,6-core-fucosylation.

FIG. 2 Exemplary antibody kinetics showing interactions versus fucosylated AFP(243-261)-G0F-Bi (dark grey) and non-fucosylated AFP(243-261)-G2-Bi (light grey) at 25° C. A) Selected antibodies with specific binding to fucosylated AFP-L3-peptides and acceptable background. B) Deselected antibodies with either no binding, complex binding behavior or no differentiation between fucosylated and non fucosylated AFP-L3-peptides (also referred to as AFP-L1).

FIG. 3: Kinetic profiles of antibodies 19B12, 3C5, 3D2, 3A3 and 6B6 binding to the various peptides. A) Antibody-interactions for fucosylated AFP(243-261)-G0F-Bi (BMO 35.000148). Shown are concentration series ranging from 3.3-90 nM with 30 nM as a duplicate (black). For antibodies 19B12 and 3C5, the Langmuir fitting model, Rmax global, RI=0 (light gray) is overlaid. B) Antibody-interactions for Asn(G0F)-Bi (BMO 35.000150). Shown are concentration series ranging from 3.3-270 nM with 30 nM as a duplicate (black). C) Antibody-interactions for non-fucosylated AFP(243-261)-G2-Bi (BMO 35.000151). Shown are concentration series ranging from 3.3-270 nM with 30 nM as a duplicate (black). For antibodies 19B12 and 3C5, the Langmuir fitting model, Rmax global, RI=0 (light gray) is overlaid. D) Antibody-interactions for AFP(243-261)-(Fuc-GlcNAc)-Bi (BMO 35.100001). Shown are concentration series ranging from 3.3-270 nM with 30 nM as a duplicate (black). For antibodies 19B12 and 3C5, the Langmuir fitting model, Rmax global, RI=0 (light gray) is overlaid.

FIG. 4: Simulation for antibody 19B12 binding non fucosylated AFP(243-261)-G2-Bi. The overlay shows 2 concentration ranges 7.3 μM-10 nM for the simulated and 1-270 nM for the experimental data set. Black lines depicted are the data for 270 nM of experimental and simulated data. The calculated affinity>620 nM.

FIG. 5: Affinity in solution data of antibodies 19B12 and 3C5 with n=2 binding AFP(243-261)-G0F resp. AFP(248-256).

FIG. 6: Comparison of the five antibodies 5 mAb<AFP-L3>rRb-xx-IgG-SulfoBPRu (xx: 6B6, 3A3, 3C5, 3D2, 19B12) for the Protein detection (total AFP, AFP-L3, AFP-L1 tested in two concentrations ca. 70 and 7 nM) in Elecsys® assay format. when using sample pretreatment 50 mM citrate, 20 mM EDTA, 110 mM DTT pH 4.9 (Control: with PBS+0.05% Tween 20).

FIG. 7: Method comparison Elecsys® AFP-L3 prototype a using 19B12-IgG (P8) vs. Wako AFP-L3 in ng/mL.

FIG. 8: Method comparison Elecsys® AFP-L3 prototype b using 3C5-IgG (P8) vs. Wako AFP-L3 in ng/mL.

FIG. 9: AFP-L3 detection with or without DDT in PT1 for low AFP-L3 concentration samples (master calibrators and native samples).

FIG. 10: AFP-L3 detection with or without DDT in PT1 for middle and high AFP-L3 concentration samples (master calibrators and native samples).

FIG. 11: ROC curve of Elecsys® AFP-L3 measured in the clinical panel.

FIG. 12: Alignment of 19B12 and 3C5 VH and VL sequences. CDR sequences according to Kabat are depicted in bold.

### EXAMPLES

The following examples are provided to aid the understanding of the present invention.

### Example 1: Synthesis of Peptides for Immunization and Screening

### Synthesis of Glycopeptides

Peptides were synthesized by means of fluorenylmethyloxycarbonyl (Fmoc) solid phase peptide synthesis on a peptide synthesizer (e.g. from Protein Technologies, Inc). For amino acid couplings 5 equivalents of each amino acid derivative were used. Amino acid derivatives were dissolved in dimethylformamide containing 1 equivalent of 1-Hydroxy-7-azabenzotriazol (HOAt). Peptides were synthesized on Sieber Amide resin. Coupling reactions were carried out for 5 minutes in dimethylformamide (DMF) with 5 equivalents 1-[Bis(dimethylamino)methylene]-1H-1,2,3-triazolo[4,5-b]pyridinium 3-oxide hexafluorophosphate (HATU) and 10 equivalents of N,N-Diisopropylethylamine (DIPEA) relative to resin loading. The Fmoc-group was cleaved for 8 minutes after each synthesis step using 20% piperidine in DMF. For the synthesis of disaccharide-containing peptides Fmoc-protected glycoamino acid building blocks of Asn were employed (Fmoc-Asn(α-L-Fuc(Ac)3(1-6)-β-D-GlcNAc(Ac)2)-OH and Fmoc-Asn(β-GlcNAc(Ac)3β(1-4)GlcNAc(Ac)2)-OH, respectively) and coupled by standard conditions described above. For peptides containing complex biantennary glycans (G0F and GF) Phenylalanine-threonine dipeptide was used as Fmoc-protected pseudoproline derivative and Asn was introduced as Fmoc-Asn(ODmap) After assembly of the peptide on the resin cleavage of Asp(ODmab) was achieved by washing of the resin with 2% hydrazine in DMF (5×5 minutes) followed by treatment with 5 mM Sodium hydroxide in water/methanol (1:1) for 1 h. Release of the peptide from the synthesis resin was achieved by incubation (10×3 min) with 1% trifluoroacetic acid in dichloromethane. The reaction solution was subsequently extracted with water and evaporated to dryness. The crude material was purified by flash chromatography. The identity of the purified material was analyzed by means of ion spray mass spectrometry.

Biantennary glycosylazides*) of G0F and G2 were reduced to the corresponding amine by adding 1,3-Propanedithiol (40 eq) and DIPEA (30 eq.) in methanol. After stirring for 4 h the sugar was precipitated by addition of cold diisopropyl ether. Sugar coupling to the peptide was achieved using 2 eq of sugar amine (G0F or G2), 2 eq of HATU, 2 eq of HOAt, 8 eq of DIPEA in DMF/DMSO (1:1) overnight. Subsequently the cleavage of the acid-labile protecting groups was achieved over 2 hours at room temperature with 9.5 ml trifluoroacetic acid, 0.25 ml triisopropylsilane, and 0.25 ml water. The reaction solution was subsequently mixed with cold diisopropyl ether to precipitate the peptide. The precipitate was filtered, washed again with cold diisopropyl ether, dissolved in a small amount of aqueous acetic acid and lyophilized. The crude material was purified by preparative reversed-phase HPLC using a gradient of acetonitrile/water containing 0.1% trifluoroacetic acid. The identity of the purified material was analyzed by means of ion spray mass spectrometry. An overview of all peptides synthesized and used for immunization and screening is shown in table 1.

*) Literature for chemical synthesis of bi-antennary fucosylated N-glycosyl azides: J. Seifert, C. Unverzagt, Tetrahedron Lett. 1997, 38, 7857-7860.

### Synthesis of Biotinylated Asn(G0F)

Asp-OBzl, Biotin-PEG12-NHS ester (1 equivalent) and trimethylamine (8 equivalents) were dissolved in DMF and stirred for 2.5 hours. The crude product was purified by preparative reversed-phase HPLC using a gradient of acetonitrile/water containing 0.1% trifluoroacetic acid.

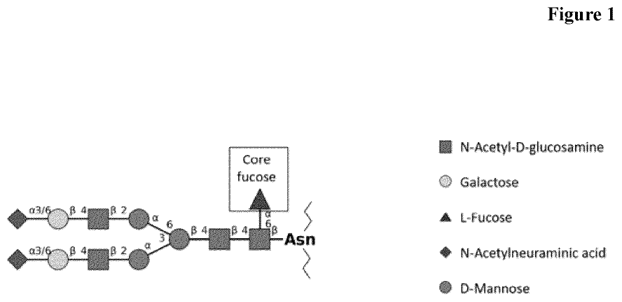

G0F-azide was reduced to the corresponding amine by adding 1,3-propanedithiol (40 eq) and DIPEA (30 eq.) in methanol. After stirring for 4 h the sugar was precipitated by addition of cold diisopropyl ether. Sugar coupling to Biotin-PEG12-Asp-OBzl was achieved using 0.5 eq of sugar amine, 1 eq of HATU, 1 eq of HOAt, 4 eq of DIPEA in DMF/DMSO (1:1) overnight. Afterwards the reaction solution was mixed with cold diisopropyl ether. The precipitate was filtered, washed again with cold diisopropyl ether, dissolved in a small amount of aqueous acetic acid and lyophilized. The crude material was purified by preparative reversed-phase HPLC using a gradient of acetonitrile/water containing 0.1% trifluoroacetic acid. The identity of the purified material was analyzed by means of ion spray mass spectrometry. A schematic drawing showing the N-glycan structure of AFP at Asn-251 (N251), highlighting the α-1,6-core-fucosylation is shown in FIG. 1.

### Immunogen Synthesis

To a solution of keyhole limpet hemocyanin (KLH) in phosphate buffer (20 mM, pH 7.2) 3-(Maleimido)propionic acid N-hydroxysuccinimide ester was added. The reaction was incubated for 5 hours at room temperature and then dialyzed against phosphate buffer (0.1 M, pH 7.0). Cysteine containing glycopeptide was dissolved in DMSO and added to a solution of maleimide-activated KLH containing 0.1 M ethylenediaminetetraacetic acid (EDTA). The solution was agitated for 5 hours at room temperature and then dialyzed against phosphate buffer (0.1 M, pH 7.0) to yield KLH-peptide conjugate.

### Glycopeptides and Peptides

TABLE 1
Overview of synthesized peptides for immunization and screening

| | | | | | SEQ |
|---|---|---|---|---|---|
| | | | | | ID |
| | | | | | NO |
| | | | | | AFP |
| Short | | BMO | | description/ | peptide |
| name | Name | number | Structure | usage | part |
| AFP(243- | AFP(243-256)[KLH- | 35.000146 | KLH-MP-C-βAla-Ahx- | short G0F peptide | 26 |
| 256)-G0F- | linker- | | βAla-βAla-LSQKFTKV- | immunogen | |
| IMG | 243; Asn(G0F)251] | | N(G0F)-FTEIQ-NH2 | | |
| AFP(243- | AFP(243-261)[Acetyl- | 35.000148 | Ac-LSQKFTKV- | biotinylated G0F | 2 |
| 261)-G0F- | 243; Asn(G0F)251; Bi- | | N(G0F)-FTEIQKLVLD- | pos. screening, | |
| Bi | PEG]: | | E(biotinyl-PEG)-NH2 | biacore | |
| AFP(243- | AFP(243-261)[Ac- | 35.000151 | Ac-LSQKFTKV-N(G2)- | biotinylated G2 | 2 |
| 261)-G2- | 243; Asn(G2)251; Bi- | | FTEIQKLVLD- | neg. screening | |
| Bi | PEG] | | E(biotinyl-PEG)-NH2 | | |
| AFP(243- | AFP(243-261)[Ac- | 35.000315 | Ac-LSQKFTKV- | G0F w/o biotin | 2 |
| 261)-G0F | 243; Asn(G0F)251]amid | | N(G0F)TEIQKLVLD- | biacore | |
| | | | NH2 | | |
| Asn(G0F)- | AFP(251)[Bi-PEG- | 35.000150 | Bi-PEG12-N(G0F)- | biotinylated | — |
| Bi | Asn(G0F)251]benzyl | | benzyl | Ans(G0F) w/o | |
| | | | | peptide | |
| | | | | biacore | |
| AFP(248- | AFP(248-256)Ac- | 35.000373 | Ac-TKVNFTEIQ-NH2 | short peptide w/o | 25 |
| 256) | 248]amid | | | sugar | |
| | | | | biacore | |
| AFP(243- | AFP(243- | 35.100001 | LSQKFTKV-N(Fuc- | biotinylated | 2 |
| 261)-(Fuc- | 261)[Asn(Fuc- | | GlcNAc- | peptide with Fuc- | |
| GlcNAc)- | GlcNAc)Bi-PEG] | | FTEIQKLVLD- | disaccharide | |
| Bi | | | E(biotinyl-PEG)-NH2 | biacore, elecsys | |
| AFP(243- | AFP(243- | 35.100002 | LSQKFTKV- | biotinylated | 26 |
| 256)- | 256)[Asn(GlcNAc- | | N(GlcNAc-GlcNAc)- | peptide with non- | |
| (GlcNAc- | GlcNAc]Bi-PEG | | FTEIQ-E(biotinyl-PEG)- | fucosylated | |
| GlcNAc)- | | | NH2 | disaccharide | |
| Bi | | | | elecsys | |

Abbreviations
Ac: N-terminal acetylation
Ahx: 6-aminohexanoic acid
βAla: beta-alanine
Bi: biotin
Fuc: L-fucoseG0F: fucosylated biantennary N-glycan
G2: non-fucosylated biantennary N-glycan
GlcNAc: N-acetylglucosamine
IMG: immunogen
MP: 3-(Maleimido)propionic acid
-NH2: C-terminal carboxyamide
PEG: polyethylene glycol
Structures of glycopeptides and peptides
BMO 35.000146: 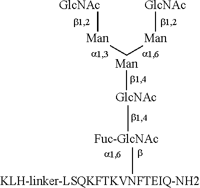
(AFP peptide part has SEQ ID NO: 26)
BMO 35.000148: 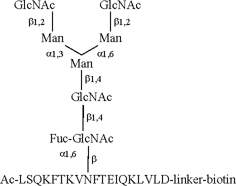
(AFP peptide part has SEQ ID NO: 2)
BMO 35.000151: 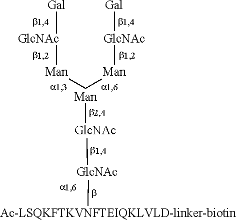
(AFP peptide part has SEQ ID NO: 2)
BMO 35.000315: 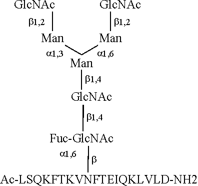
(AFP peptide part has SEQ ID NO: 2)
BMO 35.000150: 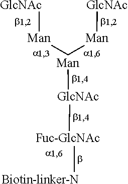
BMO 35.000373:
Ac-TKVNFTEIQ-NH2
(AFP peptide part has SEQ ID NO: 25)
BMO 35.100001: 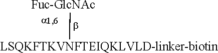
(AFP peptide part has SEQ ID NO: 2)
BMO 35.100002: 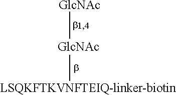
(AFP peptide part has SEQ ID NO: 26)

### Example 2: Generation of Antibodies

### Immunization

2×2 New Zealand White (NZW) rabbits, 12-16 weeks old, were immunized with the AFP-L3 glycopeptide (AFP(243-256)-G0F-IMG). To enhance the immunogenicity of the peptide, it was coupled to keyhole limpet hemocyanin (KLH) as a carrier protein. In the first month the animals were immunized weekly. Starting in the second month, the immunization schedule was reduced to once per month. For the first immunization 500 μg of KLH-coupled AFP(243-256)-G0F-IMG was dissolved in 0.9% NaCl and emulsified in 2 ml complete Freund's Adjuvant (CFA). For all following immunizations, CFA was replaced by 1 mL Incomplete Freund's Adjuvant (IFA) emulsion.

### Titer Analysis

Titer analysis was performed with an ELISA protocol. Serum titrations were performed using the biotinylated AFP(243-261)-G0F-Bi screening peptide as a positive control.

The biotinylated peptide was used for screening and was therefore immobilized on the surface of 96 well streptavidin-coated microtiter plates by incubating 100 μl per well of a 31.25 ng/ml solution for 60 min at room temperature. Subsequent washing was performed using an automated instrument (Biotek) according to the manufacturer's instructions. A small amount of serum from each rabbit (2-3 ml per animal) was collected on day 45 and day 105 after the start of the immunization campaign. The serum from each rabbit was diluted 1:300, 1:900, 1:2700, 1:8100, 1:24300, 1:72900, 1:218700 and 1:656100 with PBS containing 1% BSA. 100 μl of each dilution was added to the plate previously prepared with the screening peptides and incubated for 60 min at room temperature. Bound antibody was detected with a HRP-labeled F(ab′)2 goat anti-rabbit Fcγ (Dianova) and ABTS substrate solution (Roche). The titer of the analyzed animals was set at 50% signal decrease of the dilution curve.

TABLE 2
Exemplary titers after immunization
with the AFP-L3 peptide coupled on KLH

| Immunogen | animal number | Titer day 45 | Titer day 105 |
|---|---|---|---|
| AFP(243-261)-G0F-Bi | #K006180 | 0 | 1096 |
| AFP(243-261)-G0F-Bi | #K006181 | 0 | 0 |
| AFP(243-261)-G0F-Bi | #K006184 | 3129 | 1087 |
| AFP(243-261)-G0F-Bi | #K006185 | 0 | 561 |

As demonstrated by the results of table 2, the polyclonal sera from immunized animals bound to the AFP(243-261)-G0F-Bi screening peptide. However, the titers were extremely low, but this was expected, as a glycopeptide is an immunogen with low immunogenicity and it is extremely difficult to develop antibodies binding to such a molecule. In addition it is expected that only a very low number of antibodies will bind specifically the core-fucosylated peptide and show low cross reactivity to the non-fucosylated AFP-L1 variant.

### Enrichment and Single Cell Sorting of Antigen Reactive B-Cells

For enrichment of antigen reactive B-cells, 31.25 ng/ml biotinylated AFP(243-261)-G0F-Bi peptide was pre-incubated with the peripheral blood mononuclear cell (PBMC) pool from the immunized animals for 15 min at room temperature. After a washing step, the peptide-loaded antigen-reactive B cells were incubated with streptavidin-coated beads (Miltenyi) for 15 min at room temperature. Sorting of positive B-cells using MACS columns (Miltenyi) and subsequent incubation were performed as described in Seeber et al., PLoS One 9(2014), issue 2, e86184, with the only exception that the sorting of positive B cells involved MACS columns (Miltenyi) instead of plate binding. After a 7-day culture of B cells in 96-well plates, supernatant was collected for subsequent ELISA analysis and cells were lysed for cloning. In total, the extraction and enrichment of B-cells from immunized animals was performed 84 times, approximately 21 times per immunized animal. It is a huge advantage of the method for antibody development described here, that animals can be bleeded several times and the maturation of the antibodies can be followed over time of the immunization campaign. The large amount of sorts per animals also indicates how difficult it was to identify clones with the desired specificity for AFP-L3 with low cross reactivity to AFP-L1.

### Antibody Screening

Subsequently a Hit-ELISA (i.e. an ELISA testing the binding to the screening reagents) was used to identify B-cells expressing antibodies with desired binding characteristics, i.e. binding the AFP(243-261)-G0F-Bi peptide with the core-fucosylation, but not the glycopeptide without the core-fucose, AFP(243-261)-G2-Bi. The peptides were immobilized on the surface of streptavidin-coated 384-well plates (Nunc) by incubation of 100 μl per well of 31.25 ng/ml solutions for 60 min at room temperature, respectively. The plates were washed and 30 μl of rabbit B-cell culture supernatant was transferred to each well and incubated for 1 h at room temperature. For the detection of antibodies bound to the screening agents, HRP-labeled F(ab′)2 goat anti-rabbit Fcγ (Dianova) and ABTS substrate solution (Roche) were used according to manufacturer's instructions. Several clones were identified that bound to the glycopeptide carrying the core-fucosylation, but not to the peptide without the core-fucosylation (out of 84 B cell sorting experiments with in total 4 immunized rabbits) (Table 2). The V regions of some clones were cloned into mammalian expression vectors and subsequently expressed in 2 ml of HEK293 cells (described in Seeber et al., PLoS One 9(2014), issue 2, e86184.). After one week of expression the supernatants of the transfected HEK293 cells, containing rabbit IgG, were then used for an initial SPR Biacore based selection of a subset of antibodies fulfilling performance criteria for detailed kinetic analysis (see table 3).

TABLE 3
ELISA results of exemplary clones
producing antibody characterized by
specific binding to the positive screening
reagent but not to the negative screening

| clone | Screening peptide | OD |
|---|---|---|
| 19B12 | AFP(243-261)-G0F-Bi | 3.040 |
| | AFP(243-261)-G2-Bi | 0.279 |
| 3C5 | AFP(243-261)-G0F-Bi | 2.886 |
| | AFP(243-261)-G2-Bi | 0.936 |
| 3D2 | AFP(243-261)-G0F-Bi | 1.98 |
| | AFP(243-261)-G2-Bi | 0.1860 |
| 13G6 | AFP(243-261)-G0F-Bi | 2.475 |
| | AFP(243-261)-G2-Bi | 0.4380 |
| 3A3 | AFP(243-261)-G0F-Bi | 1.259 |
| | AFP(243-261)-G2-Bi | 0.087 |
| 6B6 | AFP(243-261)-G0F-Bi | 2.020 |
| | AFP(243-261)-G2-Bi | 0.262 |

### Example 3: Kinetic Screening and Further Kinetically Characterization

Surface Plasmon Resonance (SPR)-Spectroscopy—assisted kinetic screening and detailed kinetically characterizations were performed to select antibodies with superior kinetic profiles and target specificity.

The kinetic screening was performed at 25° C. on a GE Healthcare Biacore™ B4000 instrument. A Biacore CM5 Series S sensor was mounted into the instrument, hydrodynamically addressed and preconditioned according to the manufacturer's instructions. The system buffer was HBS ET pH 7.4, 10 mM HEPES, 150 mM NaCl, 3 mM EDTA, 0.05% (w/v) Tween20. The system buffer was supplemented with 1 mg/mL CMD (Carboxymethyldextran, Sigma, Cat.No. 86524) and was used as sample buffer for the preparation of dilution series.

A rabbit antibody capture system was immobilized on the sensor surface. A polyclonal goat anti-rabbit IgG Fc capture antibody GARbFcγ (Code-No. 111-005-046; Jackson Immuno Research, Lot #105332) was amine coupled using the EDC/NHS-chemistry according to the manufacturer's instructions.

30 μg/mL goat anti rabbit Fc gamma (GARbFcγ) in 10 mM sodium acetate buffer pH 4.5 were pre-concentrated to the spots 1, 2, 4 and 5 in the flow cells 1, 2, 3 and 4 and covalently bound to the sensor surface with densities of 12000-13000 RU. Free activated carboxyl groups were saturated with 1 M ethanolamine pH 8.5.

The spots 1 and 5 were used for the interaction measurements and spots 2 and 4 served as references. Each rabbit antibody from the primary cell supernatant was diluted 1:2 in the sample buffer and was injected at a flow rate of 10 μL/min for 2 minutes. The rabbit antibody Capture Level (CL) in resonance units (RU) was monitored. To achieve high enough sensitivity, an additional molecular mass load for the biotinylated peptides AFP(243-261)-G0F-Bi, Roche BMO 35.000148, 4.3 kDa and AFP(243-261)-G2-Bi, Roche BMO 35.000151, 4.5 kDa, was generated by Streptavidin(SA)-grafting. The biotinylated peptides, Streptavidin and Amino-PEO-Biotin (Thermo Fisher, Cat.No. 21346) were mixed with a ratio of 1:10:5 and were incubated for 2 hours at room temperature (RT). The molecular mass for the SA-grafted-AFP(243-261)-G0F-Bi and SA-grafted AFP(243-261)-G2-Bi was calculated with 64 kDa. SA-grafted peptides were injected as analytes with 150 nM to the respective surface displayed anti-AFP-L3 antibodies at 30 μL/min. The association and dissociation phases were monitored for 5 minutes.

The rabbit clones were regenerated from the sensor surface by an injection of 10 mM Glycine pH 1.5 at 20 μL/min for 20 seconds, followed by 2 injections of 10 mM Glycine pH 1.7 for 60 seconds. Kinetics were monitored by the BIAcore™ B4000 Control SW V1.1 and evaluated with the Evaluation SW V1.1. Report points Binding Late (BL), shortly before the end of the analyte injection and Stability Late (SL), shortly before the end of the dissociation phase were monitored. BL and SL data were used to characterize the antibody/antigen binding stability. Furthermore, the dissociation rate constant kₐ [s⁻¹] was calculated according to a Langmuir 1:1 model. The antigen/antibody complex stability halftime (minutes) was calculated according to the formula ln(2)/60*k_{d}. The Molar Ratio, representing the binding stoichiometry, was calculated with the formula:

MW(antibody)/MW(antigen)*BL(antigen)/CL(antibody)

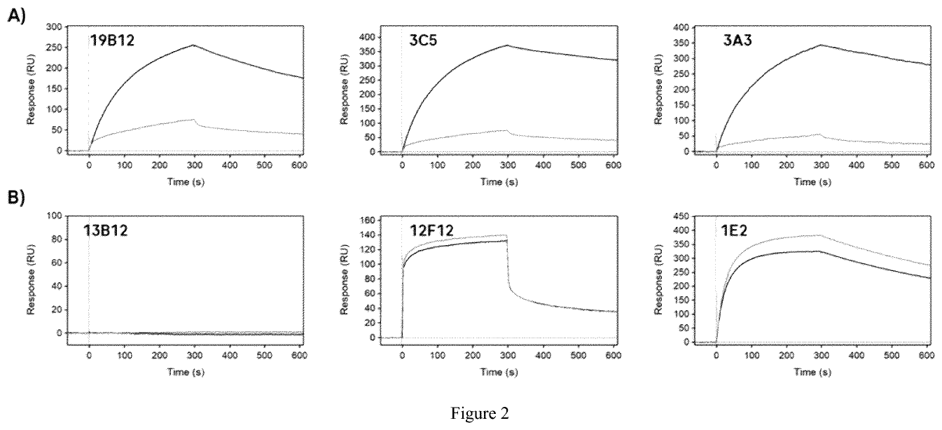

From a plurality of antibodies specific antibody kinetics were identified, where the antibodies discriminated between fucosylated and non fucosylated AFP-L3 peptides (see FIG. 2).

### Kinetic Characterization

Detailed kinetic investigations were performed using a BIAcore™ T200 instrument from GE Healthcare at 37° C. The 5 rabbit mAb<AFP-L3>L9B12, 3C5, 3D2, 3A3 and 6B6, identified via the kinetic screening, were kinetically characterized for binding AFP-L3 peptides
AFP(243-261)-G0F-Bi
Asn(G0F)-Bi
AFP(243-261)-G2-Bi
AFP(243-261)-(Fuc-GlcNAc)-Bi
   at 37° C. using a Series S C1-sensor.

The rabbit antibody capture system was immobilized on flow cells 1-4 as described with 700 to 800 RU. Flow cell 1 served as reference, flow cells 2, 3 and 4 were used for interaction measurements. 150 nM antibodies 1-5 were injected at 10 μL/min for 30 seconds. The Capture Levels (CL) in resonance units RU were monitored. Peptide analyte concentration series from 3.3-270 nM were injected at 60 μl/min. The association phase was monitored for 3 minutes, the dissociation phase was monitored for 10 minutes. The regeneration was performed by an injection of 10 mM Glycine pH 2.0 at 20 μL/min for 30 seconds, followed by 2 injections of 10 mM Glycine pH 2.25 for 60 seconds. The kinetic rate constants and the dissociation equilibrium constants K_{D} were determined using a Langmuir 1:1 fitting model according to the BIAcore™ T200 Evaluation SW 3.2. Secondly, the Langmuir 1:1 fitting Scrubber-SW V2.0c was applied.

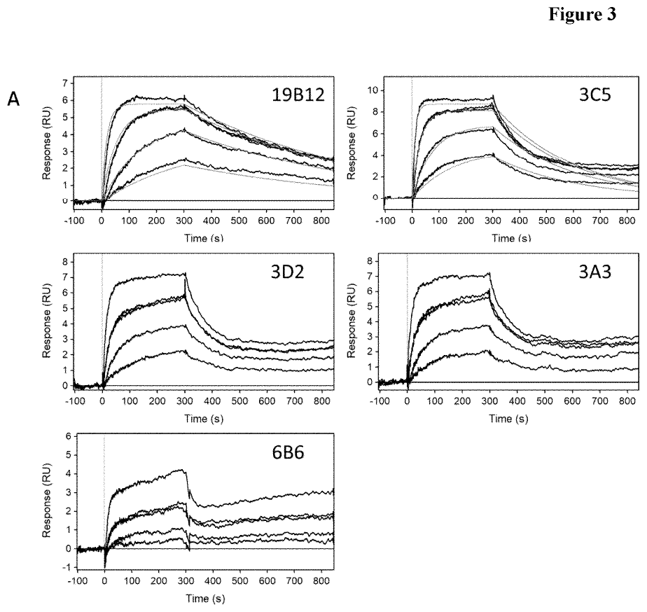

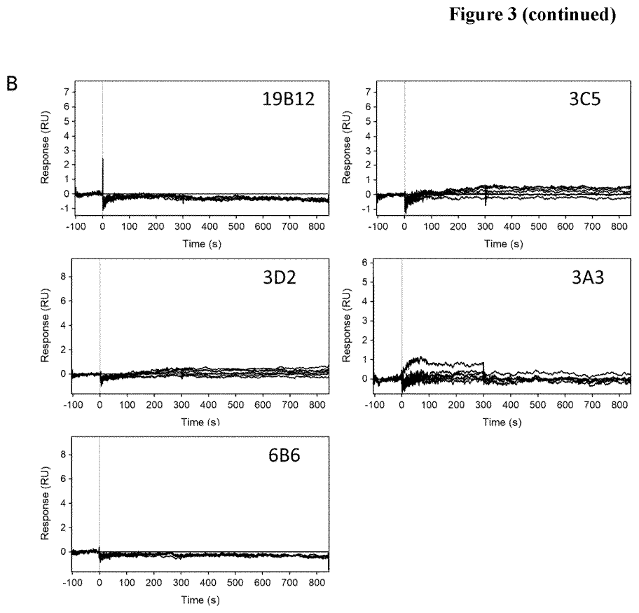

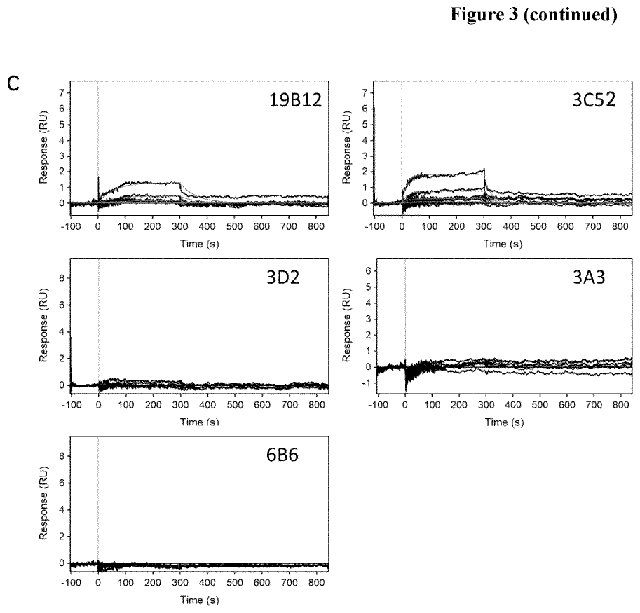

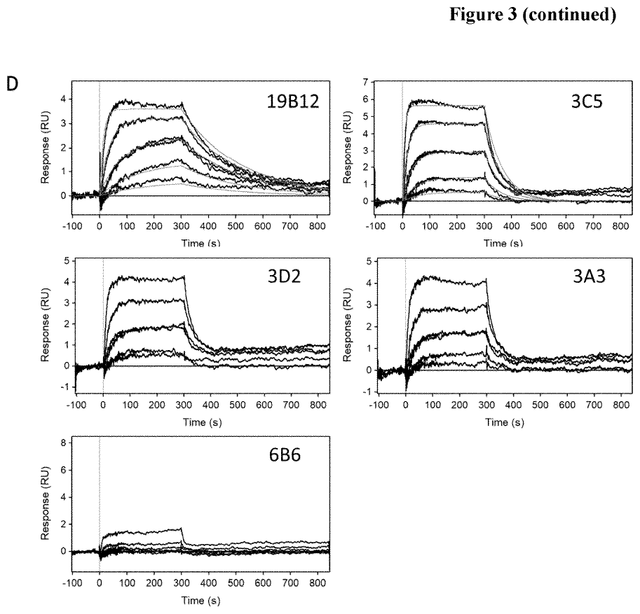

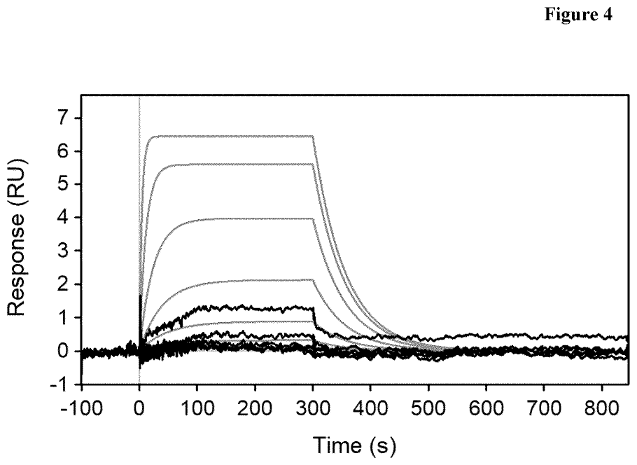

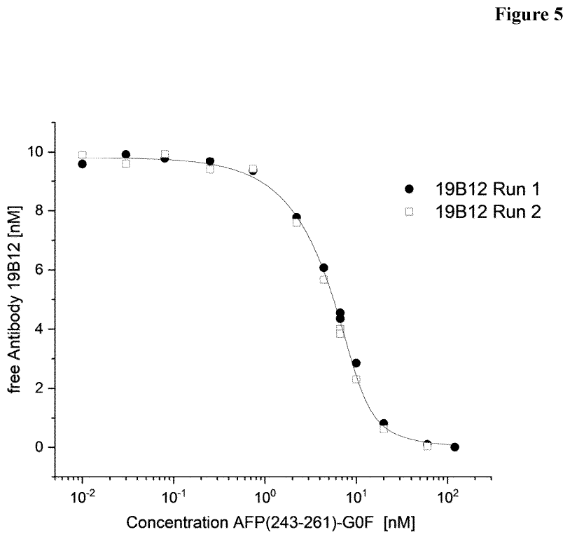

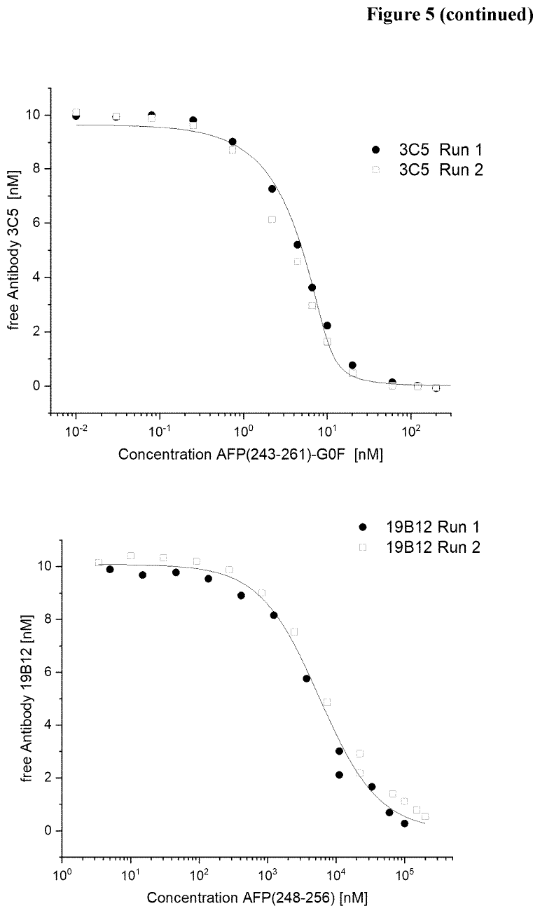

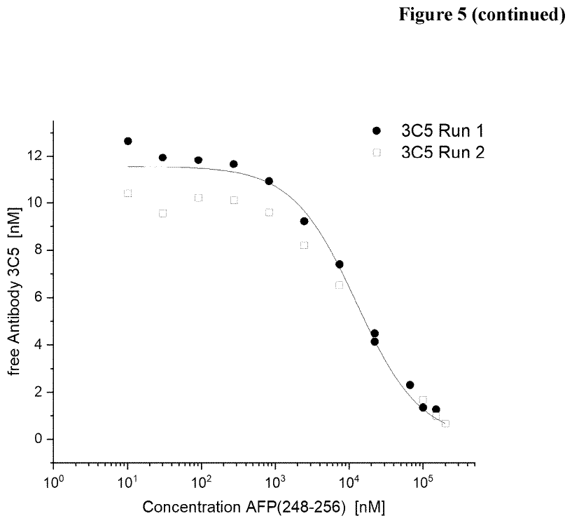

Kinetic profiles of antibodies 19B12, 3C5, 3D2, 3A3 and 6B6 binding the peptides are shown in FIG. 3. Antibody 19B12 binding kinetics versus AFP(243-261)-G0F-Bi were determined with kₐ 6.0E±05±0.07% M¹s⁻¹, kₐ 1.5E-03±0.05% s⁻¹, t_{/2 diss}=8±0.05 minutes, MR=1.4. The affinity was K_{D}=2.5±0.08% nM (see table 4 and FIG. 4). By visual inspection, the kinetic signature for antibody 3C5 is close to the antibody saturation for the highest shown concentration 90 nM. The interaction shows a complex binding behavior; therefore, stated constants represent apparent values (see table 4 and FIG. 3).

By visual inspection, the antibodies 3D2 and 3A3 show a slower complex formation, not coming close to the antibody saturation with highest concentrations, when binding the AFP(243-261)-G0F-Bi. The interactions do not obey the Langmuir law (see FIG. 3). By visual inspection, antibody 6B6 shows the weakest interaction with the fucosylated AFP(243-261)-G0F-Bi (see FIG. 3).

These analyses revealed that the clones 19B12 and 3C5 show the best performance, in terms of their specificity and affinity. Both clones are specific for the core-fucosylation and additionally bind the AFP peptide backbone. The latter is important, as a clone, only binding the core-fucose would bind to any other core-fucosylated protein in the sample, e.g. IgG type molecules.

TABLE 4
Binding constants for antibodies 19B12 and 3C5

| | | kₐ | k_{d} | t/_{2 diss} | K_{D} | Rₘₐₓ | CL | | Delta |
|---|---|---|---|---|---|---|---|---|---|
| mAb | analyte | [M⁻¹s⁻¹] | [s⁻¹] | [min] | [M] | [RU] | [RU] | MR | K_{D} |
| 19B12 | AFP(243- | 6.0E+05 ± 0.07% | 1.5E−03 ± 0.05% | 8 ± 0.05% | 2.5E−09 ± 0.08% | 5.9 | 165 | 1.4 | 1 |
| | 261)-G0F- | | | | | | | | |
| | Bi | | | | | | | | |
| | Asn(G0F)- | No detectable binding | | | | | 160 | | |
| | Bi | | | | | | | | |
| | AFP(243- | Weak binding | | | 6.2E−07 | 9.3 | 155 | | >245 |
| | 261)-G2- | detectable; simulation | | | | | | | |
| | Bi | based on data | | | | | | | |
| | AFP(243- | 2.7E+05 ± 0.1% | 4.7E−03 ± 0.06% | 2.5 ± 0.06% | 1.8E−08 ± 0.1% | 3.8 | 151 | 1.3 | >7 |
| | 261)-(Fuc- | | | | | | | | |
| | GlcNAc)- | | | | | | | | |
| | Bi | | | | | | | | |
| 3C5 | AFP(243- | 1.0E+06 ± 0.1% | 3.2E−03 ± 0.06% | 3.6 ± 0.06% | 3.1E−09 ± 0.13% | 8.8 | 216 | 1.6 | 1 |
| | 261)- | | | | | | | | |
| | G0F-Bi | | | | | | | | |
| | Asn(G0F)- | No detectable binding | | | | | 210 | | |
| | Bi | | | | | | | | |
| | AFP(243- | Weak binding | | | 5.6E−07 | | 205 | | >180 |
| | 261)-G2- | detectable | | | | | | | |
| | Bi | | | | | | | | |
| | AFP(243- | 4.5E+05 ± 0.15% | 1.6E−02 ± 0.13% | 0.7 ± 0.13% | 3.5E−08 ± 0.14% | 6.4 | 201 | 1.5 | >11 |
| | 261)-(Fuc- | | | | | | | | |
| | GlcNAc)- | | | | | | | | |
| | Bi | | | | | | | | |

The non-fucosylated AFP(243-261)-G2-Bi binding kinetics for antibody 19B12 were not determinable. A simulation for concentrations ranging between 7.3 μm-10 nM based on determined rate constants for the measured concentration range, extrapolates a weak affinity>620 nM for the non-fucosylated AFP(243-261)-G2-Bi. The theoretical response maximum Rₘₐₓ 9.3 RU for the simulated data is based on calculation of experimental capture level (CL) 155 RUT and the molecular mass of the analyte 4.3 kDa. In FIG. 4, the overlay is shown for both, simulated and experimental concentration ranges. Black lines resemble the identical concentration 270 nM of both data sets.

### Example 4: Affinity in Solution Analysis

The dissociation equilibrium constant K) was determined via the Affinity in Solution method (AiS). Following the vendor instructions for the CAP-Kit (Cytiva), peptide AFP(243-261)-G0F-Bi was captured on the CAP chip sensor surface. Mixtures of 10 nM of the antibodies 19B12 and 3C5 with varying concentrations between 120 nM and 0.01 nM of non-biotinylated AFP(243-261)-G0F resp. varying concentrations between 200 μM-0.1 nM of non-biotinylated AFP(248-256) were incubated until equilibrium was achieved. Binding events of the mixtures to the surface displayed AFP(243-261)-G0F-Bi were monitored.

With increasing peptide concentration as a competitor, the ‘free’ antibody in solution decreases. The determined free antibody concentrations for the competition experiment were plotted versus the peptide competitor concentration. The regeneration was performed using a Guanidinium/NaOH solution, as supplied by Cytiva. Two independent series were analyzed for each interaction. The Affinity in Solution model from Biacore Evaluation software was used to evaluate the data.

TABLE 5
Affinity in solution for antibodies 19B12
and 3C5 binding peptides AFP(243-261)-
G0F and AFP(248-256)

| mAb | analyte | K_{D} | Delta K_{D} |
|---|---|---|---|
| 19B12 | AFP(243-261)-G0F | 0.9 ± 0.1 nM | |
| | AFP(248-256) | 5.7 ± 0.5 μM | >5000 |
| 3C5 | AFP(243-261)-G0F | 0.4 ± 0.1 nM | |
| | AFP(248-256) | 12.6 ± 2.9 μM | >5000 |

AiS based K_{D} 0.9±0.1 nM, R² 0.99702 RU, Bₜₒₜ 9.8±0.1 nM for antibody 19B12 binding peptide AFP(243-261)-G0F is in the same range like the concentration dependent kinetics described earlier in the document and thus confirms the experimental approaches (see table 5). Moreover, the Affinity in Solution approach shows the excellent specificity of antibodies 19B12 and 3C5 versus the fucosylated AFP(243-261)-G0F by its complete competition.

For Antibody 3C5 the AiS was determined with K_{D} 0.4±0.1 nM, R² 0.98917 RU, Bₜₒₜ 9.7±0.1 nM (see table 5). AiS based K_{D} 5.7±0.5 μM, R² 0.98766 RU, Bₜₒₜ 10.1±0.1 nM resp. 12.6±2.9 μM, R² 0.94759 RU, Bₜₒₜ 11.5±0.4 nM demonstrate weak affinities for antibodies 19B12 and 3C5 binding to peptide AFP(248-256).

### Example 5: Cloning of Antibodies and Antibody Formats, Expression of Antibodies, Purification and Labeling of Antibodies

### Cloning and Expression of IgG-Type Antibodies as IgG(P8) Multivalent Format

Multivalent anti-AFP-L3 antibodies were cloned and expressed as described in WO 2019/057816 A1. In detail, to generate multivalent anti-AFP-L3 antibodies a plurality of VH-CH1 sequences flanked by linker sequences (e.g. (G3S)4; SEQ ID NO: 27) were added upstream and downstream of Hinge-CH2-CH3 encoding sequences, thereby generating heavy chains encoding for several VH-CH1 domains and cloned into an expression vector. This heavy chain vector is co-expressed with the light chain expression vector encoding a standard light chain consisting of a VL and a constant domain. Recombinant expression was performed transiently in human embryonic kidney (HEK) cells, or transiently or non-transiently in CHO cells. Transformed cells secreted the multivalent monospecific anti-AFP-L3 antibodies into the serum-free culture supernatant from which they were isolated. The final multivalent P8 antibody format comprises 8 distinct Fab moieties, all having the same sequence (i.e. of 19B12 or 3C5).

Purification and Conjugation with Ruthenium of Antibodies

Monoclonal multivalent anti-AFP-L3 antibodies of clone 19B12 and 3C5 were produced with sufficient yields and without significant losses during purification using protein An affinity chromatography of culture supernatant with MabSelect SuRe (Cytiva) according to supplier's instructions. Purified multivalent recombinant anti-AFP-L3 antibodies reacted with the labeling reagent tris-bipyridyl-ruthenium in its sulfonated form (also referred to as “sBPRu”) using standard NHS ester coupling chemistry. Under these conditions Ruthenium label is covalently attached to functional groups of Lysine amino acid residues in the antibody backbone chain.

Purification, Fragmentation and Conjugation with Biotin of mAbTU11 (Generation of mAB<AFP>M-TU11-F(ab′)₂-Bi

Monoclonal anti-AFP antibody clone TU11 was expressed in hybridoma cells and purified via standard ion-exchange chromatography using SP and Q resins. The resulting purified IgG was further processed via pepsin to generate F(ab′)₂-fragments. F(ab′)₂-fragments of TU11 were finally conjugated with Biotin-PEG24-NHS labels via surface exposed Lysines using standard NHS ester coupling chemistry. And F(ab′)₂-Bi-PEG24 conjugates were further purified using affinity chromatography with mSA (Streptavidin mutein) to remove unconjugated F(ab′)₂-fragment.

### Expression and Purification of Anti-AFP-L3 Antibodies

Monoclonal anti AFP-L3 antibodies were transiently expressed in HEK293F and purified via standard protein A affinity chromatography with MabSelect SuRe (Cytiva) according to supplier's instructions.

### Purification of Anti-Rabbit Fc Antibodies and Conjugation

Polyclonal antibodies directed against rabbit Fc were generated by immunization of sheep with purified rabbit Fc. Polyclonal anti rabbit Fc antibodies were further purified via positive immuadsorption with an rabbit Fc immunadsorber and labeled via surface exposed lysines with tris-bipyridyl-ruthenium in its sulfonated form (also referred to as “sBPRu”) using standard NHS ester coupling chemistry.

### Example 6: Purification of AFP-L3 and L1 and Labeling of Proteins

### Purification of AFP-L1 and AFP-L3

Purified AFP from a human hepatocarcinoma cell line was purchased either from BioRad (Cat. No. 13752600) or Scripps (Cat. No. 90492-0050). AFP was subjected to affinity-chromatography with Lens culinaris agglutinin (LCA) (Vector Laboratories, Cat. No. AL-103). AFP running on a LCA-column with running buffer (50 mM TRIS-HCl pH 7.5, 150 mM NaCl, 1 mM MgCl₂, 1 mM CaCl₂)) could be separated in LCA-bound (AFP-L3) und unbound (AFP-L1) forms of AFP. Bound AFP was eluted from the LCA column by adding a mixture of 200 mM α-methylmannoside and 200 mM α-methylglucoside to the running buffer.

### Biotinylation of AFP

Purified AFP was biotinylated with Biotin-DDS-NHS labels via surface exposed lysines using standard NHS ester coupling chemistry. Unreacted labels were subsequently removed via dialysis.

### Example 7: Comparison of the Selected Antibody Clones with Regard to Binding to AFP-L3 Analyte in an Immunological Assay (Elecsys® Assay Format)

Five different antibody clones that were selected based on the kinetic screening have been tested mAb<AFP-L3>rRb-xx-IgG (xx: 6B6, 3A3, 3C5, 3D2, 19B12) for their AFP-L3 specificity and their utility to produce signal dynamics in an Elecsys® based immunoassay on the Elecsys® instrument, Cobas® e601.

In brief, biotinylated peptides or proteins (samples) were incubated with the tested 1,6-fucAFP antibodies (6B6, 3A3, 3C5, 3D2, 19B12) and the ruthenylated anti-rabbit Fc antibodies (see Example 6) for 9 min. Subsequently, 40 μl Elecsys® beads (coated with streptavidin; “SA-Beads”) were added and the mixture was incubated for 9 min such that the detection complex formed in the first step became bound to the solid phase via interaction of biotin and streptavidin. After incubation, the reaction mixture was aspirated into the measuring cell where the Elecsys® beads are magnetically captured onto the surface of the electrode. Unbound substances are then removed. Application of a voltage to the electrode then induces electrochemiluminescent emission, which is measured by a photomultiplier.

### Testing Protocol and Sandwich Principle

1st incubation: 40 μl sample+60 μl Reagent 1+60 μl Reagent 2, 9 min incubation
2nd incubation: +40 μl SA-Beads. After addition of streptavidin-coated microparticles, the complex becomes bound to the solid phase via interaction of biotin and streptavidin, 9 min incubation

After incubation, the reaction mixture was aspirated into the measuring cell where the microparticles are magnetically captured onto the surface of the electrode. Unbound substances are then removed. Application of a voltage to the electrode then induces electrochemiluminescent emission which is measured by a photomultiplier.
Reagent 1: mAb<AFP-L3>rRb-xx-IgG (1 μg/mL) (xx: 6B6, 3A3, 3C5, 3D2, 19B12)
Reagent 2: pAb<rRb-Fcg>-S-IgG(IS)-sulfoRu(Sux) (0.5 μg/mL)
Test buffer: 40 mM NaPi pH7.5/150 mM NaCl/0.1% MIT/0.1% Oxypyrion/0.1% Polidocanol/0.1% PAK—R-IgG/2% RPLA 4

### Samples:

The following biotinylated peptides diluted in PBS/0.05% Tween-20 (2 concentration levels) were tested:
1. AFP-(243-261)-G0F-Bi: with the core-fucosylation
2. AFP-(243-261)-G2-Bi: without core-fucose (AFP-L1 variant)
3. AFP-(243-261)-(Fuc-GlcNAc)-Bi Disaccharide L3
4. AFP(243-256)-(GlcNAc-GlcNAc)-Bi: Disaccharide L1

Additionally, the following biotinylated proteins were also tested:
5. total α-fetoprotein (AFP), containing AFP-L3 fraction, derived from a human hepatoma cell line

Lens culinaris agglutinin A (LCA) affinity chromatography was performed with AFP total.

Two fractions were isolated:
a) α-fetoprotein (AFP-L3) is the fucosylated variant of AFP that reacts with Lens culinaris agglutinin A
b) α-fetoprotein (AFP-L1) is the non-fucosylated variant of AFP that should not react with Lens culinaris agglutinin A

As shown in table 6, all tested antibodies are binding the peptides AFP-(243-261)-G0F-Bi and AFP-(243-261)-(Fuc-GlcNAc)-Bi. The highest signal dynamics were observed with the clones 3C5 and 19B12, confirming the findings in the kinetic analysis suggesting these two clones as the best binders.

TABLE 6
initial Elecsys ® counts measurement comparing 5 mAB candidates
against binding motives on peptides and proteins. All five antibodies
bind to the peptides AFP-(243-261)-G0F-Bi and AFP-(243-261)-(Fuc-GlcNAc)-Bi).

| | Sample | 6B6 | 3A3 | 3C5 | 3D2 | 19B12 |
|---|---|---|---|---|---|---|
| | Conc. (nM) | Counts | | | | |
| PBS | — | 577 | 560 | 570 | 564 | 580 |
| AFP-(243-261)-G0F- | 12 | 581519 | 1091686 | 1876541 | 1212656 | 2686608 |
| Bi | 5 | 175429 | 441198 | 837470 | 482747 | 1277382 |
| AFP-(243-261)-G2-Bi | 11 | 597 | 11221 | 98783 | 13482 | 147746 |
| | 4 | 568 | 1922 | 18589 | 2372 | 39772 |
| AFP-(243-261)-(Fuc- | 16 | 713094 | 1054362 | 1556446 | 1140986 | 1913914 |
| GlcNAc)-Bi | 6 | 437593 | 735784 | 1112834 | 796496 | 1414510 |
| AFP-(243-256)- | 19 | 614 | 17278 | 57810 | 21482 | 85366 |
| (GlcNAc-GlcNAc)-Bi | 8 | 598 | 4832 | 16958 | 6193 | 29625 |
| total AFP | 7 | 569 | 817 | 1252 | 869 | 1450 |
| AFP-L3 | 7 | 608 | 920 | 1370 | 986 | 1573 |
| AFP-L1 | 7 | 594 | 645 | 724 | 660 | 771 |

On the level of peptides, the selected antibodies can discriminate between the core-fucosylated and the non-fucolylated variants. On the level of the native antigen, very low signals were observed indicating that the epitope may not be easily accessible. Nevertheless the AFP-L3 fraction resulted in a higher signal than the AFP-L1 fraction for the antibodies 19B12 and 3C5 confirming their specificity also on the level of the natural antigen.

### Example 8: Pretreatment for Improving the Detection of AFP-L3 on Elecsys

As the native AFP-L3 antigen showed a very low signal compared to the peptide structures we speculated that the epitope region may not be readily accessible in the native AFP-L3 for the selected anti-peptide antibodies. To improve the accessibility, we have tested a variety of pretreatment conditions, which were applied to the AFP-L3 containing samples for a predefined pretreatment time with the aim to make the epitope more accessible to the antibodies.

Several pretreatment conditions were initially tested and it appeared that in particular pretreatments with protein reducing agents such as DTT or TCEP might significantly improve the signal.

To confirm whether protein reducing agents such as DTT are sufficient to facilitate the binding of the antibodies 19B12 and 3C5 and how this compares to the other antibodies tested in Example 7, the following experiment was conducted.

### Testing Protocol:

### First Step: Pretreatment

A manual sample pretreatment was conducted: 10 μl of protein sample (concentration of the protein 250 μg/mL or 25 μg/ml in the stock solution)+490 μl of pretreatment solution A or B (see below); incubation of the mixture for 30 min at room temperature.

Following biotinylated proteins have been used as samples:
1. total α-fetoprotein (AFP), containing AFP-L3 fraction, derived from a human hepatoma cell line
2. α-fetoprotein (AFP-L3) as the fucosylated variant of AFP that reacts with Lens culinaris agglutinin A
3. α-fetoprotein (AFP-L1) is the non-fucosylated variant of AFP that should not react with Lens culinaris agglutinin A

Two different pretreatment solutions have been tested with each sample:
50 mM citrate, 20 mM EDTA, 110 mM DTT pH 4.9 (Pretreatment solution A)
and as negative control: PBS+0.05% Tween 20 (Pretreatment solution B)

The end concentration of the used proteins in the sample-preachment incubate was 5000 ng/mL or 500 ng/mL (this corresponds to ca. 70 nM and 7 nM protein concentrations in the sample-pretreatment incubate). The concentration of DTT in the sample-pretreatment incubate was 107 mM.

After manual pretreatment, assay was performed on the cobas e601 analyzer using following reagents under following incubation conditions:

### Second Step: Addition of Ruthenylated AFP-L3 Antibody

40 μl of pretreated sample (from the first step)+60 μl Reagent 1+60 μl Reagent 2, incubation for 9 min

Following Reagents have been Used:
Reagent 1: mAb<AFP-L3>rRb-xx-IgG-SulfoBPRu (1 μg/mL) (Five different clones have been tested: xx: 6B6, 3A3, 3C5, 3D2, 19B12)
Reagent 2: 40 mM NaPi pH 7.5/150 mM NaCl/preserving agent/0.1% Polidocanol

Third step: Mixture of second step: +40 μl SA-Beads (streptavidin-coated microparticles). After addition of SA-Beads, the complex becomes bound to the solid phase via interaction of biotin and streptavidin, 9 min incubation.

After incubation, the reaction mixture was aspirated into the measuring cell where the microparticles are magnetically captured onto the surface of the electrode. Unbound substances are then removed. Application of a voltage to the electrode then induces electrochemiluminescent emission which is measured by a photomultiplier.

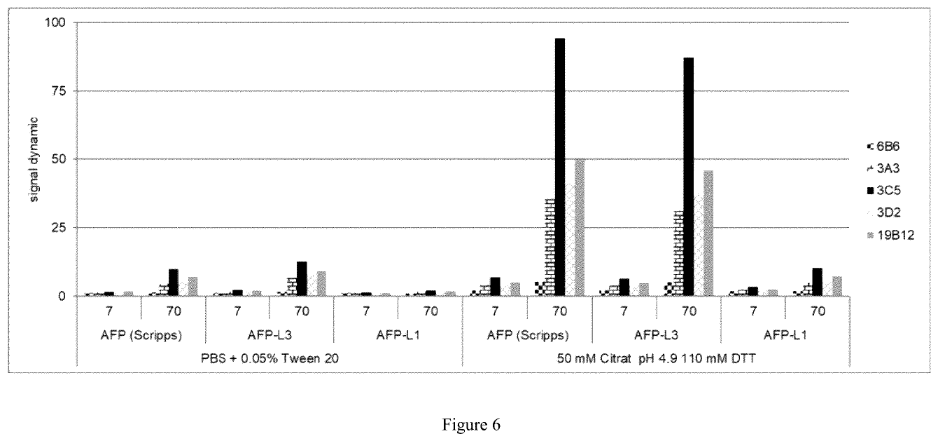

The results are depicted in FIG. 6.

### Summary of Results:

Samples pretreated with pretreatment solution containing DTT yielded much higher signals than samples pretreated with the control solution (without DTT). This indicates the improvement of the recognition of AFP-L3 by using a pretreatment solution (containing a reducing agent) for better signal detection.
The clones 3C5 and 19B12 delivered the highest signals, which confirms the better binding of these antibodies also with pretreated AFP-L3.
AFP and AFP-L3 proteins (both comprising core-fucosylated AFP) delivered much higher signals than the AFP-L1 (not core-fucosylated) protein. This confirms the specificity of the used clones as previously shown on peptide level in Biacore and Elecsys®.

### Example 9: Pretreatment Optimization

In Example 8 50 mM citrat, 20 mM EDTA, 110 mM DTT pH 4.9 were used for the pretreatment. As known in the art DTT has a much higher redox potential at basic or neutral pH but is much more stable in acidic pH (in buffer with pH<5.5 DTT is more stable). Thus, while the pretreatment solution in Example 8 shows a good stability, quite high concentrations of DTT were used.

As DTT shows high reducing activity at a pH>7, we speculated that lower DTT concentrations could be used if the pH in the sample-pretreatment incubate would be in this pH range.

To enable both reduction activity at much lower DTT concentrations and sufficient reagent stability, one pretreatment solution was divided in 2 different bottles: PT1 and PT2
PT1: 7.4 mM DTT, 2 mM EDTA pH 5.5 to have stable DTT storage conditions
PT2: Two different formulations for PT2 have been tested: 100 mM Tris pH 8.5 or 200 mM NaOH pH>13

Using this setting a pH>7.5 could be achieved in the sample-pretreatment incubate (Sample+PT1+PT2).

These pretreatment conditions were tested using a different assay format as used in Examples 7 and 8. Briefly, in step 1 the samples (here human serum without AFP-L3 or spiked with 809 ng/ml AFP-L3) were mixed with the PT1 and PT2 solution and incubated for 9 min. In step 2, R1 comprising a biotinylated AFP-specific antibody TU11 as F(ab′)₂ and R2 comprising the antibody 19B12 as a ruthenylated multivalent antibody (P8 format) were added and the mixture was incubated for 9 min. Finally, in step 3 the magnetic streptavidin microbeads were added and the resulting mixture was incubated again for 9 min.

### Used Reagents:

| | |
|---|---|
| R1: mAb<AFP>M-TU11-F(ab\grave{ })2 -Bi(linker) | 2.3 μg/ml |
| R2: mAb<AFP-L3>rRb-19B12-IgG(P8)-SulfoBPRu | 15 μg/ml |

### Testing Protocol:

30 μl sample+13 μl PT1+23 μl PT2, 9 min incubation
+51 μl R1+53 μl R2, 9 min incubation
+29 μl SA-Beads, 9 min incubation

After incubation, the reaction mixture was aspirated into the measuring cell where the microparticles are magnetically captured onto the surface of the electrode. Unbound substances are then removed. Application of a voltage to the electrode then induces electrochemiluminescent emission, which is measured by a photomultiplier.

### Results

Elecsys ® counts measurement for
Human Serum with low or high AFP-L3
protein concentration comparing 2 PT2 conditions.

| AFP-L3 | PT2: 100 mM | PT2: 200 mM |
|---|---|---|
| (ng/mL) | Tris pH 8.5 | NaOH pH > 13 |
| 0.0000 | 717 | 789 |
| 809 | 134185 | 3255 |

Both pretreatments resulted in a measurable signal but using Tris pH8.5 showed a much better performance than the use of NaOH (leading to higher pH) as PT2.

The conclusion from the experiment was that for the PT2, TRis buffer is better suitable than a more harsh alkaline solution e.g. NaOH.

Furthermore, we noted in this experiment relative to similar experiments using an IgG instead of a multivalent P8 format of the AFP-L3 antibody that the P8 format increases the signal.

### Example 10: Assessment of the Importance of the Reducing Agent DTT in the Pretreatment Solution Using 19B12 Clone

Examples 8 and 9 both used the chelator EDTA aside to DTT in the pretreatment. To get more insight in the importance of DTT for the pretreatment activity different conditions were tested using human native serum and spiked samples (master calibrator set MK1 to MK7, human serum spiked with AFP-L3 antigen increasing concentration).

The two following compositions of PT1 were tested to confirm the role of DTT for the pretreatment:
PT1 version 1: 10 mM DTT, 2 mM EDTA, pH about 4.7: with DTT
PT1 version 2: 2 mM EDTA, pH about 4.7: without DTT
PT2: 150 mM TRIS pH 8.5

### Testing Protocol:

The assay principle used was the same as in Example 9, i.e. a sandwich immunoassay.
Step 1: 30 μl sample+13 μl PT1 (version 1 or 2)+23 μl PT2 à 9 min incubation
Step 2: +51 μl R1+53 μl R2 à 9 min incubation
Step 3: +29 μl SA-Beads à 9 min incubation
R1: Biotinylated monoclonal anti-AFP antibody mAb<AFP>M-TU11-F(ab′)2-PEG24-Bi 3.4 mg/L; TRIS buffer 200 mM, pH 7.5; preservative
R2: Monoclonal anti-AFP-L3 antibody labeled with ruthenium complex mAb<AFP-L3=>rRb-19B12-IgG (P8)-SulfoBPRu 22.0 mg/L; TRIS buffer 200 mM, pH 7.5; preservative

After incubation, the reaction mixture was aspirated into the measuring cell where the microparticles are magnetically captured onto the surface of the electrode. Unbound substances are then removed. Application of a voltage to the electrode then induces electrochemiluminescent emission, which is measured by a photomultiplier.

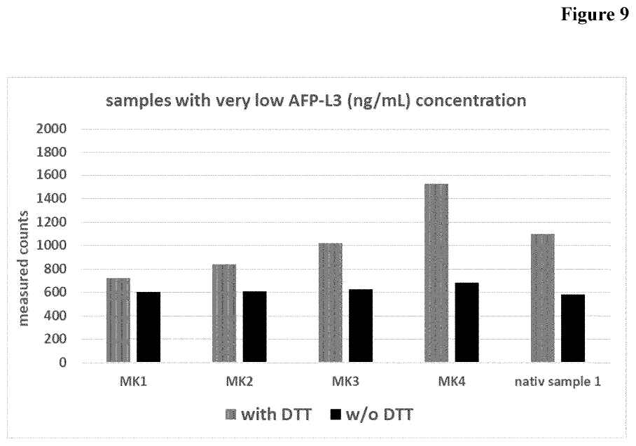

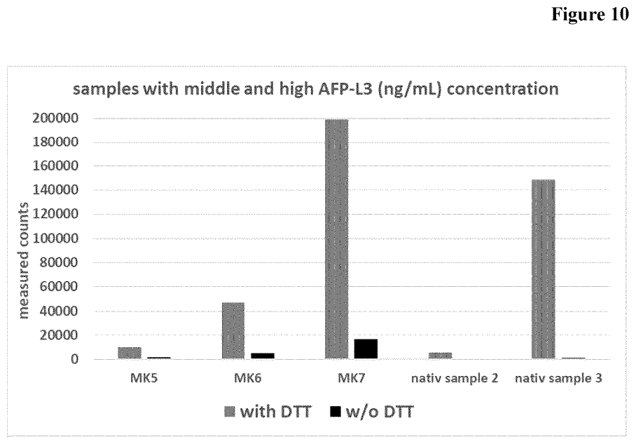

The results shown in FIGS. 9 and 10 show the crucial role of a reducing agent such as DTT for the better AFP-L3 detection both for the native and spiked samples.

### Example 11: Variation of the DTT Concentration (mM) and Tris Buffer Concentration (mM) in PT2

The goal of this experiment was evaluate different concentrations of DTT in PT1 and of Tris in PT2.

Again, a sandwich immunoassay format was used.

### Analyzed Pretreatment Conditions:

Different concentrations in PT1 were tested:
PT1: pH 5.5, DTT variation from 0 to 10 mM+EDTA 2 mM
In PT2 two different concentrations of Tris have been tested:
PT2: pH 9.0, Tris Buffer (50 or 200 mM)
For reagents 1 and 2 constant conditions have been used:
Reagent 1: mAb<AFP>M-TU11-F(ab′)2-Bi(PEG24) (2.5 μg/mL)
Reagent 2: mAb<AFP-L3>rRb-19B12-IgG(P8)-SulfoBPRu (15 μg/mL)

### Measured Sample:

Human Serum (=“HS”) spiked with AFP-L3 Protein, pH set to 8

### Testing Protocol:

First incubation: 39 μl sample+16 μl PT1+16 μl PT2, 9 min incubation
Second incubation: Mixture 1^{st} incubation+50 μl R1+52 μl R2, 9 min incubation
Third incubation: Mixture second incubation+30 μl SA-Beads, 9 min incubation

After incubation, the reaction mixture was aspirated into the measuring cell where the microparticles are magnetically captured onto the surface of the electrode. Unbound substances are then removed. Application of a voltage to the electrode then induces electrochemiluminescent emission, which is measured by a photomultiplier.

The tested DTT concentrations are summarized in Table 8, below.

TABLE 8
DTT concentration variation.

| | | | | |
|---|---|---|---|---|
| DTT Conc. mM in PT1 flask (before | 0 | 3 | 6 | 10 |
| incubation with the sample) | | | | |
| End Conc. DTT in Sample- Pretreatment - | 0 | 0.676 | 1.352 | 2.253 |
| incubate: (during the first incubation) | | | | |
| End Conc. DTT in total reagent - incubate: | 0 | 0.237 | 0.473 | 0.788 |
| (after third incubation, end concentration | | | | |
| in the measurement) | | | | |

### Results:

Measured signals for different tested conditions are summarized in table 9:

Elecsys ® counts measurement for Human Serum (=HS) (different pH
adjusted) with high AFP-L3 protein concentration comparing different
conditions [DTT concentration in PT1 and Tris concentration in PT2]

| PT1 | | | HS pH 8.0 | |
|---|---|---|---|---|
| EDTA | pH 5.5 | PT2 pH 9 | 0 ng/mL | |
| [mM] | DTT [mM] | Tris [mM] | AFP-L3 | HS pH 8.0 |
| 2 | 0 | 50 | 750 | 7929 |
| | | 200 | 761 | 7684 |
| | 3 | 50 | 741 | 120884 |
| | | 200 | 728 | 125011 |
| | 6 | 50 | 713 | 96751 |
| | | 200 | 717 | 60056 |
| | 10 | 50 | 715 | 11765 |
| | | 200 | 697 | 32065 |

### Conclusion:

As shown in Table 9 different DTT concentrations can be used for the pretreatment. Concentrations as low as 0.676 mM in the pretreatment-sample mixtures were sufficient to increase the signal vs. no DTT. This indicates that even lower concentrations of DTT can be used.

In the present Example DTT concentrations from 1 to 10 mM in PT1 have been tested. In the previous Examples, in particular in Example 8 a higher DTT concentration of 110 mM in the stock has been used. This illustrates that a broad range of DTT concentrations can be used for improving the signal, even though to different extents dependent on the concentration and also the pH of the pretreatment-sample incubate.

EDTA was found to stabilize DTT in PT1, i.e. 2 mM were used.

### Example 12: Method Comparison of Prototype Elecsys® AFP-L3 Assays to Wako Instrument

A clinical panel (1000 clinical samples containing HCC cases and controls) was measured with two different prototype Elecsys® AFP-L3 assays and for comparison with the μTASWako AFP-L3 (Fujifilm Wako Pure Chemical Corporation) The prototype assays were identical with the only exception that either 19B12 or 3C5 P8-format multivalent antibodies were employed.

The assay set up was as follows:

TABLE 10
Assay protocol

| Step 1 | Step 2 | Step 3 |
|---|---|---|
| 16 μl PT1 + 25 μl | Step 1 + 50 μl | Step 2 + 30 μl |
| PT2 + 30 μl sample | R1 + 52 μl R2 | beads |

### Used Reagents:

PT1: 6 mM DTT, 2 mM EDTA, pH 5.5
PT2: 100 mM TRIS pH 8.5

Two assay prototypes were evaluated as shown in Table 11.

TABLE 11
prototype assays a and b

| | Prototype a | Prototype b |
|---|---|---|
| R1 | mAb<AFP>M-TU11-F(ab′)2- | mAb<AFP>M-TU11-F(ab′)2- |
| buffer | PEG24-Bi, | PEG24-Bi, |
| (pH 7.2): | 2.5 μg/mL | 1.75 mg/mL |
| R2 | mAb<AFP-L3>rRb-19B12- | mAb<AFP-L3>rRb-3C5-IgG |
| buffer | IgG (P8)-SulfoBPRu | (P8)-SulfoBPRu |
| (pH 7.2) | 15 μg/mL | 15 μg/mL |

The samples were additionally measured by μTASWako AFP-L3 (Fujifilm Wako Pure Chemical Corporation) according to the manufacturer's instruction to determine the content of AFP-L3 in % and total concentration of AFP in ng/mL. From the obtained μTASWako values for % AFP-L3 and total AFP concentration in ng/mL, the AFP-L3 concentration for Wako in ng/mL was calculated. Elecsys® AFP-L3 values were obtained directly in ng/mL, using both prototypes a and b as described in example 11.

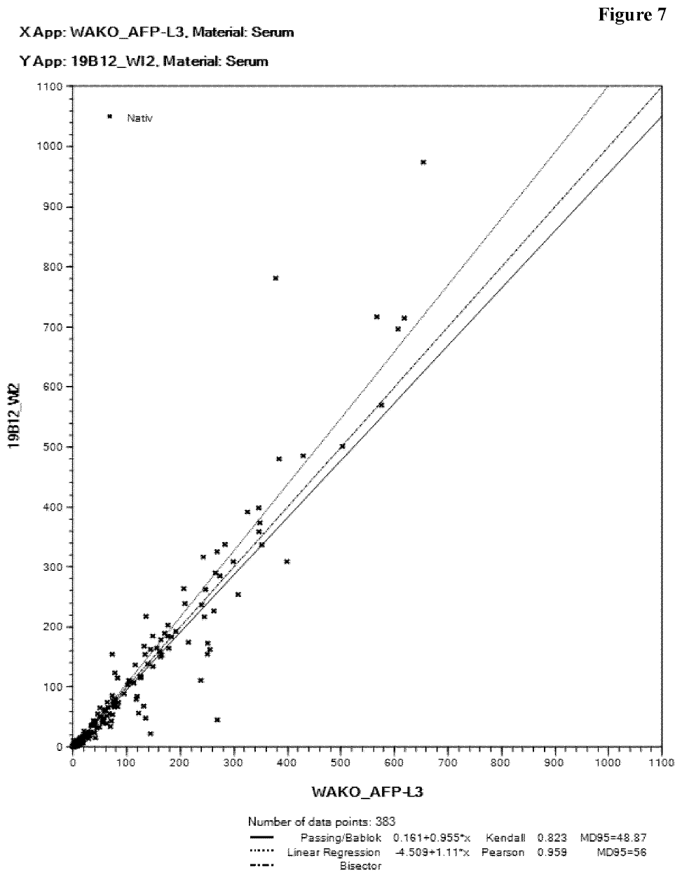

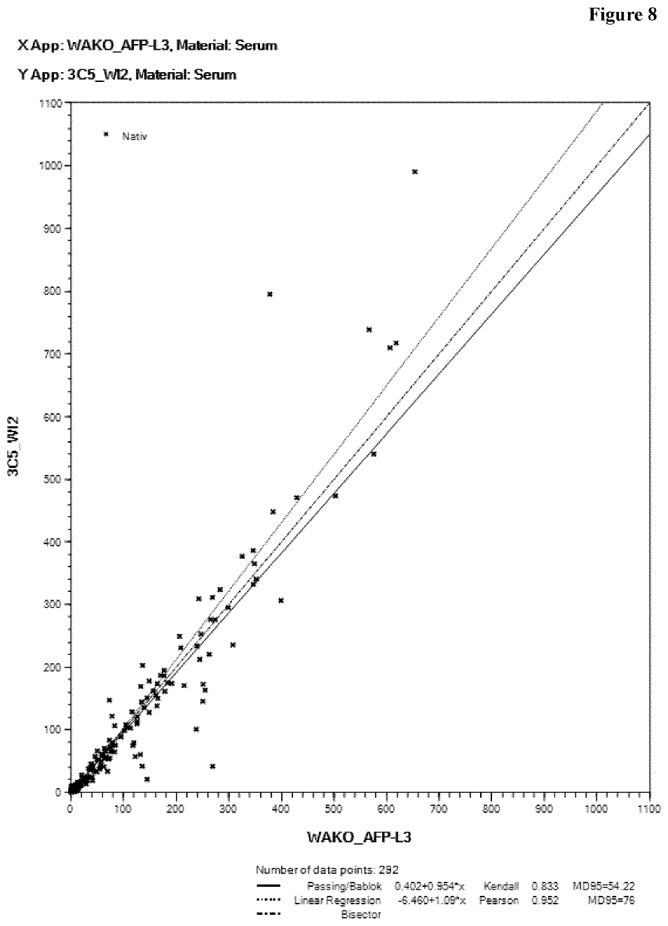

Both assay prototypes have shown very similar results and showed a very good correlation with the μTASWako AFP-L3 (Fujifilm Wako Pure Chemical Corporation), see FIGS. 7 and 8.

The results of this method comparison confirm that the prototypes show a good correlation with the launched and clinically validated μTASWako AFP-L3 (Fujifilm Wako Pure Chemical Corporation). This confirms also that essentially AFP-L3 is detected by the assays of the invention even though no LCA lectin is used.

### Example 13: Performance of Assay in a Clinical Panel

To further confirm the clinical value of the Elecsys® AFP-L3 prototype assays using the antibodies 19B12 and 3C5 a clinical panel was analyzed and the diagnostic performance for detecting early HCC was assessed. This panel included 96 patients with liver disease, of which 38 had HCC and 58 had liver disease but no diagnosis of HCC (control).

Exemplary a 19B12 based prototype was used.

### Testing Protocol:

sandwich immunoassay
Step 1: 30 μl sample+13 μl PT1+23 μl PT2 à 9 min incubation
Step 2: +51 μl R1+53 μl R2 à 9 min incubation
Step 3: +29 μl SA-Beads à 9 min incubation

### Reagents

PT1: 10 mM DTT, 2 mM EDTA, pH<5.5
PT2: 150 mM TRIS pH 8.5
R1: Biotinylated monoclonal anti-AFP antibody TU11; TRIS buffer 200 mM, pH 7.5; preservative
R2: Monoclonal anti-AFP-L3 antibody 19B12 labeled with ruthenium complex as multivalent P8 antibody; TRIS buffer 200 mM, pH 7.5; preservative

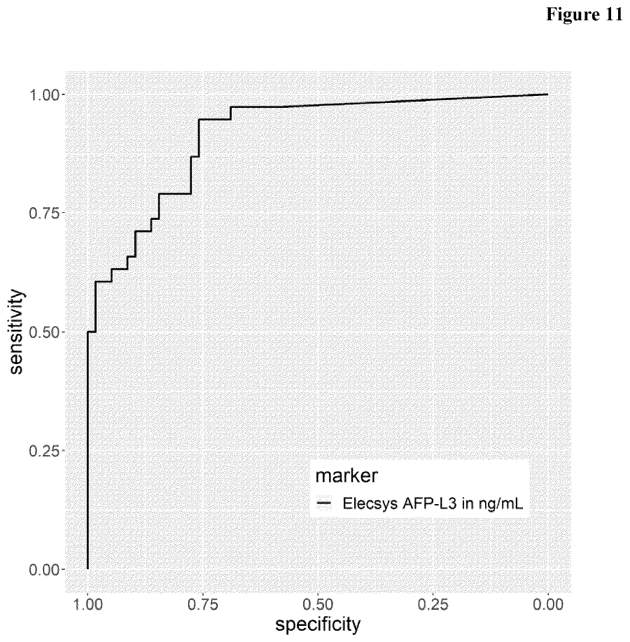

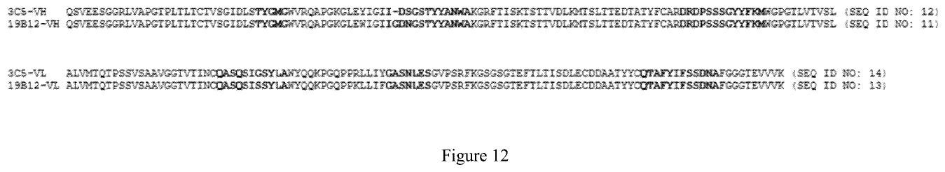

The ROC curve is shown in FIG. 11. The AUC (are under the curve) for the AFP-L3 assay based on 19B12 was 91.4% (CI: 85.8-97), which proves very good performance for detecting HCC of the assay in this panel.

TABLE 12
Clinical performance (sensitivity and specificity)
of Elecsys ® AFP-L3 assay at predefined
specificity criteria as measured in panel A

| Criteria | Cut-off | Sensitivity | Specificity |
|---|---|---|---|
| 95% Spec | 5.27 ng/ml | 63.2 | 94.8 |
| 90% Spec | 3.28 ng/ml | 71.1 | 89.7 |

1. A monoclonal antibody or antigen binding fragment thereof specifically binding to α-1,6-core-fucosylated alpha-fetoprotein (AFP) or a partial sequence of AFP comprising said α-1,6-core-fucosylation, wherein the monoclonal antibody or antigen binding fragment comprises
(i) a heavy chain variable domain (VH) comprising a CDR-H1 having the amino acid sequence of SEQ ID NO: 3; a CDR-H2 having the amino acid sequence of SEQ ID NO: 4 or 5; and a CDR-H3 having the amino acid sequence of SEQ ID NO: 6; and
(ii) a light chain variable domain (VL) comprising a CDR-L1 having the amino acid sequence of SEQ ID NO: 7 or 8; a CDR-L2 having the amino acid sequence of SEQ ID NO: 9; and a CDR-L3 having the amino acid sequence of SEQ ID NO: 10.

2. The monoclonal antibody or antigen binding fragment according to claim 1, wherein the partial sequence of AFP comprising said α-1,6-core-fucosylation comprises or consists of a glycopeptide of Formula I,

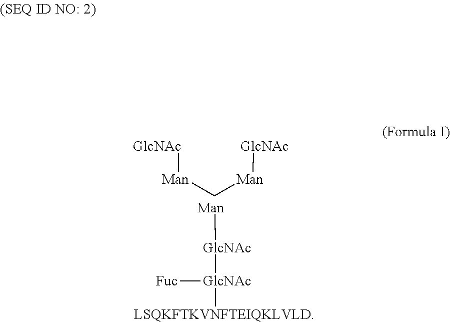

3. The monoclonal antibody or antigen binding fragment according to claim 2, wherein the monoclonal antibody or antigen binding fragment specifically bind the glycopeptide of Formula I and the glycopeptide of Formula II,

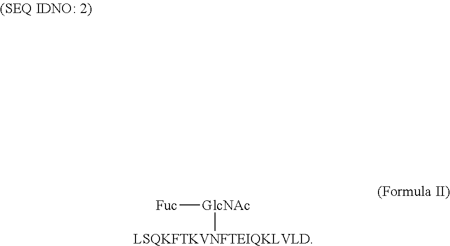

4. The monoclonal antibody or antigen binding fragment according to claim 3, wherein the ratio between the K_{D} for the binding to glycopeptide of Formula II and the glycopeptide of Formula I is at least 2.

5. The monoclonal antibody or antigen binding fragment according to claim 1, wherein
(i) the heavy chain variable domain (VH) has an amino acid sequence with at least 80%, at least 85%, at least 90%, or at least 95% sequence identity to SEQ ID NO:11 or 12; and
(ii) the light chain variable domain (VL) has an amino acid sequence with at least 80%, at least 85%, at least 90%, or at least 95% sequence identity to SEQ ID NO: 13 or 14.

6. The monoclonal antibody or antigen binding fragment according claim 1, wherein
(i) the heavy chain variable domain (VH) has the amino acid sequence SEQ ID NO: 11 or 12; and
(ii) the light chain variable domain (VL) has the amino acid sequence of SEQ ID NO: 13 or 14.

7. The monoclonal antibody or antigen binding fragment according to claim 1, which binds to the glycopeptide of Formula I, 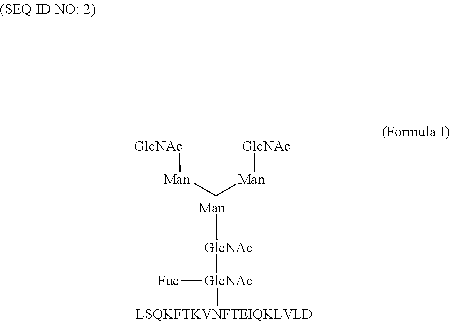
with a K_{D} of 100 nM or less, 20 nM or less, 10 nM or less or 3.1 nM or less, optionally wherein the K_{D} is measured at 37° C.

8. The monoclonal antibody or antigen binding fragment according to claim 1, which binds to the glycopeptide of Formula I, 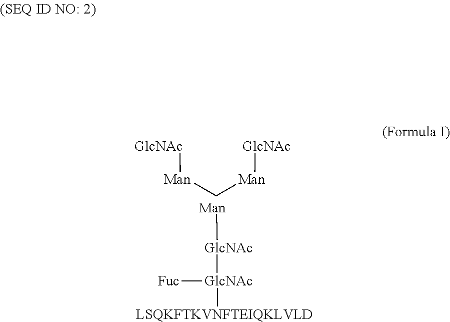
with a K_{D} that is less than 10, 8, 6, 4, or 2 times the K_{D} of a rabbit IgG antibody comprising the heavy chain variable domain of SEQ ID NO: 11 or 12 and a light chain variable domain of SEQ ID NO: 13 or 14 towards the glycopeptide of Formula I, wherein the K_{D} values are measured under the same conditions and using the same method.

9. A polynucleotide or a set of polynucleotides encoding
(i) the heavy chain or heavy chain variable domain of the monoclonal antibody or antigen binding fragment according to claim 1; and/or
(ii) the light chain or light chain variable domain of the monoclonal antibody or antigen binding fragment according to claim 1.

10. A vector comprising the polynucleotide or the set of polynucleotides according to claim 9.

11. A host cell comprising a polynucleotide or a set of polynucleotides encoding (i) the heavy chain or heavy chain variable domain of the monoclonal antibody or antigen binding fragment according to claim 1; and/or (ii) the light chain or light chain variable domain of the monoclonal antibody or antigen binding fragment according to claim 1, and expressing the antibody or antigen binding fragment of claim 1.

12. A method of producing the monoclonal antibody or antigen binding fragment according to claim 1 comprising culturing a host cell comprising a polynucleotide or a set of polynucleotides encoding (i) the heavy chain or heavy chain variable domain of the monoclonal antibody or antigen binding fragment according to claim 1; and/or (ii) the light chain or light chain variable domain of the monoclonal antibody or antigen binding fragment according to claim 1, and isolating said antibody or antigen binding fragment.

13. A composition comprising the antibody or antigen binding fragment according to claim 1.

14. (canceled)

15. An in vitro immunoassay method for detecting α-1,6-core-fucosylated AFP or a partial AFP sequence comprising said α-1,6-core-fucosylation in a sample using the antibody or antigen binding fragment as defined in claim 1.

16. A pretreatment reagent for treating a sample comprising α-1,6-core-fucosylated AFP, said pretreatment reagent comprising a reducing agent.

17. A kit comprising the antibody or antigen binding fragment as defined in claim 1.

18. The monoclonal antibody or antigen binding fragment according to claim 5, wherein
(i) the heavy chain variable domain (VH) has an amino acid sequence with at least 97.5% sequence identity to SEQ ID NO: 11 or 12; and
(ii) the light chain variable domain (VL) has an amino acid sequence with at least 97.3% sequence identity to SEQ ID NO: 13 or 14.

19. The in vitro immunoassay method for detecting α-1,6-core-fucosylated AFP or a partial AFP sequence of claim 15, wherein the sample is pretreated with a pretreatment reagent comprising a reducing agent.

20. The in vitro immunoassay method for detecting α-1,6-core-fucosylated AFP or a partial AFP sequence of claim 19, wherein the reducing agent is a protein reducing agent.

21. The pretreatment reagent for treating a sample comprising α-1,6-core-fucosylated AFP according to claim 16, wherein the reducing agent is a protein reducing agent.


SEQUENCE LISTING (27 sequences)

SEQ ID NO: 1 - protein, 609 aa, Homo sapiens
MKWVESIFLIFLLNFTESRTLHRNEYGIASILDSYQCTAEISLADLATIFFAQFVQEATYKEVSKMVKDALTAIEKPTGDEQSSGCLENQLPAFLEELCHEKEILEKYGHSDCCSQSEEGRHNCFLAHKKPTPASIPLFQVPEPVTSCEAYEEDRETFMNKFIYEIARRHPFLYAPTILLWAARYDKIIPSCCKAENAVECFQTKAATVTKELRESSLLNQHACAVMKNFGTRTFQAITVTKLSQKFTKVNFTEIQKLVLDVAHVHEHCCRGDVLDCLQDGEKIMSYICSQQDTLSNKITECCKLTTLERGQCIIHAENDEKPEGLSPNLNRFLGDRDFNQFSSGEKNIFLASFVHEYSRRHPQLAVSVILRVAKGYQELLEKCFQTENPLECQDKGEEELQKYIQESQALAKRSCGLFQKLGEYYLQNAFLVAYTKKAPQLTSSELMAITRKMAATAATCCQLSEDKLLACGEGAADIIIGHLCIRHEMTPVNPGVGQCCTSSYANRRPCFSSLVVDETYVPPAFSDDKFIFHKDLCQAQGVALQTMKQEFLINLVKQKPQITEEQLEAVIADFSGLLEKCCQGQEQEVCFAEEGQKLISKTRAALGV

SEQ ID NO: 2 - protein, 19 aa, Homo sapiens
LSQKFTKVNFTEIQKLVLD

SEQ ID NO: 3 - protein, 5 aa, synthetic construct
  REGION 1..5: note = CDR-H1 19B12 and 3C5
TYGMG

SEQ ID NO: 4 - protein, 14 aa, synthetic construct
  REGION 1..14: note = CDR-H2 19B12
IIGDNGSTYYANWA

SEQ ID NO: 5 - protein, 13 aa, synthetic construct
  REGION 1..13: note = CDR-H2 3C5
IIDSGSTYYANWA

SEQ ID NO: 6 - protein, 13 aa, synthetic construct
  REGION 1..13: note = DRDPSSSGYYFKM
DRDPSSSGYYFKM

SEQ ID NO: 7 - protein, 11 aa, synthetic construct
  REGION 1..11: note = CDR-L1 19B12
QASQSISSYLA

SEQ ID NO: 8 - protein, 11 aa, synthetic construct
  REGION 1..11: note = CDR-L1 3C5
QASQSIGSYLA

SEQ ID NO: 9 - protein, 7 aa, synthetic construct
  REGION 1..7: note = CDR-L2 19B12 and 3C5
GASNLES

SEQ ID NO: 10 - protein, 12 aa, synthetic construct
  REGION 1..12: note = CDR-L3 19B12 and 3C5
QTAFYIFSSDNA

SEQ ID NO: 11 - protein, 118 aa, synthetic construct
  REGION 1..118: note = VH sequence 19B12
QSVEESGGRLVAPGTPLTLTCTVSGIDLSTYGMGWVRQAPGKGLEWIGIIGDNGSTYYANWAKGRFTISKTSTTVDLKMTSLTTEDTATYFCARDRDPSSSGYYFKMWGPGTLVTVSL

SEQ ID NO: 12 - protein, 117 aa, synthetic construct
  REGION 1..117: note = VH sequence 3C5
QSVEESGGRLVAPGTPLTLTCTVSGIDLSTYGMGWVRQAPGKGLEYIGIIDSGSTYYANWAKGRFTISKTSTTVDLKMTSLTTEDTATYFCARDRDPSSSGYYFKMWGPGTLVTVSL

SEQ ID NO: 13 - protein, 110 aa, synthetic construct
  REGION 1..110: note = VL sequence 19B12
ALVMTQTPSSVSAAVGGTVTINCQASQSISSYLAWYQQKPGQPPKLLIFGASNLESGVPSRFKGSGSGTEFTLTISDLECDDAATYYCQTAFYIFSSDNAFGGGTEVVVK

SEQ ID NO: 14 - protein, 110 aa, synthetic construct
  REGION 1..110: note = VL sequence 3C5
ALVMTQTPSSVSAAVGGTVTINCQASQSIGSYLAWYQQKPGQPPRLLIYGASNLESGVPSRFKGSGSGTEFTLTISDLECDDAATYYCQTAFYIFSSDNAFGGGTEVVVK

SEQ ID NO: 15 - protein, 29 aa, synthetic construct
  REGION 1..29: note = FW1 of VH of 19B12 and 3C5
QSVEESGGRLVAPGTPLTLTCTVSGIDLS

SEQ ID NO: 16 - protein, 14 aa, synthetic construct
  REGION 1..14: note = FW2 of VH of 19B12
WVRQAPGKGLEWIG

SEQ ID NO: 17 - protein, 14 aa, synthetic construct
  REGION 1..14: note = FW2 of VH of 3C5
WVRQAPGKGLEYIG

SEQ ID NO: 18 - protein, 32 aa, synthetic construct
  REGION 1..32: note = FW3 of VH of 19B12 and 3C5
KGRFTISKTSTTVDLKMTSLTTEDTATYFCAR

SEQ ID NO: 19 - protein, 11 aa, synthetic construct
  REGION 1..11: note = FW4 of VH of 19B12 and 3C5
WGPGTLVTVSL

SEQ ID NO: 20 - protein, 23 aa, synthetic construct
  REGION 1..23: note = FW1 of VL of 19B12 and 3C5
ALVMTQTPSSVSAAVGGTVTINC

SEQ ID NO: 21 - protein, 15 aa, synthetic construct
  REGION 1..15: note = FW2 of VL of 19B12
WYQQKPGQPPKLLIF

SEQ ID NO: 22 - protein, 15 aa, synthetic construct
  REGION 1..15: note = FW2 of VL of 3C5
WYQQKPGQPPRLLIY

SEQ ID NO: 23 - protein, 32 aa, synthetic construct
  REGION 1..32: note = FW3 of VL of 19B12 and 3C5
GVPSRFKGSGSGTEFTLTISDLECDDAATYYC

SEQ ID NO: 24 - protein, 10 aa, synthetic construct
  REGION 1..10: note = FW4 of VL of 19B12 and 3C5
FGGGTEVVVK

SEQ ID NO: 25 - protein, 9 aa, Homo sapiens
TKVNFTEIQ

SEQ ID NO: 26 - protein, 14 aa, Homo sapiens
LSQKFTKVNFTEIQ

SEQ ID NO: 27 - protein, 16 aa, synthetic construct
  REGION 1..16: note = Linker
GGGSGGGSGGGSGGGS